(19) United States
(12) Patent Application Publication
(10) Pub. No.: US 2020/0339691 A1
(43) Pub. Date: Oct. 29, 2020

(54) PROTEINACEOUS MOLECULES AND USES THEREFOR

(71) Applicant: EPIAXIS THERAPEUTICS PTY LTD, Herston, Queensland (AU)

(72) Inventors: Sudha RAO, Red Hill, Queensland (AU); Peter MILBURN, O' Connor, Australian Capital Territory (AU)

(21) Appl. No.: 16/962,167

(22) PCT Filed: Jan. 15, 2019
(86) PCT No.: PCT/AU2019/050024

§ 371 Date: Jul. 14, 2020

(30) Foreign Application Priority Data
Jan. 15, 2018 (AU) 2018900108

(51) Int. Cl. C07K 16/28

(52) U.S. Cl. CPC C07K 16/2818; C07K 2317/76; C07K 16/2827

(57) ABSTRACT

Disclosed are proteinaceous molecules corresponding to an acetylation site and their use for inhibiting or reducing the nuclear localization of a nuclear localizable polypeptide, such as PD-1, PD-L1 and PD-L2. This invention also relates to the use of the proteinaceous molecules for altering at least one of (i) formation; (ii) proliferation; (iii) maintenance; (iv) epithelial to mesenchymal cell transition (EMT); or (v) mesenchymal to epithelial cell transition (MET) of a PD-1-, PD-L1- or PD-L2-overexpressing cell, and for treating or preventing a cancer in a subject.

This application claims priority to Australian Provisional Application No. 2018900108 entitled “Proteinaceous Molecules and Uses Therefor” filed 15 Jan. 2018, the contents of which are incorporated herein by reference in their entirety.

### FIELD OF THE INVENTION

This invention relates generally to proteinaceous molecules corresponding to an acetylation site and their use for inhibiting or reducing the nuclear localization of a nuclear localizable polypeptide, such as PD-1, PD-L1 and PD-L2. This invention also relates to the use of the proteinaceous molecules for altering at least one of (i) formation; (ii) proliferation; (iii) maintenance; (iv) epithelial to mesenchymal cell transition (EMT); or (v) mesenchymal to epithelial cell transition (MET) of a PD-1-, PD-L1- or PD-L2-overexpressing cell, and for treating or preventing a cancer in a subject.

### BACKGROUND OF THE INVENTION

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Programmed cell death protein-1 (PD-1) plays an important role in regulation of the immune system through its ability to regulate T cell activation and reduce the immune response. PD-1 is expressed on activated T cells (including immunosuppressive CD4+ T cells (Treg) and exhausted CD8+ T cells), B cells, myeloid dendritic cells (MDCs), monocytes, thymocytes and natural killer (NK) cells (Gianchecchi et al. (2013) Autoimmun. Rev., 12: 1091-1100).

The PD-1 signaling pathway contributes to the maintenance of central and peripheral tolerance in normal individuals, thereby avoiding destruction of normal host tissue. In the thymus, the interaction of PD-1 and its ligands suppresses positive selection, thereby inhibiting the transformation of CD4− CD8− double negative cells to CD4+ CD8+ double positive T cells (Keir et al. (2005) J. Immunol 175: 7329-7379). Inhibition of self-reactive and inflammatory effector T cells that escape negative selection to avoid collateral immune-mediated tissue damage is dependent on the PD-1 signaling pathway (Keir et al. (2006) Exp. Med., 203: 883-895).

PD-1 is bound by two ligands: programmed cell death ligand-1 (PD-L1; B7-H1; CD274) and programmed cell death ligand-2 (PD-L2; B7-DC; CD273). PD-L1 is expressed on various cell types, including T cells, B cells, dendritic cells, macrophages, epithelial cells and endothelial cells (Chen et al. (2012) Cliin Cancer Res, 18(24): 6580-6587; Herzberg et al. (2016) The Oncologist, 21: 1-8). PD-L1 expression is also upregulated in many types of tumor cells and other cells in the local tumor environment (Herzberg et al. (2016) The Oncologist, 21: 1-8). PD-L2 is predominantly expressed on antigen-presenting cells such as monocytes, macrophages and dendritic cells, but expression may also be induced on a wide variety of other immune cells and non-immune cells depending on microenvironmental stimuli (Herzberg et al. (2016) The Oncologist, 21: 1-8; Kinter et al. (2008) J. Immunol., 181: 6738-6746; Zhong et al. (2007) Eur. J. Immunol., 37: 2405-2410; Messal et al. (2011) Mol. Immunol., 48: 2214-2219; Lesterhuis et al. (2011) Mol. Immunol., 49: 1-3).

PD-1, PD-L1 and PD-L2 are overexpressed by malignant cells and other cells in the local tumor environment. PD-1 is highly expressed on a large proportion of tumor-infiltrating lymphocytes (TILs) from many different tumor types and suppresses local effector immune responses. TIL expression of PD-1 is associated with impaired effector function (cytokine production and cytotoxic efficacy against tumor cells) and/or poor outcome in numerous tumor types (Thompson et al. (2007) Clin Cancer Res, 13(6): 1757-1761; Shi et al. (2011) Int. J. Cancer, 128: 887-896). PD-L1 expression has been found to strongly correlate with poor outcome in many tumor types, including kidney, ovarian, bladder, breast, urothelial, gastric and pancreatic cancer (Keir et al. (2008) Annu. Rev. Immunol., 26: 677-704; Shi et al. (2011) Int. J. Cancer, 128: 887-896). PD-L2 has been shown to be upregulated in a subset of tumors and has also been linked to poor outcome.

Interestingly, nuclear PD-L1 expression has been shown to be associated with short survival duration and chemoresistance in several tumor types, including prostate, colorectal and breast cancer (Satelli, et al. (2016) Scientific Reports, 6: 28910; Ghebeh, et al. (2010) Breast Cancer Res., 12: R48).

Accordingly, members of the PD-1 signaling pathway are important therapeutic targets for the treatment of cancer and new therapeutic agents targeting this pathway, particularly the nuclear localization of the members of the PD-1 signaling pathway, are desired.

### SUMMARY OF THE INVENTION

The present invention is predicated in part on the discovery that proteinaceous molecules comprising an amino acid sequence corresponding to an acetylation site of PD-L1 inhibit or reduce the nuclear localization of PD-L1, PD-L2 and PD-1. Accordingly, the inventors have conceived that a proteinaceous molecule comprising an amino acid sequence corresponding to an acetylation site can be used to inhibit nuclear localization of a nuclear localizable polypeptide, wherein acetylation of an acetylation site of the nuclear localizable polypeptide increases its nuclear localization in a cell. The inventors have also conceived that the proteinaceous molecules can be used for the treatment of a cancer in a subject.

Accordingly, in one aspect of the invention, there is provided a method of inhibiting or reducing nuclear localization of a nuclear localizable polypeptide wherein acetylation of an acetylation site of the nuclear localizable polypeptide increases its nuclear localization in a cell, comprising contacting the cell with a proteinaceous molecule comprising, consisting or consisting essentially of an amino acid sequence corresponding to an acetylation site.

In yet another aspect, there is provided a method of inhibiting or reducing the nuclear localization of PD-1, PD-L1 or PD-L2 in a PD-1-, PD-L1- or PD-L2-overexpressing cell, comprising contacting the cell with a proteinaceous molecule comprising, consisting or consisting essentially of an amino acid sequence corresponding to an acetylation site.

In still another aspect, there is provided a method of altering at least one of (i) formation; (ii) proliferation; (iii) maintenance; (iv) EMT; (v) MET; or (vi) viability of a PD-1-, PD-L1- or PD-L2-overexpressing cell, comprising contacting said cell with a formation-, proliferation-, maintenance-, EMT-, MET-, or viability-modulating amount of a proteinaceous molecule comprising, consisting or consisting essentially of an amino acid sequence corresponding to an acetylation site.

In a further aspect, there is provided a method of treating or preventing a cancer in a subject wherein the cancer comprises at least one PD-1-, PD-L1- or PD-L2-overexpressing cell, comprising administering to the subject a proteinaceous molecule comprising, consisting or consisting essentially of an amino acid sequence corresponding to an acetylation site.

In a still further aspect the invention provides a method of producing a proteinaceous molecule that inhibits or reduces nuclear localization of a nuclear localizable polypeptide wherein acetylation of an acetylation site of the nuclear localizable polypeptide increases its nuclear localization in a cell, the method comprising:
a) contacting a cell with a proteinaceous molecule comprising, consisting or consisting essentially of an amino acid sequence corresponding to an acetylation site; and
b) detecting a reduction in or inhibition of the nuclear localization of the nuclear localizable polypeptide in the cell relative to a normal or reference level of nuclear localization in the absence of the proteinaceous molecule.

In yet another aspect, there is provided a method of producing a proteinaceous molecule that inhibits or reduces nuclear localization of a nuclear localizable polypeptide wherein acetylation of an acetylation site of the nuclear localizable polypeptide increases its nuclear localization in a cell, the method comprising:
a) contacting a cell with a proteinaceous molecule comprising, consisting or consisting essentially of an amino acid sequence corresponding to residues 255 to 271 of PD-L1; and
b) detecting a reduction in or inhibition of the nuclear localization of the nuclear localizable polypeptide in the cell relative to a normal or reference level of nuclear localization in the absence of the proteinaceous molecule.

In still another aspect, the invention provides a method of producing a proteinaceous molecule that inhibits or reduces at least one of formation, proliferation, viability or EMT of a cancer stem cell, the method comprising:
a) contacting a cancer stem cell with a proteinaceous molecule comprising, consisting or consisting essentially of an amino acid sequence corresponding to residues 255 to 271 of PD-L1; and
b) detecting a reduction in or inhibition of the formation, proliferation or EMT of the cancer stem cell relative to a normal or reference level of formation, proliferation, viability or EMT of the cell in the absence of the proteinaceous molecule.

The invention also contemplates an isolated or purified proteinaceous molecule represented by Formula I:

Z₁X₁X₂X₃X₄FX₅X₆X₇X₈X₉X₁₀X₁₁X₁₂X₁₃X₁₄X₁₅X₁₆Z₂ (I)

wherein:
Z₁ and Z₂ are independently absent or are independently selected from at least one of a proteinaceous moiety comprising from about 1 to about 50 amino acid residues (and all integer residues in between), and a protecting moiety;
X₁ is absent or is selected from small amino acid residues including A, G, S, T and modified forms thereof, and hydrophobic amino acid residues including M, Nle, I, L, V, F, Y, W and modified forms thereof;
X₂ is selected from small amino acid residues including A, G, S, T and modified forms thereof, and charged amino acid residues including K, R, D, E and modified forms thereof;
X₃ is selected from any amino acid residue;
X₄ is selected from charged amino acid residues including K, R, D, E and modified forms thereof, and hydrophobic amino acid residues including M, Nle, I, L, V, F, Y, W and modified forms thereof;
X₅ is selected from any amino acid residue;
X₆ is selected from charged amino acid residues including K, R, D, E and modified forms thereof, and hydrophobic amino acid residues including M, Nle, I, L, V, F, Y, W and modified forms thereof;
X₇ is selected from charged amino acid residues including K, R, D, E and modified forms thereof, and hydrophobic amino acid residues including M, Nle, I, L, V, F, Y, W and modified forms thereof;
X₈ is selected from small amino acid residues including A, G, S, T and modified forms thereof, basic amino acid residues including K, R, Orn and modified forms thereof, and amino acid residues with an amide-containing side chain including N, Q, Orn(Ac), K(Ac) and modified forms thereof;
X₉ is selected from small amino acid residues including G, S, T and modified forms thereof, charged amino acid residues including K, R, D, E and modified forms thereof, and hydrophobic amino acid residues including M, Nle, I, L, V, F, Y, W and modified forms thereof;
X₁₀ is selected from any amino acid residue;
X₁₁ is selected from any amino acid residue;
X₁₂ is selected from charged amino acid residues including K, R, D, E and modified forms thereof, and hydrophobic amino acid residues including M, Nle, I, L, V, F, Y, W and modified forms thereof;
X₁₃ is selected from any amino acid residue;
X₁₄ is selected from any amino acid residue;
X₁₅ is selected from charged amino acid residues including K, R, D, E and modified forms thereof, and hydrophobic amino acid residues including M, Nle, I, L, V, F, Y, W and modified forms thereof; and
X₁₆ is selected from basic amino acid residues including K, R and modified forms thereof.

In some embodiments, the proteinaceous molecule has any one or more activities selected from the group consisting of: (i) increasing cell death; (ii) increasing MET; (iii) reducing or inhibiting EMT; (iv) inhibiting or reducing maintenance; (v) inhibiting or reducing proliferation; (vi) increasing differentiation; (vii) inhibiting or reducing formation; or (viii) reducing viability of a PD-1-, PD-L1- or PD-L2-overexpressing cell.

### BRIEF DESCRIPTION OF THE DRAWINGS

FIG. 1 is a photographic (FIG. 1A) and graphical (FIGS. 1B to 1D) representation of localization of PD-L1 and cell surface vimentin (CSV) in metastasis initiating cells (MICs) isolated from liquid biopsies of metastatic breast cancer patients. Samples were taken from patients at intervals of 1 week, 3 weeks and 6 weeks for each of the 10 patients (P1-P10). FIG. 1A is a photographic representation of the localization of PD-L1 and CSV in patients P1-P10 at 6 weeks, FIG. 1B depicts the total nuclear fluorescence (TNFI) of PD-L1 for samples taken at 1, 3 and 6 weeks, FIG. 1C depicts the ratio of nuclear to cytoplasmic fluorescence (Fn/c) of PD-L1 for samples taken at 1, 3 and 6 weeks, and FIG. 1D depicts the total cytoplasmic fluorescence (TCFI) of CSV for samples taken at 1, 3 and 6 weeks (n≥5-10 individual cells per sample). Data is shown as mean±SE grouped into the time point of collection. Representative images for each condition are shown.

FIG. 2 is a photographic (FIG. 2A) and graphical (FIG. 2B) representation of localization of PD-L1 and CSV in MICs isolated from liquid biopsies of 6 melanoma patients (P1-P6). FIG. 2A is a photographic representation of the localization of PD-L1 and CSV in patients P1-P6 at 1 week, and FIG. 2B depicts the TCFI of CSV for samples taken at 1 week, TNFI of PD-L1 for samples taken at 1 week, and the Fn/c of PD-L1 for samples taken at 1 week (n≥5-10 individual cells per sample). Data is shown as mean±SE. Representative images for each condition are shown.

FIG. 3 presents the localization of PD-L1 in breast cancer cells. FIG. 3A presents the TNFI and TCFI of PD-L1 in MDA-MB-231 cells (MDA) and stimulated (MCF7ST) and non-stimulated (MCF7NS) MCF7 cells. FIG. 3B depicts the TNFI of PD-L1 in MDA-MB-231 mouse xenograft cells treated with 60 mg/kg abraxane or 10 mg/kg docetaxel (Dox) for 35 days. The tumor volume prior to excision is also presented. FIG. 3C is a photographic and graphical representation of localization of PD-L1, H3K27Ac, H3K4me3 and H3K9me3 in MDA-MB-231 cells. TNFI and Pearson's co-efficient correlation (PCC(r)) are presented. Data are shown as mean +SE. n≥20 individual cells per sample; NS=non-stimulated; ST=stimulated with PMA. Representative images for each condition are shown.

FIG. 4 presents a schematic representation of the PD-L1 wild type plasmid and the PD-L1 with a K263Q mutation plasmid (Mut1 plasmid). Lysine 263 in the wild type plasmid and glutamine 263 in the Mut1 plasmid are underlined.

FIG. 5 is a photographic (FIG. 5A) and graphical (FIGS. 5B to 5E) representation of localization of PD-L1 and CSV in stimulated and non-stimulated MCF7 cells transfected with an empty vector (VO), the PD-L1 wild type plasmid (PDL1-WT), and the PD-L1 Mut1 plasmid (PDL1-Mut1). FIG. 5B depicts the TCFI of CSV, FIG. 5C depicts the TNFI of PD-L1, FIG. 5D depicts the TCFI of PD-L1, and FIG. 5E depicts the Fn/c of PD-L1 (n≥5-10 individual cells per sample; NS=non-stimulated; ST=stimulated with PMA). Data is shown as mean±SE. Representative images for each condition are shown. ****=p-value of ≤0.0001; ***=p-value of ≤0.001; **=p-value of ≤0.01; *=p-value of ≤0.05; ns=p-value of >0.05.

FIG. 6 is a photographic (FIG. 6A) and graphical (FIGS. 6B to 6H) representation of localization of epidermal growth factor receptor (EGFR), CD133 and SNAI1 in stimulated and non-stimulated MCF7 cells transfected with an empty vector (VO), the PD-L1 wild type plasmid (PDL1-WT), and the PD-L1 Mut1 plasmid (PDL1-Mut1). FIG. 6B depicts the TNFI of EGFR, FIG. 6C depicts the TCFI of EGFR, FIG. 6D depicts the Fn/c of EGFR, FIG. 6E depicts the TNFI of SNAI1, FIG. 6F depicts the TCFI of SNAI1, FIG. 6G depicts the Fn/c of SNAI1, and FIG. 6H depicts the TCFI of CD133 (n≥5-10 individual cells per sample; NS=non-stimulated; ST=stimulated with PMA). Data is shown as mean±SE. Representative images for each condition are shown. ****=p-value of ≤0.0001; ***=p-value of ≤0.001; **=p-value of ≤0.01; *=p-value of ≤0.05; ns=p-value of >0.05.

FIG. 7 is a graphical representation of the proliferation of MCF7 cells transfected with an empty vector (Vector only), the PD-L1 wild type plasmid (WT), and the PD-L1 Mut1 plasmid (Mut-1) incubated for 24 or 48 hours with the plasmid and treated for 2, 3 or 4 hours with the WST-1 reagent. ****=p-value of ≤0.0001; ***=p-value of ≤0.001; **=p-value of ≤0.01; *=p-value of ≤0.05; ns=p-value of >0.05.

FIG. 8 is a photographic and graphical representation of the localization of PD-L1, PD-L1 trimethylated at lysine 263 (PDL1me3; ‘trimethylated PD-L1’) and PD-L1 acetylated at lysine 263 (PDL1(Ac); ‘acetylated PD-L1’) in MDA-MB-231 cells (FIG. 8A) and metastatic melanoma and metastatic breast cancer patient cells (FIGS. 8B and 8C). FIG. 8A presents the nuclear localization (Fn/c) of trimethylated and acetylated PD-L1 and native PD-L1 in MDA-MB-231 cells. FIG. 8B presents the localization of trimethylated PD-L1 in CTCs isolated from metastatic melanoma patients which respond to immunotherapy (responder), CTCs isolated from metastatic melanoma patients which display primary resistance to immunotherapy (primary resistance), CTCs isolated from metastatic breast cancer patients (MBC CTC S2) and MDA-MB-231 cells. TCFI of trimethylated PD-L1 is presented. CSV (cell surface vimentin) is used as a control. FIG. 8C depicts the localization of acetylated PD-L1 in CTCs isolated from metastatic melanoma patients which respond to immunotherapy (responder), CTCs isolated from metastatic melanoma patients which display secondary resistance to immunotherapy (2nd resistance), CTCs isolated from metastatic breast cancer patients (MBC CTC S1) and MDA-MB-231 cells. TNFI of acetylated PD-L1 is presented. CSV (cell surface vimentin) is used as a control. Data is shown as mean±SE (n=20 cells/sample). Representative images for each condition are shown. ****=p-value of ≤0.0001; ***=p-value of ≤0.001; **=p-value of ≤0.01; *=p-value of ≤0.05; ns=p-value of >0.05.

FIG. 9 presents the effect of P1, P2 and P3 (referred to as PDL1-P1, PDL1-P2 and PDL1-P3, respectively) on the localization of PD-L1 and acetylated PD-L1 in MDA-MB-231 cells. FIG. 9A is a photographic and graphical representation of the localization of PD-L1 in MDA-MB-231 cells in response to treatment with P1, P2 and P3. FIG. 9B is a photographic and graphical representation of the localization of acetylated PD-L1 in MDA-MB-231 cells in response to treatment with P1, P2 and P3. TN FI, TCFI and Fn/c are presented. Data is shown as mean±SE (n=20 cells/sample). Representative images for each condition are shown. ****=p-value of ≤0.0001; ***=p-value of ≤0.001; **=p-value of ≤0.01; *=p-value of ≤0.05; ns=p-value of >0.05.

FIG. 10 is a photographic and graphical representation of the effect of P4 (referred to as PDL1-P4) on the localization of PD-L1 and acetylated PD-L1 in MDA-MB-231 cells. FIG. 10A presents the localization of acetylated PD-L1 and FIG. 10B presents the localization of PD-L1 in response to treatment with P4. TNFI, TCFI and Fn/c are presented. Data is shown as mean±SE (n=20 cells/sample). Representative images for each condition are shown. ****=p-value of ≤0.0001; ***=p-value of ≤0.001; **=p-value of ≤0.01; *=p-value of ≤0.05; ns=p-value of >0.05.

FIG. 11 presents the effect of P1, P2 and P3 on the cancer stem cell phenotype (CD44^{high}/CD24^{low}) in MDA-MB-231 cells. FIG. 11A depicts the FACS analysis of cells treated with P1, FIG. 11B depicts the FACS analysis of cells treated with P2, FIG. 11C depicts the FACS analysis of cells treated with P3, FIG. 11D is a graphical representation of the FACS analysis of cells treated with P1 (referred to as peptide 1), FIG. 11E is a graphical representation of the FACS analysis of cells treated with P2 (referred to as peptide 2), and FIG. 11F is a graphical representation of the FACS analysis of cells treated with P3 (referred to as peptide 3).

FIG. 12 is a graphical representation of the effect of P1 (referred to as peptide 1; FIG. 12A), P2 (referred to as peptide 2; FIG. 12B) and P3 (referred to as peptide 3; FIG. 12C) on MDA-MB-231 cell proliferation. Cells were treated with peptide for 72 hours, followed by incubation for 2 hours with the WST-1 reagent.

FIG. 13 is a photographic (FIG. 13A) and graphical (FIGS. 13B to 13D) representation of localization of CSV, PD-L1 and SNAI1 in MDA-MB-231 cells treated with P1 (PDL1-P1), P2 (PDL1-P2) and P3 (PDL1-P3). FIG. 13B depicts the TCFI of CSV, FIG. 13C depicts the TNFI of PD-L1, and FIG. 13D depicts the TNFI of SNAI1 (n≥20 individual cells per sample). Data is shown as mean±SE. Representative images for each condition are shown. ****=p-value of ≤0.0001; ***=p-value of ≤0.001; **=p-value of ≤0.01; *=p-value of ≤0.05; ns=p-value of >0.05.

FIG. 14 is a photographic (FIG. 14A) and graphical (FIGS. 14B to 14D) representation of localization of CSV, EGFR and SNAI1 in MDA-MB-231 cells treated with P1 (PDL1-P1), P2 (PDL1-P2) and P3 (PDL1-P3). FIG. 14B depicts the TCFI of CSV, FIG. 14C depicts the TNFI of EGFR, and FIG. 14D depicts the TNFI of SNAI1 (n≥20 individual cells per sample). Data is shown as mean±SE. Representative images for each condition are shown. ****=p-value of ≤0.0001; ***=p-value of ≤0.001; **=p-value of ≤0.01; *=p-value of ≤0.05; ns=p-value of >0.05.

FIG. 15 is a photographic and graphical representation of the localization of EHMT2, DMNTI and SETDB1 in MDA-MB-231 cells treated with P1, P2, P3 or P4. TNFI is presented. Data is shown as mean±SE. Representative images for each condition are shown. n≥20 individual cells per sample. ****=p-value of ≤0.0001; ***=p-value of ≤0.001; **=p-value of ≤0.01; *=p-value of ≤0.05; ns=p-value of >0.05.

FIG. 16 is a photographic and graphical representation of the localization of H3K9me3 (FIG. 16A) and 5-methylcytosine (FIG. 16B) in MDA-MB-231 cells treated with vehicle (control) or P3. ABCB5 is included as a marker for chemo-resistance. TNFI and TFI are presented. Data is shown as mean±SE (n=20 cells/sample). Representative images for each condition are shown. ****=p-value of ≤0.0001; ***=p-value of ≤0.001; **=p-value of ≤0.01; *=p-value of ≤0.05; ns=p-value of >0.05.

FIG. 17 is a photographic and graphical representation of the localization of acetylated PD-L1 and p300 in CTCs isolated from metastatic melanoma patients which respond to immunotherapy treatment (responder), or which display primary or secondary resistance to immunotherapy treatment (resistant). TNFI and PCC(r) are presented. Data is shown as mean±SE (n=20 cells/sample). Representative images for each condition are shown. ****=p-value of ≤0.0001; ***=p-value of ≤0.001; **=p-value of ≤0.01; *=p-value of ≤0.05; ns=p-value of >0.05.

FIG. 18 is a photographic and graphical representation of the localization of acetylated PD-L1 and p300 in matched naïve permeabilized MDA-MB-231, MCF7, T-47D and 4T1 (4T1 Group A) cells, docetaxel resistant permeabilized MDA-MB-231 (MDA-MB-231 TXT50), MCF7 (MCF7 TXT50) and T-47D (T-47D TXT50) cells, and abraxane resistant permebilized 4T1 (4T1 Group B) cells. TNFI and PCC(r) are presented. Data is shown as mean±SE (n=20 cells/sample). Representative images for each condition are shown. ****=p-value of ≤0.0001; ***=p-value of ≤0.001; **=p-value of ≤0.01; *=p-value of ≤0.05; ns=p-value of >0.05.

FIG. 19 is a photographic and graphical representation of the localization of acetylated PD-L1 and p300 in MDA-MB-231 cells treated with P1 (PDL1-P1), P2 (PDL1-P2), P3 (PDL1-P3), P4 (PDL1-P4) or vehicle (control). TNFI and PCC(r) are presented. Data is shown as mean±SE (n=20 cells/sample). Representative images for each condition are shown. ****=p-value of ≤0.0001; ***=p-value of ≤0.001; **=p-value of ≤0.01; *=p-value of ≤0.05; ns=p-value of >0.05.

FIG. 20 is a photographic (FIG. 20A) and graphical (FIG. 20B) representation of localization of PD-1 in Jurkat T-cells or OT1 derived T-cells. FIG. 20B depicts the TNFI of PD-1 (n≥20 individual cells per sample). Data is shown as mean±SE. Representative images for each condition are shown. D0=day zero; D1=day one; D6=day six; NS=non-stimulated; ST=stimulated with PMA; RST=restimulated with PMA; SW=stimulated with PMA and washed; naïve=OT1 cells were obtained from naïve T-cells; E1, E2 or E3=OT1 cells were isolated from effector virus specific T-cells; ****=p-value of ≤0.0001; ***=p-value of ≤0.001; **=p-value of ≤0.01; *=p-value of ≤0.05; ns=p-value of >0.05.

FIG. 21 is a photographic (FIG. 21A) and graphical (FIGS. 21B to 21D) representation of localization of PD-1 in Jurkat T-cells treated with P1 (PEP1), P2 (PEP2) and P3 (PEP3). FIG. 21B depicts the TNFI of PD-1, FIG. 21C depicts the TCFI of PD-1, and FIG. 21D depicts the Fn/c of PD-1 (n≥20 individual cells per sample). Data is shown as mean±SE. Representative images for each condition are shown. ****=p-value of ≤0.0001; ***=p-value of ≤0.001; **=p-value of ≤0.01; *=p-value of ≤0.05; ns=p-value of >0.05.

FIG. 22 is a photographic (FIG. 22A) and graphical (FIGS. 22B to 22D) representation of localization of PD-L1, PD-L2 and CSV in MDA-MB-231 cells treated with P1 (PDL1-P1), P2 (PDL1-P2) and P3 (PDL1-P3). FIG. 22B depicts the TNFI of PD-L2, FIG. 22C depicts the TNFI of PD-L1, and FIG. 22D depicts the TCFI of CSV (n≥20 individual cells per sample). Data is shown as mean±SE. Representative images for each condition are shown. ****=p-value of ≤0.0001; ***=p-value of ≤0.001; **=p-value of ≤0.01; *=p-value of ≤0.05; ns=p-value of >0.05.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

The term “acetylation site” is used herein to refer to any amino acid sequence that may be acetylated, for example, by an acetyltransferase; especially a histone acetyltransferase, non-limiting examples of which include GCNS, Hat1, ATF-2, Tip60, MOZ, MORF, HBO1, p300, CBP, SRC-1, ACTR, TIF-2, SRC-3, TAF1, TFIIIC and/or CLOCK, most especially p300. The term “acetylation site” refers to a sequence comprising an acetylation substrate, such as a lysine residue, and surrounding and/or proximal amino acid residues which may be involved in substrate recognition by an enzyme, such as an acetyltransferase. The acetylation site may be an amino acid sequence of any suitable length, such as, 5, 6, 7, 8, 9, 10, 11, 12, 13, 14, 15, 16, 17, 18, 19, 20, 21, 22 or greater than 22 residues in length, preferably 14, 15, 16, 17, 18, 19, 20 or 21 residues in length.

The term “agent” includes a compound that induces a desired pharmacological and/or physiological effect. The term also encompasses pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to small molecules, proteinaceous molecules such as peptides, polypeptides and proteins as well as compositions comprising them and genetic molecules such as RNA, DNA and mimetics and chemical analogs thereof as well as cellular agents.

As used herein, the term “and/or” refers to and encompasses any and all possible combinations of one or more of the associated listed items, as well as the lack of combinations when interpreted in the alternative (or).

The term “cancer stem cell” (CSC) refers to a cell that has tumor-initiating and tumor-sustaining capacity, including the ability to extensively proliferate, form new tumors and maintain cancer development, i.e. cells with indefinite proliferative potential that drive the formation and growth of tumors. CSCs are biologically distinct from the bulk tumor cells and possess characteristics associated with stem cells, specifically the ability to self renew and to propagate and give rise to all cell types found in a particular cancer sample. The term “cancer stem cell” includes both gene alteration in stem cells (SCs) and gene alteration in a cell which becomes a CSC. In specific embodiments, the CSCs are breast CSCs, which are suitably CD24+ CD44+, illustrative examples of which include CD44^{high} CD24^{low}.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. Thus, the use of the term “comprising” and the like indicates that the listed integers are required or mandatory, but that other integers are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. In specific embodiments, the term “consisting essentially of”, in the context of a specific amino acid sequence disclosed herein, includes within its scope about 1 to about 50 optional amino acids (and all integer optional amino acids in between) upstream of the specific amino acid sequence and/or about 1 to about 50 optional amino acids (and all integer optional amino acids in between) downstream of the specific amino acid sequence.

By “corresponds to” or “corresponding to” is meant a sequence, such as a nucleic acid or amino acid sequence, that displays substantial sequence identity to a reference sequence (e.g. at least about 60, 61, 62, 63, 64, 65, 66, 67, 68, 69, 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence identity to all or a portion of the reference nucleic acid sequence) or an amino acid sequence that displays substantial sequence similarity or identity to a reference amino acid sequence (e.g. at least 60, 61, 62, 63, 64, 65, 66, 67, 68, 69, 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to all or a portion of the reference amino acid sequence).

By “derivative” is meant a molecule, such as a polypeptide, that has been derived from the basic molecule by modification, for example by conjugation or complexing with other chemical moieties or by post-translational modification techniques as would be understood in the art. The term “derivative” also includes within its scope alterations that have been made to a parent sequence including additions or deletions that provide for functionally equivalent molecules.

As used herein, the term “dosage unit form” refers to physically discrete units suited as unitary dosages for the subject to be treated, each unit containing a predetermined quantity of active material calculated to produce the desired therapeutic effect in association with the required pharmaceutically acceptable vehicle.

By “effective amount”, in the context of treating or preventing a condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

As used herein, the term “epithelial-to-mesenchymal transition” (EMT) refers to the conversion from an epithelial cell to a mesenchymal phenotype, which is a normal process of embryonic development. EMT is also the process whereby injured epithelial cells that function as ion and fluid transporters become matrix remodeling mesenchymal cells, in carcinomas, this transformation typically results in altered cell morphology, the expression of mesenchymal proteins and increased invasiveness. The criteria for defining EMT in vitro involve the loss of epithelial cell polarity, the separation into individual cells and subsequent dispersion after the acquisition of cell motility (refer to Vincent-Salomon and Thiery (2003), Breast Cancer Res., 5(2): 101-6). Classes of molecules that change in expression, distribution and/or function during EMT, and that are causally involved, include growth factors (e.g. transforming growth factor (TGF)-β, wnts), transcription factors (e.g. SNAI, SMAD, LEF and nuclear β-catenin), molecules of the cell-to-cell adhesion axis (cadherins, catenins), cytoskeletal modulators (Rho family) and extracellular proteases (matrix metalloproteinases, plasminogen activators) (refer to Thompson and Newgreen, Cancer Res. 2005; 65(14):5991-5).

As used herein, the term “epithelium” refers to the covering of internal and external surfaces of the body, including the lining of vessels and other small cavities. It consists of a collection of epithelial cells forming a relatively thin sheet or layer due to the constituent cells being mutually and extensively adherent laterally by cell-to-cell junctions. The layer is polarized and has apical and basal sides. Despite the tight regimentation of the epithelial cells, the epithelium does have some plasticity and cells in an epithelial layer can alter shape, such as change from flat to columnar or pinch in at one end and expand at the other. However, these tend to occur in cell groups rather than individually (refer to Thompson and Newgreen, Cancer Res. 2005; 65(14):5991-5).

The term “expression” refers the biosynthesis of a gene product. For example, in the case of a coding sequence, expression involves transcription of the coding sequence into mRNA and translation of mRNA into one or more polypeptides. Conversely, expression of a non-coding sequence involves transcription of the non-coding sequence into a transcript only. The term “expression” is also used herein to refer to the presence of a protein or molecule in a particular location and, thus, may be used interchangeably with “localization”.

By “expression vector” is meant any genetic element capable of directing the transcription of a polynucleotide contained within the vector and suitably the synthesis of a peptide or polypeptide encoded by the polynucleotide. Such expression vectors are known to practitioners in the art.

The term “high”, as used herein, refers to a measure that is greater than normal, greater than a standard such as a predetermined measure or a subgroup measure or that is relatively greater than another subgroup measure. For example, CD44^{high} refers to a measure of CD44 that is greater than a normal CD44 measure. Consequently, “CD44^{high}” always corresponds to, at the least, detectable CD44 in a relevant part of a subject's body or a relevant sample from a subject's body. A normal measure may be determined according to any method available to one skilled in the art. The term “high” may also refer to a measure that is equal to or greater than a predetermined measure, such as a predetermined cutoff. If a subject is not “high” for a particular marker, it is “low” for that marker. In general, the cut-off used for determining whether a subject is “high” or “low” should be selected such that the division becomes clinically relevant.

The term “hormone receptor negative (HR−) tumor” means a tumor that does not express a receptor for a hormone that stimulates the proliferation, survival or viability of the tumor above a certain threshold as determined by standard methods (e.g. immunohistochemical staining of nuclei in the patients biological samples). The threshold may be measured, for example, using an Allred score or gene expression. See, e.g. Harvey et al. (1999, J Clin Oncol, 17: 1474-1481) and Badve et al. (2008, J Clin Oncol, 26(15): 2473-2481). In some embodiments, the tumor does not express an estrogen receptor (ER−) and/or a progesterone receptor (PR−).

The term “hormone receptor positive (HR+) tumor” means a tumor that expresses a receptor for a hormone that stimulates the proliferation, survival or viability of the tumor above a certain threshold as determined by standard methods (e.g. immunohistochemical staining of nuclei in the patients biological samples). The threshold may be measured, for example, using an Allred score or gene expression. See, e.g., Harvey et al. (1999, J Clin Oncol, 17: 1474-1481) and Badve et al. (2008, J Clin Oncol, 26(15): 2473-2481). In some embodiments, the tumor expresses an estrogen receptor (ER) and/or a progesterone receptor (PR).

The term “host cell” includes an individual cell or cell culture which can be or has been a recipient of any recombinant vector(s) or isolated polynucleotide of the invention. Host cells include progeny of a single host cell and the progeny may not necessarily be completely identical (in morphology or in total DNA complement) to the original parent cell due to natural, accidental or deliberate mutation and/or change. A host cell includes cells transfected or infected in vivo or in vitro with a recombinant vector or a polynucleotide of the invention. A host cell which comprises a recombinant vector of the invention is a recombinant host cell.

“Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances as known to those of skill in the art.

The term “inhibitor” as used herein refers to an agent that decreases or inhibits at least one function or biological activity of a target molecule.

As used herein, the term “isolated” refers to material that is substantially or essentially free from components that normally accompany it in its native state. For example, an “isolated proteinaceous molecule” refers to in vitro isolation and/or purification of a proteinaceous molecule from its natural cellular environment and from association with other components of the cell. “Substantially free” means that a preparation of proteinaceous molecule is at least 10, 15, 20, 25, 30, 35, 40, 45, 50, 55, 60, 65, 70, 75, 80, 85, 90, 91, 92, 93, 94, 95, 96, 97, 98 or 99% pure. In a preferred embodiment, the preparation of proteinaceous molecule has less than about 30, 25, 20, 15, 10, 9, 8, 7 , 6, 5, 4, 3, 2 or 1% (by dry weight), of molecules that are not the subject of this invention (also referred to herein as “contaminating molecules”). When the proteinaceous molecule is recombinantly produced, it is also desirably substantially free of culture medium, i.e., culture medium represents less than about 20, 15, 10, 5, 4, 3, 2 or 1% of the volume of the preparation. The invention includes isolated or purified preparations of at least 0.01, 0.1, 1.0, and 10 milligrams in dry weight.

As used herein, the term “mesenchymal-to-epithelial transition” (MET) is a reversible biological process that involves the transition from motile, multipolar or spindle-shaped mesenchymal cells to planar arrays of polarized cells called epithelia. MET is the reverse process of EMT. METs occur in normal development, cancer metastasis and induced pluripotent stem cell reprogramming.

As used herein, the term “mesenchyme” refers to the part of the embryonic mesoderm, consisting of loosely packed, unspecialized cells set in a gelatinous ground substance, from which connective tissue, bone, cartilage and the circulatory and lymphatic systems develop.

Mesenchyme is a collection of cells which form a relatively diffuse tissue network. Mesenchyme is not a complete cellular layer and the cells typically have only points on their surface engaged in adhesion to their neighbors. These adhesions may also involve cadherin association (see Thompson and Newgreen (2005), Cancer Res., 65(14): 5991-5).

By “modulating” is meant increasing or decreasing, either directly or indirectly, the level or functional activity of a target molecule. For example, an agent may indirectly modulate the level/activity by interacting with a molecule other than the target molecule. In this regard, indirect modulation of a gene encoding a target polypeptide includes within its scope modulation of the expression of a first nucleic acid molecule, wherein an expression product of the first nucleic acid molecule modulates the expression of a nucleic acid molecule encoding the target polypeptide.

As used herein, the terms “overexpress”, “overexpression”, “overexpressing” or “overexpressed” interchangeably refer to a gene (e.g. PD-1 gene, PD-L1 gene or PD-L2 gene) that is transcribed or translated at a detectably greater level, usually in a cancer cell, in comparison to a normal cell. Overexpression, therefore, refers to both overexpression of protein and RNA (due to increased transcription, post transcriptional processing, translation, post translational processing, altered stability and altered protein degradation), as well as local overexpression due to altered protein traffic patterns (increased nuclear localization) and augmented functional activity. Overexpression can also be by 10%, 20%, 30%, 40%, 50%, 60%, 70%, 80%, 90% or more in comparison to a normal cell or comparison cell (e.g. a breast cell).

The term “operably linked” as used herein means placing a structural gene under the regulatory control of a regulatory element including, but not limited to, a promoter, which then controls the transcription and optionally translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting, i.e. the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting, i.e. the genes from which it is derived.

As used herein, the terms “PD-1-overexpressing cell”, “PD-L1-overexpressing cell” and “PD-L2-overexpressing cell” refer to a vertebrate cell, particularly a mammalian or avian (bird) cell, especially a mammalian cell, that expresses PD-1, PD-L1 or PD-L2 at a detectably greater level than a normal cell. The cell may be a vertebrate cell, such as a primate cell; an avian (bird) cell; a livestock animal cell such as a sheep cell, cow cell, horse cell, deer cell, donkey cell and pig cell; a laboratory test animal cell such as a rabbit cell, mouse cell, rat cell, guinea pig cell and hamster cell; a companion animal cell such as a cat cell and dog cell; and a captive wild animal cell such as a fox cell, deer cell and dingo cell. In particular embodiments, the PD-1, PD-L1 or PD-L2 overexpressing cell is a human cell. In specific embodiments, the PD-1, PD-L1 or PD-L2 overexpressing cell is a cancer stem cell or a non-cancer stem cell tumor cell; preferably a cancer stem cell tumor cell. Overexpression can also be by 10%, 20%, 30%, 40%, 50%, 60%, 70%, 80%, 90% or more in comparison to a normal cell or comparison cell (e.g. a breast cell).

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, transfection agents and the like.

Similarly, a “pharmacologically acceptable” salt, ester, amide, prodrug or derivative of a compound as provided herein is a salt, ester, amide, prodrug or derivative that this not biologically or otherwise undesirable.

As used herein, the terms “polypeptide”, “proteinaceous molecule”, “peptide” and “protein” are used interchangeably to refer to a polymer of amino acid residues and to variants and synthetic analogues of the same. Thus, these terms apply to amino acid polymers in which one or more amino acid residues is a synthetic non-naturally-occurring amino acid, such as a chemical analogue of a corresponding naturally-occurring amino acid, as well as to naturally-occurring amino acid polymers. These terms do not exclude modifications, for example, glycosylations, acetylations, phosphorylations and the like. Soluble forms of the subject proteinaceous molecules are particularly useful. Included within the definition are, for example, polypeptides containing one or more analogues of an amino acid including, for example, unnatural amino acids or polypeptides with substituted linkages.

As used herein, the terms “prevent”, “prevented” or “preventing”, refer to a prophylactic treatment which increases the resistance of a subject to developing the disease or condition or, in other words, decreases the likelihood that the subject will develop the disease or condition as well as a treatment after the disease or condition has begun in order to reduce or eliminate it altogether or prevent it from becoming worse. These terms also include within their scope preventing the disease or condition from occurring in a subject which may be predisposed to the disease or condition but has not yet been diagnosed as having it.

The terms “reduce”, “inhibit”, “suppress”, “decrease”, and grammatical equivalents when used in reference to the level of a substance and/or phenomenon in a first sample relative to a second sample, mean that the quantity of substance and/or phenomenon in the first sample is lower than in the second sample by any amount that is statistically significant using any art-accepted statistical method of analysis. In one embodiment, the reduction may be determined subjectively, for example when a patient refers to their subjective perception of disease symptoms, such as pain, fatigue, etc. In another embodiment, the reduction may be determined objectively, for example when the number of CSCs and/or non-CSC tumor cells in a sample from a patient is lower than in an earlier sample from the patient. In another embodiment, the quantity of substance and/or phenomenon in the first sample is at least 10% lower than the quantity of the same substance and/or phenomenon in a second sample. In another embodiment, the quantity of the substance and/or phenomenon in the first sample is at least 25% lower than the quantity of the same substance and/or phenomenon in a second sample. In yet another embodiment, the quantity of the substance and/or phenomenon in the first sample is at least 50% lower than the quantity of the same substance and/or phenomenon in a second sample. In a further embodiment, the quantity of the substance and/or phenomenon in the first sample is at least 75% lower than the quantity of the same substance and/or phenomenon in a second sample. In yet another embodiment, the quantity of the substance and/or phenomenon in the first sample is at least 90% lower than the quantity of the same substance and/or phenomenon in a second sample. Alternatively, a difference may be expressed as an “n-fold” difference.

As used herein, the terms “salts” and “prodrugs” include any pharmaceutically acceptable salt, ester, hydrate or any other compound which, upon administration to the recipient, is capable of providing (directly or indirectly) a proteinaceous molecule of the invention, or an active metabolite or residue thereof. Suitable pharmaceutically acceptable salts include salts of pharmaceutically acceptable inorganic acids such as hydrochloric, sulfuric, phosphoric, nitric, carbonic, boric, sulfamic and hydrobromic acids, or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulfonic, toluenesulfonic, benzenesulfonic, salicylic, sulfanilic, aspartic, glutamic, edetic, stearic, palmitic, oleic, lauric, pantothenic, tannic, ascorbic and valeric acids. Base salts include, but are not limited to, those formed with pharmaceutically acceptable cations, such as sodium, potassium, lithium, calcium, magnesium, ammonium and alkylammonium. Also, basic nitrogen-containing groups may be quaternized with such agents as lower alkyl halides, such as methyl, ethyl, propyl and butyl chlorides, bromides and iodides; dialkyl sulfates such as dimethyl and diethyl sulfate; and others. However, it will be appreciated that non-pharmaceutically acceptable salts also fall within the scope of the invention since these may be useful in the preparation of pharmaceutically acceptable salts. The preparation of salts and prodrugs can be carried out by methods known in the art. For example, metal salts can be prepared by reaction of a compound of the invention with a metal hydroxide. An acid salt can be prepared by reacting an appropriate acid with a proteinaceous molecule of the invention.

The term “stringency” as used herein, refers to the temperature and ionic strength conditions, and presence or absence of certain organic solvents during hybridization and washing procedures. The higher the stringency, the higher will be the degree of complementarity between immobilized target nucleotide sequences and the labelled probe polynucleotide sequences that remain hybridized to the target after washing. The term “high stringency” refers to temperature and ionic conditions under which only nucleotide sequences having a high frequency of complementary bases will hybridize. The stringency required is nucleotide sequence dependent and depends upon the various components present during hybridization. Generally, stringent conditions are selected to be about 10 to 20° C. lower than the thermal melting point (Tₘ) for the specific sequence at a defined ionic strength and pH. The Tₘ is the temperature (under defined ionic strength and pH) at which 50% of a target sequence hybridizes to a complementary probe.

The term “subject” as used herein refers to a vertebrate subject, particularly a mammalian or avian (bird) subject, for whom therapy or prophylaxis is desired. Suitable subjects include, but are not limited to, primates; avians (birds); livestock animals such as sheep, cows, horses, deer, donkeys and pigs; laboratory test animals such as rabbits, mice, rats, guinea pigs and hamsters; companion animals such as cats and dogs; and captive wild animals such as foxes, deer and dingoes. In particular, the subject is a human. However, it will be understood that the aforementioned terms do not imply that symptoms are present.

As used herein, the terms “treatment”, “treating”, and the like, refer to obtaining a desired pharmacologic and/or physiologic effect. The effect may be therapeutic in terms of a partial or complete cure for a disease or condition and/or adverse effect attributable to the disease or condition. These terms also cover any treatment of a condition or disease in a mammal, particularly in a human, and include: (a) inhibiting the disease or condition, i.e. arresting its development; or (b) relieving the disease or condition, i.e. causing regression of the disease or condition.

As used herein, the term “tumor” refers to any neoplastic cell growth and proliferation, whether malignant or benign, and all pre-cancerous and cancerous cells and tissues. The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. The term “non-metastatic” refers to a cancer that is benign or that remains at the primary site and has not penetrated into the lymphatic or blood vessel system or to tissues other than the primary site. Generally, a non-metastatic cancer is any cancer that is a Stage 0, I or II cancer. By “early stage cancer” is meant a cancer that is not invasive or metastatic or is classified as a Stage 0, I or II cancer. The term “late stage cancer” generally refers to a Stage III or IV cancer, but can also refer to a Stage II cancer or a substage of a Stage II cancer. One skilled in the art will appreciate that the classification of a Stage II cancer as either an early stage cancer or a late stage cancer depends on the particular type of cancer. Illustrative examples of cancer include, but are not limited to, breast cancer, prostate cancer, ovarian cancer, cervical cancer, pancreatic cancer, colorectal cancer, lung cancer, hepatocellular cancer, gastric cancer, liver cancer, bladder cancer, cancer of the urinary tract, thyroid cancer, renal cancer (kidney cancer), carcinoma, retinoblastoma, melanoma, brain cancer, non-small cell lung cancer, squamous cell cancer of the head and neck, endometrial cancer, multiple myeloma, mesothelioma, rectal cancer and esophageal cancer. In an exemplary embodiment, the cancer is breast cancer or melanoma.

As used herein, the term “vector” refers to a polynucleotide molecule, suitably a DNA molecule derived, for example, from a plasmid, bacteriophage, yeast or virus, into which a polynucleotide can be inserted or cloned. A vector may contain one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e. a vector that exists as an extra-chromosomal entity, the replication of which is independent of chromosomal replication, e.g. a linear or closed circular plasmid, an extra-chromosomal element, a mini-chromosome or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably a viral or viral-derived vector, which is operably functional in fungi, bacterial or animal cells, preferably mammalian cells. Such vector may be derived from a poxvirus, an adenovirus or yeast. The vector can also include a selection marker such as an antibiotic resistance gene that can be used for selection of suitable transformants. Examples of such resistance genes are known to those of skill in the art and include the nptll gene that confers resistance to the antibiotics kanamycin and G418 (Geneticin®) and the hph gene which confers resistance to the antibiotic hygromycin B.

Each embodiment described herein is to be applied mutatis mutandis to each and every embodiment unless specifically stated otherwise.

### 2. Abbreviations

The following abbreviations are used throughout the specification:

K(Ac)=Nε-acetyl-L-lysine

Nle=Norleucine

Orn(Ac)=N_{δ}-acetyl-L-ornithine

K(Me₃)=Nε-trimethyl-L-lysine

Orn=Ornithine

### 3. Proteinaceous Molecules

The present invention is based, in part, on the identification that proteinaceous molecules corresponding to an acetylation site, such as a site of PD-L1, inhibit or reduce the nuclear localization of a polypeptide in which acetylation of an acetylation site increases nuclear localization of the polypeptide, such as an immune checkpoint protein, including PD-1, PD-L1 and/or PD-L2. In one or more embodiments, such proteinaceous molecules inhibit or decrease the formation, maintenance, and/or viability of cancer stem cell and non-cancer stem cell tumor cells, and/or inhibit EMT and/or induce MET of cancer stem cell tumor cells. Thus, the inventors have conceived that the proteinaceous molecules of the invention may be used for the treatment or prevention of cancer.

Accordingly, in one aspect of the invention, there is provided an isolated or purified proteinaceous molecule represented by Formula I:

Z₁X₁X₂X₃X₄FX₅X₆X₇X₈X₉X₁₀X₁₁X₁₂X₁₃X₁₄X₁₅X₁₆Z₂ (I)

wherein:
Z₁ and Z₂ are independently absent or are independently selected from at least one of a proteinaceous moiety comprising from about 1 to about 50 amino acid residues (and all integer residues in between), and a protecting moiety;
X₁ is absent or is selected from small amino acid residues including A, G, S, T and modified forms thereof, and hydrophobic amino acid residues including M, Nle, I, L, V, F, Y, W and modified forms thereof;
X₂ is selected from small amino acid residues including A, G, S, T and modified forms thereof, and charged amino acid residues including K, R, D, E and modified forms thereof;
X₃ is selected from any amino acid residue;
X₄ is selected from charged amino acid residues including K, R, D, E and modified forms thereof, and hydrophobic amino acid residues including M, Nle, I, L, V, F, Y, W and modified forms thereof;
X₅ is selected from any amino acid residue;
X₆ is selected from charged amino acid residues including K, R, D, E and modified forms thereof, and hydrophobic amino acid residues including M, Nle, I, L, V, F, Y, W and modified forms thereof;
X₇ is selected from charged amino acid residues including K, R, D, E and modified forms thereof, and hydrophobic amino acid residues including M, Nle, I, L, V, F, Y, W and modified forms thereof;
X₈ is selected from small amino acid residues including A, G, S, T and modified forms thereof, basic amino acid residues including K, R, Orn and modified forms thereof, and amino acid residues with an amide-containing side chain including N, Q, Orn(Ac), K(Ac) and modified forms thereof;
X₉ is selected from small amino acid residues including G, S, T and modified forms thereof, charged amino acid residues including K, R, D, E and modified forms thereof, and hydrophobic amino acid residues including M, Nle, I, L, V, F, Y, W and modified forms thereof;
X₁₀ is selected from any amino acid residue;
X₁₁ is selected from any amino acid residue;
X₁₂ is selected from charged amino acid residues including K, R, D, E and modified forms thereof, and hydrophobic amino acid residues including M, Nle, I, L, V, F, Y, W and modified forms thereof;
X₁₃ is selected from any amino acid residue;
X₁₄ is selected from any amino acid residue;
X₁₅ is selected from charged amino acid residues including K, R, D, E and modified forms thereof, and hydrophobic amino acid residues including M, Nle, I, L, V, F, Y, W and modified forms thereof; and
X₁₆ is selected from basic amino acid residues including K, R and modified forms thereof.

In some embodiments, Z₁ is absent. In other embodiments, Z₁ consists of 10, 9, 8, 7, 6, 5, 4, 3, 2 or 1 amino acid residues. In some embodiments the amino acid residues in Z₁ are independently selected from any amino acid residue.

In some embodiments, Z₂ is absent. In other embodiments, Z₂ consists of 10, 9, 8, 7, 6, 5, 4, 3, 2 or 1 amino acid residues. In some embodiments the amino acid residues in Z₂ are independently selected from any amino acid residue.

In some embodiments, X₁ is absent or is selected from L and A. In some embodiments, X₁ is A.

In some embodiments, X₂ is selected from small amino acid residues including A, G, S, T and modified forms thereof, and basic amino acid residues including K, R and modified forms thereof. In particular embodiments, X₂ is selected from T, A and K; especially A or K; most especially K.

In some embodiments, X₃ is selected from charged amino acid residues including K, R, D, E and modified forms thereof, and aromatic amino acid residues including F, Y, W and modified forms thereof. In particular embodiments, X₃ is selected from F, E and K; especially F.

In some embodiments, X₄ is selected from acidic amino acid residues including D, E and modified forms thereof, and hydrophobic amino acid residues including I, L, V, M, Nle and modified forms thereof. In particular embodiments, X₄ is selected from I, L and E; especially I.

In some embodiments, X₅ is selected from charged amino acid residues including K, R, D, E and modified forms thereof, and hydrophobic amino acid residues including M, Nle, I, L, V, F, Y, W and modified forms thereof. In particular embodiments, X₅ is selected from R, E and V; especially R or V; most especially V.

In some embodiments, X₆ is selected from L, E, K, F and V; especially L or F; most especially F.

In some embodiments, X₇ is selected from R, E and L; especially R or L; most especially L.

In some embodiments, X₈ is selected from small amino acid residues including A, G, S, T and modified forms thereof, and basic amino acid residues including K, R, Orn and modified forms thereof. In particular embodiments, X₈ is K or A; most especially A.

In some embodiments, X₉ is selected from G, acidic amino acid residues including D, E and modified forms thereof, and hydrophobic amino acid residues including M, Nle, I, L, V and modified forms thereof. In particular embodiments, X₉ is G, D or V; especially D or V.

In some embodiments, X₁₀ is selected from basic amino acid residues including K, R and modified forms thereof, and hydrophobic amino acid residues including M, Nle, I, L, V, F, Y, W and modified forms thereof. In particular embodiments, X₁₀ is R or V; especially R.

In some embodiments, X₁₁ is selected from small amino acid residues including A, G, S, T and modified forms thereof, hydrophobic amino acid residues including M, Nle, I, L, V, F, Y, W and modified forms thereof, and acidic amino acid residues including D, E and modified forms thereof. In particular embodiments, X₁₁ is selected from M, Nle, A and E; especially E or A; most especially A.

In some embodiments, X₁₂ is selected from hydrophobic amino acid residues including M, Nle, I, L, V, F, Y, W and modified forms thereof, and acidic amino acid residues including D, E and modified forms thereof. In particular embodiments, X₁₂ is M, Nle or E; especially E.

In some embodiments, X₁₃ is selected from small amino acid residues including A, G, S, T and modified forms thereof, hydrophobic amino acid residues including M, Nle, I, L, V, F, Y, W and modified forms thereof, and acidic amino acid residues including D, E and modified forms thereof. In particular embodiments, X₁₃ is A, D or V; especially A or V; most especially A.

In some embodiments, X₁₄ is selected from hydrophobic amino acid residues including M, Nle, I, L, V, F, Y, W and modified forms thereof, and basic amino acid residues including K, R and modified forms thereof. In particular embodiments, X₁₄ is V or K; especially K.

In some embodiments, X₁₅ is selected from basic amino acid residues including K, R and modified forms thereof, and aromatic amino acid residues including F, Y, W and modified forms thereof. In particular embodiments, X₁₅ is R, K or Y; especially K or Y; most especially K.

In some embodiments, X₁₆ is K or R; especially K.

In some embodiments, the isolated or purified proteinaceous molecule of Formula I comprises, consists or consists essentially of an amino acid sequence represented by any one of SEQ ID NO: 1-21:

[SEQ ID NO: 1]
LTFIFRLRKGRMMDVKK;
[SEQ ID NO: 2]
LTFIFRLRQGRMMDVKK;
[SEQ ID NO: 3]
LTFIFRLRK(Ac)GRMMDVKK;
[SEQ ID NO: 4]
ATFIFRLRKGRMMDVKK;
[SEQ ID NO: 5]
LKFIFRLRKGRMMDVKK;
[SEQ ID NO: 6]
AFIFRLRKGRMMDVKK;
[SEQ ID NO: 7]
LTFIFVLRKGRMMDVKK;
[SEQ ID NO: 8]
LTFIFRFRKGRMMDVKK;
[SEQ ID NO: 9]
LTFIFRLLKGRMMDVKK;
[SEQ ID NO: 10]
TFIFRLRAGRMMDVKK;
[SEQ ID NO: 11]
LTFIFRLRKDRMMDVKK;
[SEQ ID NO: 12]
LTFIFRLRKVRMMDVKK;
[SEQ ID NO: 13]
TFIFRLRKGRAMDVKK;
[SEQ ID NO: 14]
LTFIFRLRKGREMDVKK;
[SEQ ID NO: 15]
LTFIFRLRKGRM EDVKK;
[SEQ ID NO: 16]
TFIFRLRKGRMMAVKK;
[SEQ ID NO: 17]
LTFIFRLRKGRMMVVKK;
[SEQ ID NO: 18]
LTFIFRLRKGRMMDKKK;
[SEQ ID NO: 19]
TFIFRLRKGRMMDVKK;
[SEQ ID NO: 20]
TFIFRLRQGRMMDVKK;
or
[SEQ ID NO: 21]
TFIFRLRK(Ac)GRMMDVKK.

In some embodiments, the proteinaceous molecule of Formula I comprises, consists or consists essentially of an amino acid sequence represented by any one of SEQ ID NO: 1-18. In particular embodiments, the proteinaceous molecule of Formula I comprises, consists or consists essentially of an amino acid sequence represented by SEQ ID NO: 1, 4, 9, 10, 13, 16, 18 or 19.

In preferred embodiments, the proteinaceous molecule of Formula I has any one or more activities selected from the group consisting of: (i) increasing cell death; (ii) increasing MET; (iii) reducing or inhibiting EMT; (iv) inhibiting or reducing maintenance; (v) inhibiting or reducing proliferation; (vi) increasing differentiation; (vii) inhibiting or reducing formation; or (viii) reducing viability of a PD-1-, PD-L1- or PD-L2-overexpressing cell; especially a PD-L1-overexpressing cell. In some embodiments, the PD-1-, PD-L1- or PD-L2-overexpressing cell is a cancer stem cell or a non-cancer stem cell tumor cell; especially a cancer stem cell tumor cell.

In some embodiments, the proteinaceous molecule of Formula I has at least about 80%, 81%, 82%, 83%, 84%, 85%, 86%, 87%, 88%, 89%, 90%, 91%, 92%, 93%, 94%, 95%, 96%, 97%, 98%, or 99% sequence similarity to the amino acid sequence of SEQ ID NO: 1. In some embodiments, the proteinaceous molecule of Formula I has at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 1.

In preferred embodiments, the proteinaceous molecules of the invention do not comprise methionine. Methionine residues are prone to oxidation, which can result in reduced purity and loss of activity in solution. Suitable replacement amino acids for methionine residues may include, but are not limited to, valine, leucine, isoleucine, norleucine, norvaline, glycine or alanine; especially valine, leucine, isoleucine, norleucine or norvaline; most especially norleucine.

In some embodiments where the proteinaceous molecules of the invention comprise an N- and/or C-terminus, the proteinaceous molecules of the invention have a primary, secondary or tertiary amide, a hydrazide, a hydroxamide or a free-carboxyl group at the C-terminus and/or a primary amine or acetamide at the N-terminus. In some embodiments, the proteinaceous molecules of the invention are cyclic peptides and, thus, may not comprise N- and/or C-terminal amino acid residues.

The present invention also contemplates proteinaceous molecules that are variants of any one of SEQ ID NO: 1-21, especially any one of SEQ ID NO: 1-18. Such “variant” proteinaceous molecules include proteinaceous molecules derived from any one of SEQ ID NO: 1-21, especially any one of SEQ ID NO: 1-18 by deletion or addition of one or more amino acids to the N-terminal and/or C-terminal end of the proteinaceous molecule, deletion or addition of one or more amino acids at one or more sites in the proteinaceous molecule, or substitution of one or more amino acids at one or more sites in the proteinaceous molecule.

Variant proteinaceous molecules encompassed by the present invention are biologically active, that is, they continue to possess the desired biological activity of the native proteinaceous molecule. Such variants may result from, for example, genetic polymorphism or from human manipulation.

The proteinaceous molecules of any one of SEQ ID NO: 1-21, especially any one of SEQ ID NO: 1-18 may be altered in various ways, including amino acid substitutions, deletions, truncations and insertions. Methods for such manipulations are generally known in the art. For example, amino acid sequence variants of any one of SEQ ID NO: 1-21, especially any one of SEQ ID NO: 1-18 may be prepared by mutagenesis of nucleic acids encoding the amino acid sequence of any one of SEQ ID NO: 1-21, especially any one of SEQ ID NO: 1-18. Methods for mutagenesis and nucleotide sequence alterations are well known in the art. Refer to, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (“Molecular Biology of the Gene”, Fourth Edition, Benjamin/Cummings, Menlo Park, Calif., 1987) and the references cited therein. Guidance as to appropriate amino acid substitutions that do not affect biological activity of the proteinaceous molecule of interest may be found in the model of Dayhoff et al., (1978) Atlas of Protein Sequence and Structure (Natl. Biomed. Res. Found., Washington, D.C.). Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of the proteinaceous molecules of any one of SEQ ID NO: 1-21, especially any one of SEQ ID NO: 1-18. Recursive ensemble mutagenesis (REM), a technique which enhances the frequency of functional mutants in the libraries, can be used in combination with screening assays to identify active variants (Arkin and Yourvan (1992) Proc. Natl. Acad. Sci. USA 89: 7811-7815; Delgrave et al., (1993) Protein Engineering, 6: 327-331). Conservative substitutions, such as exchanging one amino acid with another having similar properties, may be desirable as discussed in more detail below.

Variant proteinaceous molecules of the invention may contain conservative amino acid substitutions at various locations along their sequence, as compared to a parent (e.g.

naturally-occurring or reference) amino acid sequence, such as any one of SEQ ID NO: 1-21, especially any one of SEQ ID NO: 1-18. A “conservative amino acid substitution” is one in which the amino acid residue is replaced with an amino acid residue having a similar side chain. Families of amino acid residues having similar side chains have been defined in the art as discussed in detail below.

Acidic: The residue has a negative charge due to loss of a proton at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with protons at physiological pH or within one or two pH units thereof (e.g. histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residue is charged at physiological pH and, therefore, includes amino acids having acidic or basic side chains, such as glutamic acid, aspartic acid, arginine, lysine and histidine.

Hydrophobic: The residue is not charged at physiological pH and the residue is repelled by aqueous solution so as to seek the inner positions in the conformation of a peptide in which it is contained when the peptide is in aqueous medium at physiological pH. Amino acids having a hydrophobic side chain include tyrosine, valine, isoleucine, leucine, methionine, norleucine, phenylalanine and tryptophan.

Neutral/polar: The residues are not charged at physiological pH but the residue is not sufficiently repelled by aqueous solutions so that it would seek inner positions in the conformation of a peptide in which it is contained when the peptide is in aqueous medium at physiological pH. Amino acids having a neutral/polar side chain include asparagine, glutamine, cysteine, histidine, serine and threonine.

This description also characterizes certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the a-amino group, as well as the a-carbon. Several amino acid similarity matrices (e.g. PAM120 matrix and PAM250 matrix as disclosed for example by Dayhoff et al., (1978), A model of evolutionary change in proteins. Matrices for determining distance relationships In M. O. Dayhoff, (ed.), Atlas of protein sequence and structure, Vol. 5, pp. 345-358, National Biomedical Research Foundation, Washington DC; and by Gonnet et al., (1992), Science, 256(5062): 1443-1445), however, include proline in the same group as glycine, serine, alanine and threonine. Accordingly, for the purposes of the present invention, proline is classified as a “small” amino acid.

The degree of attraction or repulsion required for classification as polar or non-polar is arbitrary and, therefore, amino acids specifically contemplated by the invention have been classified as one or the other. Most amino acids not specifically named can be classified on the basis of known behavior.

Amino acid residues can be further sub-classified as cyclic or non-cyclic, and aromatic or non-aromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small amino acid residues are, of course, always non-aromatic. Dependent on their structural properties, amino acid residues may fall in two or more classes. For the naturally-occurring protein amino acids, sub-classification according to this scheme is presented in Table 1.

TABLE 1
Amino Acid Sub-Classification

| Sub-classes | Amino Acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, Proline |
| Nonpolar/ | Alanine, Glycine, Isoleucine, Leucine, Methionine, |
| neutral | Phenylalanine, Proline, Tryptophan, Valine, |
| | Norleucine |
| Polar/neutral | Asparagine, Histidine, Glutamine, Cysteine, Serine, |
| | Threonine, Tyrosine |
| Polar/negative | Aspartic acid, Glutamic acid |
| Polar/positive | Lysine, Arginine |
| Polar/large | Asparagine, Glutamine |
| Polar | Arginine, Asparagine, Aspartic acid, Cysteine, |
| | Glutamic acid, Glutamine, Histidine, Lysine, Serine, |
| | Threonine, Tyrosine |
| Hydrophobic | Tyrosine, Valine, Isoleucine, Leucine, Methionine, |
| | Phenylalanine, Tryptophan, Norleucine |
| Aromatic | Tryptophan, Tyrosine, Phenylalanine |
| Residues that | Glycine and Proline |
| influence chain | |
| orientation | |

Conservative amino acid substitution also includes groupings based on side chains. For example, a group of amino acids having aliphatic side chains is glycine, alanine, valine, leucine, isoleucine and norleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartic acid with a glutamic acid, a threonine with a serine, or a similar replacement of an amino acid with a structurally related amino acid will not have a major effect on the properties of the resulting variant peptide of the invention. Whether an amino acid change results in a proteinaceous molecule that inhibits or reduces the nuclear localization of a nuclear localizable polypeptide, such as PD-1, PD-L1 and/or PD-L2, can readily be determined by assaying its activity. Conservative substitutions are shown in Table 2 under the heading of exemplary and preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE 2
Exemplary and Preferred Amino Acid Substitutions

| Original Residue | Exemplary Substitutions | Preferred Substitutions |
|---|---|---|
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Nle | Leu |
| Leu | Nle, Ile, Val, Met, Ala, Phe | Ile |
| Lys | Arg, Gln, Asn | Arg |
| Met | Leu, Ile, Phe, Nle | Nle |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Nle | Leu |

Alternatively, similar amino acids for making conservative substitutions can be grouped into three categories based on the identity of the side chains. The first group includes glutamic acid, aspartic acid, arginine, lysine and histidine, which all have charged side chains; the second group includes glycine, serine, threonine, cysteine, tyrosine, glutamine and asparagine; and the third group includes leucine, isoleucine, valine, alanine, proline, phenylalanine, tryptophan, methionine and norleucine, as described in Zubay, Biochemistry, third edition, Wm.C. Brown Publishers (1993).

Thus, a predicted non-essential amino acid residue in a proteinaceous molecule of the invention is typically replaced with another amino acid residue from the same side chain family. Alternatively, mutations can be introduced randomly along all or part of the coding sequence of a proteinaceous molecule of the invention, such as by saturation mutagenesis, and the resultant mutants can be screened for an activity of the parent polypeptide, as described for example herein, to identify mutants which retain that activity. Following mutagenesis of the coding sequences, the encoded proteinaceous molecule can be expressed recombinantly and its activity determined. A “non-essential” amino acid residue is a residue that can be altered from the wild-type sequence of an embodiment proteinaceous molecule of the invention without abolishing or substantially altering one or more of its activities. Suitably, the alteration does not substantially alter one of these activities, for example, the activity is at least 20%, 40%, 60%, 70% or 80% of that of the wild-type. By contrast, an “essential” amino acid residue is a residue that, when altered from the wild-type sequence of an embodiment proteinaceous molecule of the invention, results in abolition of an activity of the parent molecule such that less than 20% of the wild-type activity is present.

Accordingly, the present invention also contemplates variants of the proteinaceous molecules of any one of SEQ ID NO: 1-21, especially any one of SEQ ID NO: 1-18, wherein the variants are distinguished from the parent sequence by the addition, deletion, or substitution of one or more amino acid residues. In general, variants will display at least about 80%, 81%, 82%, 83%, 84%, 85%, 86%, 87%, 88%, 89%, 90%, 91%, 92%, 93%, 94%, 95%, 96%, 97%, 98%, 99% sequence similarity to a parent or reference proteinaceous molecule sequence as, for example, set forth in any one of SEQ ID NO: 1-21, especially any one of SEQ ID NO: 1-18, as determined by sequence alignment programs described elsewhere herein using default parameters. Desirably, variants will have at least 80%, 81%, 82%, 83%, 84%, 85%, 86%, 87%, 88%, 89%, 90%, 91%, 92%, 93%, 94%, 95%, 96%, 97%, 98%, 99% sequence identity to a parent or reference proteinaceous molecule sequence as, for example, set forth in any one of SEQ ID NO: 1-21, especially any one of SEQ ID NO: 1-18, as determined by sequence alignment programs described herein using default parameters. Variants of any one of SEQ ID NO: 1-21, especially any one of SEQ ID NO: 1-18, which fall within the scope of a variant proteinaceous molecule of the invention, may differ from the parent molecule generally by at least 1, but by less than 5, 4, 3, 2 or 1 amino acid residue(s). In some embodiments, a variant proteinaceous molecule of the invention differs from the corresponding sequence in any one of SEQ ID NO: 1-21, especially any one of SEQ ID NO: 1-18 by at least 1, but by less than 5, 4, 3, 2 or 1 amino acid residue(s). In some embodiments, the amino acid sequence of the variant proteinaceous molecule of the invention comprises the proteinaceous molecule of Formula I. In particular embodiments, the variant proteinaceous molecule of the invention inhibits or reduces nuclear localization of a nuclear localizable polypeptide, such as PD-1, PD-L1 and/or PD-L2.

If the sequence comparison requires alignment, the sequences are typically aligned for maximum similarity or identity. “Looped” out sequences from deletions or insertions, or mismatches, are generally considered differences. The differences are, suitably, differences or changes at a non-essential residue or a conservative substitution.

In some embodiments, calculations of sequence similarity or sequence identity between sequences are performed as follows:

To determine the percent identity of two amino acid sequences or of two nucleic acid sequences, the sequences are aligned for optimal comparison purposes (e.g. gaps can be introduced in one or both of a first and a second amino acid or nucleic acid sequence for optimal alignment and non-homologous sequences can be disregarded for comparison purposes). In some embodiments, the length of a reference sequence aligned for comparison purposes is at least 40%, more usually at least 50% or 60%, and even more usually at least 70%, 80%, 90% or 100% of the length of the reference sequence. The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. For amino acid sequence comparison, when a position in the first sequence is occupied by the same or similar amino acid residue (i.e. conservative substitution) at the corresponding position in the second sequence, then the molecules are similar at that position.

The percent identity between the two sequences is a function of the number of identical amino acid residues shared by the sequences at individual positions, taking into account the number of gaps and the length of each gap, which need to be introduced for optimal alignment of the two sequences. By contrast, the percent similarity between the two sequences is a function of the number of identical and similar amino acid residues shared by the sequences at individual positions, taking into account the number of gaps and the length of each gap, which need to be introduced for optimal alignment of the two sequences.

The comparison of sequences and determination of percent identity or percent similarity between sequences can be accomplished using a mathematical algorithm. In certain embodiments, the percent identity or similarity between amino acid sequences is determined using the Needleman and Wunsch, (1970, J. Mol. Biol., 48: 444-453) algorithm which has been incorporated into the GAP program in the GCG software package (Devereaux, et al. (1984) Nucleic Acids Research, 12: 387-395), using either a Blosum 62 matrix or a PAM250 matrix, and a gap weight of 16, 14, 12, 10, 8, 6, or 4 and a length weight of 1, 2, 3, 4, 5, or 6. In some embodiments, the percent identity or similarity between amino acid sequences can be determined using the algorithm of Meyers and Miller (1989, Cabios, 4: 11-17) which has been incorporated into the ALIGN program (version 2.0), using a PAM120 weight residue table, a gap length penalty of 12 and a gap penalty of 4.

The present invention also contemplates an isolated or purified proteinaceous molecule that is encoded by a polynucleotide sequence that hybridizes under stringency conditions as defined herein, especially under medium, high or very high stringency conditions, preferably under high or very high stringency conditions, to a polynucleotide sequence encoding the proteinaceous molecule of any one of SEQ ID NO: 1-21, especially any one of SEQ ID NO: 1-18, or the non-coding strand thereof. The invention also contemplates an isolated nucleic acid molecule comprising a polynucleotide sequence that hybridizes under stringency conditions as defined herein, especially under medium, high or very high stringency conditions, preferably under high or very high stringency conditions, to a polynucleotide sequence encoding the proteinaceous molecule of any one of any one of SEQ ID NO: 1-21, especially any one of SEQ ID NO: 1-18, or the non-coding strand thereof.

As used herein, the term “hybridizes under stringency conditions” describes conditions for hybridization and washing and may encompass low stringency, medium stringency, high stringency and very high stringency conditions.

Guidance for performing hybridization reactions can be found in Ausubel, et al. (1998) Current Protocols in Molecular Biology (John Wiley and Sons, Inc.), in particular sections 6.3.1-6.3.6. Both aqueous and non-aqueous methods can be used. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% sodium dodecyl sulfate (SDS) for hybridization at 65° C., and (i) 2× sodium chloride/sodium citrate (SSC), 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6×SSC at about 45° C., followed by two washes in 0.2×SSC, 0.1% SDS at least at 50° C. (the temperature of the washes can be increased to 55° C. for low stringency conditions). Medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

In some aspects of the present invention, there is provided an isolated or purified proteinaceous molecule of the invention that is encoded by a polynucleotide sequence that hybridizes under high stringency conditions to a polynucleotide sequence encoding the proteinaceous molecule of any one of SEQ ID NO: 1-21, especially any one of SEQ ID NO: 1-18, or the non-coding strand thereof. In certain embodiments, the isolated or purified proteinaceous molecule of the invention is encoded by a polynucleotide sequence that hybridizes under very high stringency conditions to a polynucleotide sequence encoding the proteinaceous molecule of any one of SEQ ID NO: 1-21, especially any one of SEQ ID NO: 1-18, or the non-coding strand thereof. One embodiment of very high stringency conditions includes hybridizing 0.5 M sodium phosphate, 7% SDS at 65° C., followed by one or more washes at 0.2×SSC, 1% SDS at 65° C. In some embodiments, the amino acid sequence of the variant proteinaceous molecule of the invention comprises the amino acid sequence of Formula I. In particular embodiments, the variant proteinaceous molecule of the invention inhibits or reduces nuclear localization of a nuclear localizable polypeptide, such as PD-1, PD-L1 and/or PD-L2.

Other stringency conditions are well known in the art and a person skilled in the art will recognize that various factors can be manipulated to optimize the specificity of the hybridization. Optimization of the stringency of the final washes can serve to ensure a high degree of hybridization. For detailed examples, see Ausubel, et al. (1998) Current Protocols in Molecular Biology (John Wiley and Sons, Inc.), in particular pages 2.10.1 to 2.10.16 and Sambrook, et al. (1989) Molecular Cloning: A Laboratory Manual (Cold Spring Harbour Press), in particular Sections 1.101 to 1.104.

While stringent washes are typically carried out at temperatures from about 42° C. to 68° C., a person skilled in the art will appreciate that other temperatures may be suitable for stringent conditions. Maximum hybridization rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel, et al. (1998) Current Protocols in Molecular Biology (John Wiley and Sons, Inc.) at page 2.10.8). In general, the Tₘ of a perfectly matched duplex of DNA may be predicted as an approximation by the formula:

Tₘ=81.5+16.6 (log₁₀M)+0.41 (% G+C)−0.63 (% formamide)−(600/length)

wherein: M is the concentration of Na⁺, preferably in the range of 0.01 M to 0.4 M; % G+C is the sum of guanosine and cytosine bases as a percentage of the total number of bases, within the range between 30% and 75% G+C; % formamide is the percent formamide concentration by volume; length is the number of base pairs in the DNA duplex. The Tₘ of a duplex DNA decreases by approximately 1° C. with every increase of 1% in the number of randomly mismatched base pairs. Washing is generally carried out at Tₘ-15° C. for high stringency, or Tₘ-30° C. for moderate stringency.

In one example of a hybridization procedure, a membrane (e.g. a nitrocellulose membrane or a nylon membrane) containing immobilized DNA is hybridized overnight at 42° C. in a hybridization buffer (50% deionized formamide, 5×SSC, 5× Denhardt's solution (0.1% ficoll, 0.1% polyvinylpyrrolidone and 0.1% BSA), 0.1% SDS and 200 mg/mL denatured salmon sperm DNA) containing labeled probe. The membrane is then subjected to two sequential medium stringency washes (i.e. 2×SSC, 0.1% SDS for 15 min at 45° C., followed by 2×SSC, 0.1% SDS for 15 min at 50° C.), followed by two sequential higher stringency washes (i.e. 0.2×SSC, 0.1% SDS for 12 min at 55° C. followed by 0.2×SSC and 0.1% SDS solution for 12 min at 65-68° C.

The proteinaceous molecules of the present invention also encompass a proteinaceous molecule comprising amino acids with modified side chains, incorporation of unnatural amino acid residues and/or their derivatives during peptide synthesis and the use of cross-linkers and other methods which impose conformational constraints on the proteinaceous molecules of the invention. Examples of side chain modifications include modifications of amino groups, such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with sodium borohydride; reductive alkylation by reaction with an aldehyde followed by reduction with sodium borohydride; and trinitrobenzylation of amino groups with 2,4,6-trinitrobenzene sulfonic acid (TNBS).

The carboxyl group may be modified by carbodiimide activation through O-acylisourea formation followed by subsequent derivatization, for example, to a corresponding amide.

The guanidine group of arginine residues may be modified by formation of heterocyclic condensation products with reagents such as 2,3-butanedione, phenylglyoxal and glyoxal.

Examples of incorporating unnatural amino acids and derivatives during peptide synthesis include, but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, N_{δ}-acetyl-L-ornithine, sarcosine, 2-thienyl alanine, Nε-acetyl-L-lysine, Nε-methyl-L-lysine, Nε-dimethyl-L-lysine, Nε-formyl-L-lysine and/or D-isomers of amino acids. A list of unnatural amino acids contemplated by the present invention is shown in Table 3.

TABLE 3
Exemplary Unnatural Amino Acids
Non-Conventional Amino Acids

| | |
|---|---|
| α-aminobutyric acid | L-N-methylalanine |
| α-amino-α-methylbutyrate | L-N-methylarginine |
| aminocyclopropane-carboxylate | L-N-methylasparagine |
| aminoisobutyric acid | L-N-methylaspartic acid |
| aminonorbornyl-carboxylate | L-N-methylcysteine |
| cyclohexylalanine | L-N-methylglutamine |
| cyclopentylalanine | L-N-methylglutamic acid |
| L-N-methylisoleucine | L-N-methylhistidine |
| D-alanine | L-N-methylleucine |
| D-arginine | L-N-methyllysine |
| D-aspartic acid | L-N-methylmethionine |
| D-cysteine | L-N-methylnorleucine |
| D-glutamate | L-N-methylnorvaline |
| D-glutamic acid | L-N-methylornithine |
| D-histidine | L-N-methylphenylalanine |
| D-isoleucine | L-N-methylproline |
| D-leucine | L-N-methylserine |
| D-lysine | L-N-methylthreonine |
| D-methionine | L-N-methyltryptophan |
| D-ornithine | L-N-methyltyrosine |
| D-phenylalanine | L-N-methylvaline |
| D-proline | L-N-methylethylglycine |
| D-serine | L-N-methyl-t-butylglycine |
| D-threonine | L-norleucine |
| D-tryptophan | L-norvaline |
| D-tyrosine | α-methyl-aminoisobutyrate |
| D-valine | α-methyl-γ-aminobutyrate |
| D-α-methylalanine | α-methylcyclohexylalanine |
| D-α-methylarginine | α-methylcylcopentylalanine |
| D-α-methylasparagine | α-methyl-α-naphthylalanine |
| D-α-methylaspartate | α-methylpenicillamine |
| D-α-methylcysteine | N-(4-aminobutyl)glycine |
| D-α-methylglutamine | N-(2-aminoethyl)glycine |
| D-α-methylhistidine | N-(3-aminopropyl)glycine |
| D-α-methylisoleucine | N-amino-α-methylbutyrate |
| D-α-methylleucine | α-napthylalanine |
| D-α-methyllysine | N-benzylglycine |
| D-α-methylmethionine | N-(2-carbamylethyl)glycine |
| D-α-methylornithine | N-(carbamylmethyl)glycine |
| D-α-methylphenylalanine | N-(2-carboxyethyl)glycine |
| D-α-methylproline | N-(carboxymethyl)glycine |
| D-α-methylserine | N-cyclobutylglycine |
| D-α-methylthreonine | N-cycloheptylglycine |
| D-α-methyltryptophan | N-cyclohexylglycine |
| D-α-methyltyrosine | N-cyclodecylglycine |
| L-α-methylleucine | L-α-methyllysine |
| L-α-methylnnethionine | L-α-methylnorleucine |
| L-α-methylnorvaline | L-α-methylornithine |
| L-α-methylphenylalanine | L-α-methylproline |
| L-α-methylserine | L-α-methylthreonine |
| L-α-methyltryptophan | L-α-methyltyrosine |
| L-α-methylvaline | L-N-methylhomophenylalanine |
| N-(N-(2,2-diphenylethyl) | N-(N-(3,3-diphenylpropyl) |
| carbamylmethyl)glycine | carbamylmethyl)glycine |
| 1-carboxy-1-(2,2-diphenyl-ethyl | L-selenocysteine |
| amino)cyclopropane | |
| D-selenocysteine | L-selenomethionine |
| L-telluromethionine | L-O-methyl homoserine |
| L-S-ethyl cysteine | L-ornithine |
| Nε-acetyl-L-lysine | Nε-methyl-L-lysine |
| Nε-dinnethyl-L-lysine | Nε-formyl-L-lysine |
| D-norleucine | D-norvaline |
| N_{δ}-acetyl-L-ornithine | |

In some embodiments, the proteinaceous molecule of the invention comprises at least one unnatural amino acid. In particular embodiments, the proteinaceous molecule of the invention comprises at least one norleucine residue.

Additional amino acids or other substituents may be added to the N- or C-termini, if present, of the proteinaceous molecules of the invention. For example, the proteinaceous molecules of the invention may form part of a longer sequence with additional amino acids added to either or both of the N- and C-termini.

For particular uses and methods of the invention, proteinaceous molecules with high levels of stability may be desired, for example, to increase the half-life of the proteinaceous molecule in a subject. Thus, in some embodiments, the proteinaceous molecules of the present invention comprise a stabilizing moiety or protecting moiety. The stabilizing moiety or protecting moiety may be coupled at any point on the peptide. Suitable stabilizing or protecting moieties include, but are not limited to, polyethylene glycol (PEG), a glycan or a capping moiety, including an acetyl group, pyroglutamate or an amino group. In preferred embodiments, the acetyl group and/or pyroglutamate are coupled to the N-terminal amino acid residue of the proteinaceous molecule. In particular embodiments, the N-terminus of the proteinaceous molecule is an acetamide. In preferred embodiments, the amino group is coupled to the C-terminal amino acid residue of the proteinaceous molecule. In particular embodiments, the proteinaceous molecule has a primary, secondary or tertiary amide, a hydrazide or a hydroxamide at the C-terminus; particularly a primary amide at the C-terminus. In preferred embodiments, the PEG is coupled to the N-terminal or C-terminal amino acid residue of the proteinaceous molecule or through the amino group of a lysine side-chain or other suitably modified side-chain, especially through the N-terminal amino acid residue such as through the amino group of the residue, or through the amino group of a lysine side-chain.

In preferred embodiments, the proteinaceous molecules of the present invention have a primary amide or a free carboxyl group (acid) at the C-terminus and a primary amine or acetamide at the N-terminus.

Although the proteinaceous molecules of the invention may inherently permeate membranes, membrane permeation may further be increased by the conjugation of a membrane permeating moiety to the proteinaceous molecule. Accordingly, in some embodiments, the proteinaceous molecules of the present invention comprise a membrane permeating moiety. The membrane permeating moiety may be coupled at any point on the proteinaceous molecule. Suitable membrane permeating moieties include lipid moieties, cholesterol and proteins, such as cell penetrating peptides and polycationic peptides; especially lipid moieties.

Suitable cell penetrating peptides may include the peptides described in, for example, US 20090047272, US 20150266935 and US 20130136742. Accordingly, suitable cell penetrating peptides may include, but are not limited to, basic poly(Arg) and poly(Lys) peptides and basic poly(Arg) and poly(Lys) peptides containing non-natural analogues of Arg and Lys residues such as YGRKKRPQRRR (HIV TAT₄₇₋₅₇; SEQ ID NO: 22), RRWRRWWRRWWRRWRR (W/R; SEQ ID NO: 23), CWK₁₈ (AlkCWK₁₈; SEQ ID NO: 24), K₁₈WCCWK₁₈ (Di-CWK₁₈; SEQ ID NO: 25), WTLNSAGYLLGKINLKALAALAKKIL (Transportan; SEQ ID NO: 26), GLFEALEELWEAK (DipaLytic; SEQ ID NO: 27), K₁₆GGCRGDMFGCAK₁₆RGD (K₁₆RGD; SEQ ID NO: 28), K₁₆GGCMFGCGG (P1; SEQ ID NO: 29), K₁₆ICRRARGDNPDDRCT (P2; SEQ ID NO: 30), KKWKMRRNQFWVKVQRbAK (B) bA (P3; SEQ ID NO: 31), VAYISRGGVSTYYSDTVKGRFTRQKYNKRA (P3a; SEQ ID NO: 32), IGRIDPANGKTKYAPKFQDKATRSNYYGNSPS (P9.3; SEQ ID NO: 33), KETWWETWWTEWSQPKKKRKV (Pep-1; SEQ ID NO: 34), PLAEIDGIELTY (Plae; SEQ ID NO: 35), K₁₆GGPLAEIDGIELGA (Kplae; SEQ ID NO: 36), K₁₆GGPLAEIDGIELCA (cKplae; SEQ ID NO: 37), GALFLGFLGGAAGSTMGAWSQPKSKRKV (MGP; SEQ ID NO: 38), WEAK(LAKA)₂-LAKH(LAKA)₂LKAC (HA2; SEQ ID NO: 39), (LARL)₆NHCH₃ (LARL4₆; SEQ ID NO: 40), KLLKLLLKLWLLKLLL (Hel-11-7; SEQ ID NO: 41), (KKKK)₂GGC (KK; SEQ ID NO: 42), (KWKK)₂GCC (KWK; SEQ ID NO: 43), (RWRR)₂GGC (RWR; SEQ ID NO: 44), PKKKRKV (SV40 NLS7; SEQ ID NO: 45), PEVKKKRKPEYP (NLS12; SEQ ID NO: 46), TPPKKKRKVEDP (NLS12a; SEQ ID NO: 47), GGGGPKKKRKVGG (SV40 NLS13; SEQ ID NO: 48), GGGFSTSLRARKA (AV NLS13; SEQ ID NO: 49), CKKKKKKSEDEYPYVPN (AV RME NLS17; SEQ ID NO: 50), CKKKKKKKSEDEYPYVPNFSTSLRARKA (AV FP NLS28; SEQ ID NO: 51), LVRKKRKTEEESPLKDKDAKKSKQE (SV40 N1 NLS24; SEQ ID NO: 52), and K₉K₂K₄K₈GGK₅ (Loligomer; SEQ ID NO: 53); HSV-1 tegument protein VP22; HSV-1 tegument protein VP22r fused with nuclear export signal (NES); mutant B-subunit of Escherichia coli enterotoxin EtxB (H57S); detoxified exotoxin A (ETA); the protein transduction domain of the HIV-1 Tat protein, GRKKRRQRRRPPQ (SEQ ID NO: 54); the Drosophila melanogaster Antennapedia domain Antp (amino acids 43-58), RQIKIWFQNRRMKWKK (SEQ ID NO: 55); Buforin II, TRSSRAGLQFPVGRVHRLLRK (SEQ ID NO: 56); hClock-(amino acids 35-47) (human Clock protein DNA-binding peptide), KRVSRNKSEKKRR (SEQ ID NO: 57); MAP (model amphipathic peptide), KLALKLALKALKAALKLA (SEQ ID NO: 58); K-FGF, AAVALLPAVLLALLAP (SEQ ID NO: 59); Ku70-derived peptide, comprising a peptide selected from the group comprising VPMLKE (SEQ ID NO: 60), VPMLK (SEQ ID NO: 61), PMLKE (SEQ ID NO: 62) or PMLK (SEQ ID NO: 63); Prion, Mouse Prpe (amino acids 1-28), MAN LGYWLLALFVTMWTDVGLCKKRPKP (SEQ ID NO: 64); pVEC, LLIILRRRIRKQAHAHSK (SEQ ID NO: 65); Pep-I, KETWWETWWTEWSQPKKKRKV (SEQ ID NO: 66); SynBI, RGGRLSYSRRRFSTSTGR (SEQ ID NO: 67); Transportan, GWTLNSAGYLLGKINLKALAALAKKIL (SEQ ID NO: 68); Transportan-10, AGYLLGKINLKALAALAKKIL (SEQ ID NO: 69); CADY, Ac-GLWRALWRLLRSLWRLLWRA-cysteamide (SEQ ID NO: 70); Pep-7, SDLWEMMMVSLACQY (SEQ ID NO: 71); HN-1, TSPLNIHNGQKL (SEQ ID NO: 72); VT5, DPKGDPKGVTVTVTVTVTGKGDPKPD (SEQ ID NO: 73); or pISL, RVIRVWFQNKRCKDKK (SEQ ID NO: 74).

In preferred embodiments, the membrane permeating moiety is a lipid moiety, such as a C₁₀-C₂₀ fatty acyl group, especially stearoyl (octadecanoyl; C₁₈), palmitoyl (hexadecanoyl; C₁₆) or myristoyl (tetradecanoyl; C₁₄); most especially myristoyl. In preferred embodiments, the membrane permeating moiety is coupled to the N- or C-terminal amino acid residue or through the amino group of a lysine side-chain of the proteinaceous molecule or other suitably modified side-chain, especially the N-terminal amino acid residue of the proteinaceous molecule or through the amino group of a lysine side-chain. In particular embodiments, the membrane permeating moiety is coupled through the amino group of the N-terminal amino acid residue.

Accordingly, in another aspect of the present invention, there is provided an isolated or purified proteinaceous molecule represented by Formula II:

M-P (II)

wherein:
M is a membrane permeating moiety; and
P is an isolated or purified proteinaceous molecule represented by Formula I.

In some embodiments, M is coupled at any point on the proteinaceous molecule; especially to the N- or C-terminal amino acid residue or through the amino group of a lysine side-chain of the proteinaceous molecule or other suitably modified side-chain, more especially the N-terminal amino acid residue of the proteinaceous molecule or through the amino group of a lysine side-chain; most especially through the amino group of the N-terminal amino acid residue. Suitable membrane permeating moieties and embodiments of the proteinaceous molecule represented by Formula I are as described herein.

In some embodiments, the proteinaceous molecules of the present invention are cyclic molecules. Without wishing to be bound by theory, cyclization of peptides is thought to decrease the susceptibility of the peptides to degradation. In particular embodiments, the proteinaceous molecules are cyclized using N-to-C cyclization (head to tail cyclization), preferably through an amide bond. Such proteinaceous molecules do not possess N- or C-terminal amino acid residues. In particular embodiments, the proteinaceous molecules have an amide-cyclized peptide backbone. In other embodiments, the peptides are cyclized using side-chain to side-chain cyclization, preferably through a disulfide bond a diselenide bond, a seleno-sulfur bond, a thioether bond such as a lanthionine bond, a selenoether bond, a triazole bond, a lactam bond or a dimethylene bond; especially through a disulfide bond.

In some embodiments, the N- and C-termini are linked using a linking moiety. The linking moiety may be a peptide linker such that cyclization produces an amide-cyclized peptide backbone. Variation within the peptide sequence of the linking moiety is possible, such that the linking moiety may be modified to alter the physicochemical properties of the proteinaceous molecules and potentially reduce side effects of the proteinaceous molecules of the invention or otherwise improve the therapeutic use of the molecules, for example, by improving stability. The linking moiety will be of suitable length to span the distance between the N- and C-termini of the proteinaceous molecule without substantially altering the structural conformation of the proteinaceous molecule, for example, a peptidic linking moiety may be between 2 and 10 amino acid residues in length. In some embodiments, longer or shorter peptidic linking moieties may be required.

The proteinaceous molecules of the present invention may be in the form of salts or prodrugs. The salts of the proteinaceous molecules of the present invention are preferably pharmaceutically acceptable, but it will be appreciated that non-pharmaceutically acceptable salts also fall within the scope of the present invention.

The proteinaceous molecules of the present invention may be in crystalline form and/or in the form of solvates, for example, hydrates. Solvation may be performed using methods known in the art.

The peptides of the present invention may be prepared using recombinant DNA techniques or by chemical synthesis.

In some embodiments, the proteinaceous molecules of the present invention are prepared using recombinant DNA techniques. For example, the proteinaceous molecules of the invention may be prepared by a procedure including the steps of: (a) preparing a construct comprising a polynucleotide sequence that encodes the proteinaceous molecule of the invention and that is operably linked to a regulatory element; (b) introducing the construct into a host cell; (c) culturing the host cell to express the polynucleotide sequence to thereby produce the encoded proteinaceous molecule of the invention; and (d) isolating the proteinaceous molecule of the invention from the host cell. The proteinaceous molecules of the present invention may be prepared recombinantly using standard protocols, for example, as described in Klint, et al. (2013) PLOS One, 8(5): e63865; Sambrook, et al. (1989) Molecular Cloning: A Laboratory Manual (Cold Spring Harbour Press), in particular Sections 16 and 17; Ausubel, et al. (1998) Current Protocols in Molecular Biology (John Wiley and Sons, Inc.), in particular Chapters 10 and 16; Coligan, et al. (1997) Current Protocols in Protein Science (John Wiley and Sons, Inc.), in particular Chapters 1, 5 and 6; and U.S. Pat. No. 5,976,567, the entire contents of which are hereby incorporated by reference.

Thus, the present invention also contemplates nucleic acid molecules which encode a proteinaceous molecule of the invention. Thus, in a further aspect of the present invention, there is provided an isolated nucleic acid molecule comprising a polynucleotide sequence that encodes the proteinaceous molecule of the invention or is complementary to a polynucleotide sequence that encodes a proteinaceous molecule of the invention, such as the proteinaceous molecule of Formula I, any one of SEQ ID NO: 1-21, especially any one of SEQ ID NO: 1-18 or variant proteinaceous molecule as described herein.

The isolated nucleic acid molecules of the present invention may be DNA or RNA. When the nucleic acid molecule is in DNA form, it may be genomic DNA or cDNA. RNA forms of the nucleic acid molecules of the present invention are generally mRNA.

Although the nucleic acid molecules are typically isolated, in some embodiments, the nucleic acid molecules may be integrated into or ligated to or otherwise fused or associated with other genetic molecules, such as an expression vector. Generally, an expression vector includes transcriptional and translational regulatory nucleic acid operably linked to the polynucleotide sequence. Accordingly, in another aspect of the invention, there is provided an expression vector comprising a polynucleotide sequence that encodes a proteinaceous molecule of the invention, such as the proteinaceous molecule of Formula I, any one of SEQ ID NO: 1-21, especially any one of SEQ ID NO: 1-18 or variant proteinaceous molecule as described herein.

Typical vectors contain transcription and translation terminators, transcription and translation initiation sequences and promoters useful for regulation of the expression of the nucleic acid. The vectors optionally comprise generic expression cassettes containing at least one independent terminator sequence, sequences permitting replication of the cassette in eukaryotes, prokaryotes or both, (e.g. shuttle vectors) and selection markers for both prokaryotic and eukaryotic systems. Vectors may be suitable for replication and integration in prokaryotes, eukaryotes, or both. See, Giliman and Smith (1979), Gene, 8: 81-97; Roberts et al. (1987) Nature, 328: 731-734; Berger and Kimmel, Guide to Molecular Cloning Techniques, Methods in Enzymology, volume 152, Academic Press, Inc., San Diego, Calif. (Berger); Sambrook et al. (1989), Molecular Cloning—a Laboratory Manual (2nd ed.) Vol. 1-3, Cold Spring Harbor Laboratory, Cold Spring Harbor Press, N.Y.; and Ausubel et al. (1994) Current Protocols in Molecular Biology, eds., Current Protocols, a joint venture between Greene Publishing Associates, Inc. and John Wiley & Sons, Inc. (Supplement), the entire contents of which are incorporated by reference.

Expression vectors containing regulatory elements from eukaryotic viruses such as retroviruses are typically used for expression of nucleic acid sequences in eukaryotic cells. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter or other promoters shown effective for expression in eukaryotic cells.

While a variety of vectors may be used, it should be noted that viral expression vectors are useful for modifying eukaryotic cells because of the high efficiency with which the viral vectors transfect target cells and integrate into the target cell genome. Illustrative expression vectors of this type can be derived from viral DNA sequences including, but not limited to, adenovirus, adeno-associated viruses, herpes-simplex viruses and retroviruses such as B, C, and D retroviruses as well as spumaviruses and modified lentiviruses. Suitable expression vectors for transfection of animal cells are described, for example, by Wu and Ataai (2000) Curr. Opin. Biotechnol., 11(2): 205-208; Vigna and Naldini (2000) J. Gene Med., 2(5): 308-316; Kay et al. (2001) Nat. Med., 7(1): 33-40; Athanasopoulos et al. (2000) Int. J. Mol. Med., 6(4): 363-375; and Walther and Stein (2000) Drugs, 60(2): 249-271, the entire contents of which are incorporated by reference.

The polypeptide or peptide-encoding portion of the expression vector may comprise a naturally-occurring sequence or a variant thereof, which has been engineered using recombinant techniques. In one example of a variant, the codon composition of a polynucleotide encoding a proteinaceous molecule of the invention is modified to permit enhanced expression of the proteinaceous molecule of the invention in a mammalian host using methods that take advantage of codon usage bias, or codon translational efficiency in specific mammalian cell or tissue types as set forth, for example, in International Publications WO 99/02694 and WO 00/42215. Briefly, these latter methods are based on the observation that translational efficiencies of different codons vary between different cells or tissues and that these differences can be exploited, together with codon composition of a gene, to regulate expression of a protein in a particular cell or tissue type. Thus, for the construction of codon-optimized polynucleotides, at least one existing codon of a parent polynucleotide is replaced with a synonymous codon that has a higher translational efficiency in a target cell or tissue than the existing codon it replaces. Although it is preferable to replace all the existing codons of a parent nucleic acid molecule with synonymous codons which have that higher translational efficiency, this is not necessary because increased expression can be accomplished even with partial replacement. Suitably, the replacement step affects 5%, 10%, 15%, 20%, 25%, 30%, more preferably 35%, 40%, 50%, 60%, 70% or more of the existing codons of a parent polynucleotide.

The expression vector is compatible with the cell in which it is introduced such that the proteinaceous molecule of the invention is expressible by the cell. The expression vector is introduced into the cell by any suitable means which will be dependent on the particular choice of expression vector and cell employed. Such means of introduction are well-known to those skilled in the art. For example, introduction can be effected by use of contacting (e.g. in the case of viral vectors), electroporation, transformation, transduction, conjugation or triparental mating, transfection, infection membrane fusion with cationic lipids, high-velocity bombardment with DNA-coated microprojectiles, incubation with calcium phosphate-DNA precipitate, direct microinjection into single cells, and the like. Other methods also are available and are known to those skilled in the art. Alternatively, the vectors are introduced by means of cationic lipids, e.g., liposomes. Such liposomes are commercially available (e.g. Lipofectin®, Lipofectamine™, and the like, supplied by Life Technologies, Gibco BRL, Gaithersburg, Md.).

In some embodiments, the proteinaceous molecules of the invention may be produced inside a cell by introduction of one or more expression constructs, such as an expression vector, that comprise a polynucleotide sequence that encodes a proteinaceous molecule of the invention.

The invention contemplates recombinantly producing the proteinaceous molecule of the invention inside a host cell, such as a mammalian cell (e.g. Chinese hamster ovary (CHO) cell, mouse myeloma (NSO) cell, baby hamster kidney (BHK) cell or human embryonic kidney (HEK293) cell), yeast cell (e.g. Pichia pastoris cell, Saccharomyces cerevisiae cell, Schizosaccharomyces pombe cell, Hansenula polymorpha cell, Kluyveromyces lactis cell, Yarrowia lipolytica cell or Arxula adeninivorans cell), or bacterial cell (e.g. Escherichia coli cell, Corynebacterium glutamicum or Pseudomonas fluorescens cell).

For therapeutic applications, the invention also contemplates producing the proteinaceous molecules of the invention in vivo inside a cell of a subject, for example a PD-1, PD-L1 and/or PD-L2 overexpressing cell, such as a vertebrate cell, particularly a mammalian or avian cell, especially a mammalian cell.

In some embodiments, the proteinaceous molecules of the present invention are prepared using standard peptide synthesis methods, such as solution synthesis or solid phase synthesis. The chemical synthesis of the proteinaceous molecules of the invention may be performed manually or using an automated synthesizer. For example, the linear peptides may be synthesized using solid phase peptide synthesis using either Boc or Fmoc chemistry, as described in Merrifield (1963) J Am Chem Soc, 85(14): 2149-2154; Schnolzer, et al. (1992) Int J Pept Protein Res, 40: 180-193 and Cardoso, et al. (2015) Mol Pharmacol, 88(2): 291-303, the entire contents of which are incorporated by reference. Following deprotection and cleavage from the solid support, the linear peptides are purified using suitable methods, such as preparative chromatography.

In other embodiments, the proteinaceous molecules of the invention may be cyclized. Cyclization may be performed using several techniques, as described in, for example, Davies (2003) J Pept Sci, 9: 471-501, the entire contents of which are incorporated by reference. In particular embodiments, the linear peptide is synthesized using solid phase peptide synthesis involving Boc-chemistry, starting with a cysteine residue at the N-terminus and ending with a thioester at the C-terminus. Following deprotection and cleavage from the resin, the peptide is cyclized via a thiolactone intermediate, which subsequently rearranges to an amine-cyclized peptide.

### 4. Pharmaceutical Compositions

In accordance with the present invention, the proteinaceous molecules are useful in compositions and methods for the treatment or prevention of a condition involving the nuclear localization of a nuclear localizable polypeptide, such as PD-1, PD-L1 and/or PD-L2, for example a cancer.

Thus, in some embodiments, the proteinaceous molecule of the present invention may be in the form of a pharmaceutical composition, wherein the pharmaceutical composition comprises a proteinaceous molecule of the invention and a pharmaceutically acceptable carrier or diluent.

The proteinaceous molecules of the invention may be formulated into the pharmaceutical compositions as neutral or salt forms.

As will be appreciated by those skilled in the art, the choice of pharmaceutically acceptable carrier or diluent will be dependent on the route of administration and on the nature of the condition and the subject to be treated. The particular carrier or delivery system and route of administration may be readily determined by a person skilled in the art. The carrier or delivery system and route of administration should be carefully selected to ensure that the activity of the proteinaceous molecule is not depleted during preparation of the formulation and the proteinaceous molecule is able to reach the site of action intact. The pharmaceutical compositions of the invention may be administered through a variety of routes including, but not limited to, oral, rectal, topical, intranasal, intraocular, transmucosal, intestinal, enteral, intramuscular, subcutaneous, intramedullary, intrathecal, intraventricular, intracerebral, intravaginal, intravesical, intravenous or intraperitoneal administration.

The pharmaceutical forms suitable for injectable use include sterile injectable solutions or dispersions and sterile powders for the preparation of sterile injectable solutions. Such forms should be stable under the conditions of manufacture and storage and may be preserved against reduction, oxidation and microbial contamination.

A person skilled in the art will readily be able to determine appropriate formulations for the proteinaceous molecules of the invention using conventional approaches. Techniques for formulation and administration may be found in, for example, Remington (1980) Remington's Pharmaceutical Sciences, Mack Publishing Co., Easton, Pa., latest edition; and Niazi (2009) Handbook of Pharmaceutical Manufacturing Formulations, Informa Healthcare, New York, second edition, the entire contents of which are incorporated by reference.

Identification of preferred pH ranges and suitable excipients, such as antioxidants, is routine in the art, for example, as described in Katdare and Chaubel (2006) Excipient Development for Pharmaceutical, Biotechnology and Drug Delivery Systems (CRC Press). Buffer systems are routinely used to provide pH values of a desired range and may include, but are not limited to, carboxylic acid buffers, such as acetate, citrate, lactate, tartrate and succinate; glycine; histidine; phosphate; tris(hydroxymethyl)aminomethane (Tris); arginine; sodium hydroxide; glutamate; and carbonate buffers. Suitable antioxidants may include, but are not limited to, phenolic compounds such as butylated hydroxytoluene (BHT) and butylated hydroxyanisole; vitamin E; ascorbic acid; reducing agents such as methionine or sulfite; metal chelators such as ethylene diamine tetraacetic acid (EDTA); cysteine hydrochloride; sodium bisulfite; sodium metabisulfite; sodium sulfite; ascorbyl palmitate; lecithin; propyl gallate; and alpha-tocopherol.

For injection, the proteinaceous molecules of the invention may be formulated in aqueous solutions, suitably in physiologically compatible buffers such as Hanks' solution, Ringer's solution or physiological saline buffer. For transmucosal administration, penetrants appropriate to the barrier to be permeated are used in the formulation. Such penetrants are generally known in the art.

The compositions of the present invention may be formulated for administration in the form of liquids, containing acceptable diluents (such as saline and sterile water), or may be in the form of lotions, creams or gels containing acceptable diluents or carriers to impart the desired texture, consistency, viscosity and appearance. Acceptable diluents and carriers are familiar to those skilled in the art and include, but are not restricted to, ethoxylated and nonethoxylated surfactants, fatty alcohols, fatty acids, hydrocarbon oils (such as palm oil, coconut oil, and mineral oil), cocoa butter waxes, silicon oils, pH balancers, cellulose derivatives, emulsifying agents such as non-ionic organic and inorganic bases, preserving agents, wax esters, steroid alcohols, triglyceride esters, phospholipids such as lecithin and cephalin, polyhydric alcohol esters, fatty alcohol esters, hydrophilic lanolin derivatives and hydrophilic beeswax derivatives.

Alternatively, the proteinaceous molecules of the present invention can be formulated readily using pharmaceutically acceptable carriers well known in the art into dosages suitable for oral administration, which is also contemplated for the practice of the present invention. Such carriers enable the bioactive agents of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatin, talc, calcium sulfate, vegetable oils, synthetic oils, polyols, alginic acid, phosphate buffered solutions, emulsifiers, isotonic saline and pyrogen-free water.

Pharmaceutical formulations for parenteral administration include aqueous solutions of the proteinaceous molecules of the invention in water-soluble form. Additionally, suspensions of the proteinaceous molecules of the invention may be prepared as appropriate oily injection suspensions. Suitable lipophilic solvents or vehicles include fatty oils such as sesame oil, or synthetic fatty acid esters, such as ethyl oleate or triglycerides. Aqueous injection suspensions may contain substances that increase the viscosity of the suspension, such as sodium carboxymethyl cellulose, sorbitol or dextran. Optionally, the suspension may also contain suitable stabilizers or agents that increase the solubility of the compounds to allow for the preparation of highly concentrated solutions.

Sterile solutions may be prepared by combining the active compounds in the required amount in the appropriate solvent with other excipients as described above as required, followed by sterilization, such as filtration. Generally, dispersions are prepared by incorporating the various sterilized active compounds into a sterile vehicle which contains the basic dispersion medium and the required excipients as described above. Sterile dry powders may be prepared by vacuum- or freeze-drying a sterile solution comprising the active compounds and other required excipients as described above.

Pharmaceutical preparations for oral use can be obtained by combining the proteinaceous molecules of the invention with solid excipients and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, e.g. by means of conventional mixing, dissolving, granulating, dragee-making, levigating, emulsifying, encapsulating, entrapping or lyophilizing processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of particle doses.

Pharmaceuticals which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The proteinaceous molecules of the invention may be incorporated into modified-release preparations and formulations, for example, polymeric microsphere formulations, and oil- or gel-based formulations.

In particular embodiments, the proteinaceous molecule of the invention may be administered in a local rather than systemic manner, such as by injection of the proteinaceous molecule directly into a tissue, which is preferably subcutaneous or omental tissue, often in a depot or sustained release formulation.

Furthermore, the proteinaceous molecule of the invention may be administered in a targeted drug delivery system, such as in a particle which is suitably targeted to and taken up selectively by a cell or tissue. In some embodiments, the proteinaceous molecule of the invention is contained in or otherwise associated with a vehicle selected from liposomes, micelles, dendrimers, biodegradable particles, artificial DNA nanostructure, lipid-based nanoparticles and carbon or gold nanoparticles. In illustrative examples of this type, the vehicle is selected from poly(lactic acid) (PLA), poly(glycolic acid) (PGA), poly(lactic-co-glycolic acid) (PLGA), poly(ethylene glycol) (PEG), PLA-PEG copolymers and combinations thereof.

In cases of local administration or selective uptake, the effective local concentration of the agent may not be related to plasma concentration.

It is advantageous to formulate the compositions in dosage unit form for ease of administration and uniformity of dosage. The determination of the novel dosage unit forms of the present invention is dictated by and directly dependent on the unique characteristics of the active material, the particular therapeutic effect to be achieved and the limitations inherent in the art of compounding active materials for the treatment of disease in living subjects having a diseased condition in which bodily health is impaired as herein disclosed in detail.

While the proteinaceous molecule of the invention may be the sole active ingredient administered to the subject, the administration of other cancer therapies concurrently with said proteinaceous molecule is within the scope of the invention. For example, the proteinaceous molecule of Formula I, any one of SEQ ID NO: 1-21, especially any one of SEQ ID NO: 1-18 or variant described herein may be administered concurrently with one or more cancer therapies, non-limiting examples of which include radiotherapy, surgery, chemotherapy, hormone ablation therapy, pro-apoptosis therapy and immunotherapy. The proteinaceous molecule of the invention may be therapeutically used before treatment with the cancer therapy, may be therapeutically used after the cancer therapy or may be therapeutically used together with the cancer therapy.

Suitable radiotherapies include radiation and waves that induce DNA damage, for example, y-irradiation, X-rays, UV irradiation, microwaves, electronic emissions and radioisotopes. Typically, therapy may be achieved by irradiating the localized tumor site with the above described forms of radiations. It is most likely that all of these factors cause a broad range of damage to DNA, on the precursors of DNA, on the replication and repair of DNA and on the assembly and maintenance of chromosomes.

The dosage range for X-rays ranges from daily doses of 50-200 roentgens for prolonged periods of time such as 3-4 weeks, to single doses of 2000-6000 roentgens. Dosage ranges for radioisotopes vary widely and depend on the half life of the isotope, the strength and type of radiation emitted and the uptake by the neoplastic cells. Suitable radiotherapies may include, but are not limited to, conformal external beam radiotherapy (50-100 Gray given as fractions over 4-8 weeks), either single shot or fractionated high dose brachytherapy, permanent interstitial brachytherapy and systemic radioisotopes such as Strontium 89. In some embodiments, the radiotherapy may be administered with a radiosensitizing agent. Suitable radiosensitizing agents may include, but are not limited to, efaproxiral, etanidazole, fluosol, misonidazole, nimorazole, temoporfin and tirapazamine.

Suitable chemotherapeutic agents may include, but are not limited to, antiproliferative/antineoplastic drugs and combinations thereof including alkylating agents (for example cisplatin, carboplatin, cyclophosphamide, nitrogen mustard, melphalan, chlorambucil, busulphan and nitrosoureas), antimetabolites (for example antifolates such as fluoropyridines like 5-fluorouracil and tegafur, raltitrexed, methotrexate, cytosine arabinoside and hydroxyurea), anti-tumor antibiotics (for example anthracyclines like adriamycin, bleomycin, doxorubicin, daunomycin, epirubicin, idarubicin, mitomycin-C, dactinomycin and mithramycin), antimitotic agents (for example Vinca alkaloids like vincristine, vinblastine, vindesine and vinorelbine and taxoids like paclitaxel and docetaxel), and topoisomerase inhibitors (for example epipodophyllotoxins like etoposide and teniposide, amsacrine, topotecan and camptothecin); cytostatic agents such as antiestrogens (for example tamoxifen, toremifene, raloxifene, droloxifene and idoxifene), estrogen receptor down regulators (for example fulvestrant), antiandrogens (for example bicalutamide, flutamide, nilutamide and cyproterone acetate), UH antagonists or LHRH agonists (for example goserelin, leuprorelin and buserelin), progestogens (for example megestrol acetate), aromatase inhibitors (for example as anastrozole, letrozole, vorozole and exemestane) and inhibitors of 5cc-reductase such as finasteride; agents which inhibit cancer cell invasion (for example metalloproteinase inhibitors like marimastat and inhibitors of urokinase plasminogen activator receptor function); inhibitors of growth factor function, for example such inhibitors include growth factor antibodies, growth factor receptor antibodies (for example the anti-erbb2 antibody trastuzumab [Herceptin™] and the anti-erbbl antibody Cetuximab [C225]), farnesyl transferase inhibitors, MEK inhibitors, tyrosine kinase inhibitors and serine/threonine kinase inhibitors, for example other inhibitors of the epidermal growth factor family (for example other EGFR family tyrosine kinase inhibitors such as N-(3-chloro-4-fluorophenyl)-7-methoxy-6-(3-morpholinopropoxy)quinazolin-4-amine (Gefitinib, AZD1839), N-(3-ethynylphenyl)-6,7-bis(2-methoxyethoxy)quinazolin-4-amine (Erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family; anti-angiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin avβ3 function and angiostatin); cyclin-dependent kinase inhibitors such as palbociclib, abemaciclib, ribociclib and alvociclib; vascular damaging agents such as Combretastatin A4 and compounds disclosed in International Patent Applications WO 99/02166, WO00/40529, WO 00/41669, WO01/92224, WO02/04434 and WO02/08213; antisense therapies, for example those which are directed to the targets listed above, such as ISIS 2503, an anti-ras antisense; and gene therapy approaches, including for example approaches to replace aberrant genes such as aberrant p53 or aberrant GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy.

Suitable immunotherapy approaches may include, but are not limited to ex vivo and in vivo approaches to increase the immunogenicity of patient tumor cells such as transfection with cytokines including interleukin 2, interleukin 4 or granulocyte-macrophage colony stimulating factor; approaches to decrease T-cell anergy; approaches using transfected immune cells such as cytokine-transfected dendritic cells; approaches using cytokine-transfected tumor cell lines; and approaches using anti-idiotypic antibodies. These approaches generally rely on the use of immune effector cells and molecules to target and destroy cancer cells. The immune effector may be, for example, an antibody specific for some marker on the surface of a malignant cell. The antibody alone may serve as an effector of therapy or it may recruit other cells to actually facilitate cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a malignant cell target. Various effector cells include cytotoxic T cells and NK cells.

In some embodiments, the immune effector is a molecule targeting PD-L1, including, but not limited to, an anti-PD-L1 antibody, non-limiting examples of which include atezolizumab, avelumab, durvalumab, BMS-936559, BMS-935559, the antibodies described in WO 2013/173223 A1, WO 2013/079174 A1, WO 2010/077634 A1, WO 2011/066389 A1, CN 101104640 A, WO 2010/036959 A2, WO 2007/005874 A2, WO 2004/004771 A1, WO 2006/133396 A2, WO 2013/181634 A2, WO 2012/145493 A1, clone EH12, and clone 29E.2A3; CA-170; CA-327; BMS-202 (N-[2-[[[2-methoxy-6-[(2-methyl[1,1′-biphenyl]-3-yl)methoxy]-3-pyridinyl]methyl]amino]ethyl]-acetamide); BMS-8 (1-[[3-bromo-4-[(2-methyl[1,1′-biphenyl]-3-yl)methoxy]phenyl]methyl]-2-piperidinecarboxylic acid); the peptides described in Chang et al. (2015) Angew Chem Int Ed Engl, 54(40): 11760-11764, especially (D) PPA-1; AUNP-12; and the peptides described in WO 2014/151634 A1, the entire contents of which are incorporated by reference.

In some embodiments, the immune effector is a molecule targeting PD-1 including, but not limited to, an anti-PD-1 antibody, non-limiting examples of which include nivolumab, pembrolizumab, BGB-A317, the antibodies described in WO 2016/106159 A1, WO 2009/114335 A2, WO 2004/004771 A1, WO 2013/173223 A1, WO 2015/112900 A1, WO 2008/156712 A1, WO 2011/159877 A2, WO 2010/036959 A2, WO 2010/089411 A2, WO 2006/133396 A2, WO 2012/145493 A1, WO 2002/078731 A1, anti-mouse PD-1 antibody clone J43, anti-mouse antibody clone RMP1-14, ANB011 (TSR-042), AMP-514 (MEDI0680), WO 2006/121168 A1, WO 2001/014557 A1, WO 2011/110604 A1, WO 2011/110621 A1, WO 2004/072286 A1, WO 2004/056875 A1, WO 2010/036959 A2, WO 2010/029434 A1, and WO 2013/022091 A1; AMP-224; the compounds described in WO 2011/082400 A2; the molecules and antibodies described in U.S. Pat. No. 6,808,710 B1; the molecules and antibodies described in WO 2013/019906 A1; the molecules described in WO 2003/011911 A1; and the compounds described in WO 2013/132317 A1, the entire contents of which are incorporated by reference.

In some embodiments, the immune effector is a molecule targeting PD-L2 including, but not limited to, an anti-PD-L2 antibody, non-limiting examples of which include the antibodies described in WO 2010/036959 A2, the entire content of which is incorporated by reference; and rHigM12B7.

In some embodiments, the immune effector is a molecule targeting CTLA-4 including, but not limited to, an anti-CTLA-4 antibody such as ipilimumab, tremelimumab, the antibodies described in WO 00/37504 A2, WO 01/14424 A2, US 2003/0086930 A1; and the compounds described in WO 2006/056464 A2, the entire contents of which are incorporated by reference.

Examples of other cancer therapies include phytotherapy, cryotherapy, toxin therapy or pro-apoptosis therapy. A person skilled in the art would appreciate that this list is not exhaustive of the types of treatment modalities available for cancer and other hyperplastic lesions.

It is well known that chemotherapy and radiation therapy target rapidly dividing cells and/or disrupt the cell cycle or cell division. These treatments are offered as part of treating several forms of cancer, aiming either at slowing their progression or reversing the symptoms of disease by means of a curative treatment. However, these cancer treatments may lead to an immunocompromised state and ensuing pathogenic infections and, thus, the present invention also extends to combination therapies, which employ a proteinaceous molecule of Formula I, any one of SEQ ID NO: 1-21, especially any one of SEQ ID NO: 1-18 or variant described herein, a cancer therapy and an anti-infective agent that is effective against an infection that develops or that has an increased risk of developing from an immunocompromised condition resulting from the cancer therapy. The anti-infective drug is suitably selected from antimicrobials, which may include, but are not limited to, compounds that kill or inhibit the growth of microorganisms such as viruses, bacteria, yeast, fungi, protozoa, etc. and, thus, include antibiotics, amebicides, antifungals, antiprotozoals, antimalarials, antituberculotics and antivirals. Anti-infective drugs also include within their scope anthelmintics and nematocides. Illustrative antibiotics include quinolones (e.g. amifloxacin, cinoxacin, ciprofloxacin, enoxacin, fleroxacin, flumequine, lomefloxacin, nalidixic acid, norfloxacin, ofloxacin, levofloxacin, lomefloxacin, oxolinic acid, pefloxacin, rosoxacin, temafloxacin, tosufloxacin, sparfloxacin, clinafloxacin, gatifloxacin, moxifloxacin; gemifloxacin; and garenoxacin), tetracyclines, glycylcyclines and oxazolidinones (e.g. chlortetracycline, demeclocycline, doxycycline, lymecycline, methacycline, minocycline, oxytetracycline, tetracycline, tigecycline; linezolide, eperezolid), glycopeptides, aminoglycosides (e.g. amikacin, arbekacin, butirosin, dibekacin, fortimicins, gentamicin, kanamycin, menomycin, netilmicin, ribostamycin, sisomicin, spectinomycin, streptomycin, tobramycin), β-lactams (e.g. imipenem, meropenem, biapenem, cefaclor, cefadroxil, cefamandole, cefatrizine, cefazedone, cefazolin, cefixime, cefmenoxime, cefodizime, cefonicid, cefoperazone, ceforanide, cefotaxime, cefotiam, cefpimizole, cefpiramide, cefpodoxime, cefsulodin, ceftazidime, cefteram, ceftezole, ceftibuten, ceftizoxime, ceftriaxone, cefuroxime, cefuzonam, cephacetrile, cephalexin, cephaloglycin, cephaloridine, cephalothin, cephapirin, cephradine, cefinetazole, cefoxitin, cefotetan, azthreonam, carumonam, flomoxef, moxalactam, amdinocillin, amoxicillin, ampicillin, azlocillin, carbenicillin, benzylpenicillin, carfecillin, cloxacillin, dicloxacillin, methicillin, mezlocillin, nafcillin, oxacillin, penicillin G, piperacillin, sulbenicillin, temocillin, ticarcillin, cefditoren, SC004, KY-020, cefdinir, ceftibuten, FK-312, S-1090, CP-0467, BK-218, FK-037, DQ-2556, FK-518, cefozopran, ME1228, KP-736, CP-6232, Ro 09-1227, OPC-20000, LY206763), rifamycins, macrolides (e.g. azithromycin, clarithromycin, erythromycin, oleandomycin, rokitamycin, rosaramicin, roxithromycin, troleandomycin), ketolides (e.g. telithromycin, cethromycin), coumermycins, lincosamides (e.g. clindamycin, lincomycin) and chloramphenicol.

Illustrative antivirals include abacavir sulfate, acyclovir sodium, amantadine hydrochloride, amprenavir, cidofovir, delavirdine mesylate, didanosine, efavirenz, famciclovir, fomivirsen sodium, foscarnet sodium, ganciclovir, indinavir sulfate, lamivudine, lamivudine/zidovudine, nelfinavir mesylate, nevirapine, oseltamivir phosphate, ribavirin, rimantadine hydrochloride, ritonavir, saquinavir, saquinavir mesylate, stavudine, valacyclovir hydrochloride, zalcitabine, zanamivir and zidovudine.

Suitable amebicides or antiprotozoals include, but are not limited to, atovaquone, chloroquine hydrochloride, chloroquine phosphate, metronidazole, metronidazole hydrochloride and pentamidine isethionate. Anthelmintics can be at least one selected from mebendazole, pyrantel pamoate, albendazole, ivermectin and thiabendazole. Illustrative antifungals can be selected from amphotericin B, amphotericin B cholesteryl sulfate complex, amphotericin B lipid complex, amphotericin B liposomal, fluconazole, flucytosine, griseofulvin microsize, griseofulvin ultramicrosize, itraconazole, ketoconazole, nystatin and terbinafine hydrochloride. Suitable antimalarials include, but are not limited to, chloroquine hydrochloride, chloroquine phosphate, doxycycline, hydroxychloroquine sulfate, mefloquine hydrochloride, primaquine phosphate, pyrimethamine and pyrimethamine with sulfadoxine. Antituberculotics include but are not restricted to clofazimine, cycloserine, dapsone, ethambutol hydrochloride, isoniazid, pyrazinamide, rifabutin, rifampin, rifapentine and streptomycin sulfate.

As previously described, the proteinaceous molecule may be compounded for convenient and effective administration in effective amounts with a suitable pharmaceutically acceptable carrier in dosage unit form. In some embodiments, a unit dosage form may comprise the active peptide of the invention in amount in the range of from about 0.25 μg to about 2000 mg. The active peptide of the invention may be present in an amount of from about 0.25 μg to about 2000 mg/mL of carrier. In embodiments where the pharmaceutical composition comprises one or more additional active ingredients, the dosages are determined by reference to the usual dose and manner of administration of the said ingredients.

### 5. Methods

The present inventors have determined that proteinaceous molecules comprising an amino acid sequence corresponding to an acetylation site inhibit or reduce nuclear localization of a nuclear localizable polypeptide wherein acetylation of an acetylation site of the nuclear localizable polypeptide increases its nuclear localization in a cell. In particular, the present inventors have found that a proteinaceous molecule corresponding to an acetylation site in PD-L1, especially a proteinaceous molecule corresponding to residues 255 to 271 of PD-L1, reduces or inhibits nuclear localization of PD-1, PD-L1 and PD-L2. The present inventors have conceived that the proteinaceous molecules of the invention may be useful in methods for altering at least one of formation, proliferation, maintenance, EMT, MET or viability of a PD-1, PD-L1 and/or PD-L2 overexpressing cell and are useful for the treatment or prevention of a condition involving PD-1, PD-L1 and/or PD-L2 nuclear localization in a subject, such as a cancer.

Without wishing to be bound by theory, the inventors have determined that acetylation of particular nuclear localizable polypeptides, such as an immune checkpoint protein including PD-1, PD-L1 and/or PD-L2, increase nuclear localization of the polypeptide and, thus, it is proposed that inhibition of acetylation of the nuclear localizable polypeptide will also inhibit or reduce nuclear localization of the polypeptide. Furthermore, it is proposed that a proteinaceous molecule which corresponds to an acetylation site will competitively inhibit acetylation of the nuclear localizable polypeptide and, thus, decrease nuclear localization of the polypeptide.

Accordingly, in another aspect of the invention, there is provided a method of inhibiting or reducing the nuclear localization of a nuclear localizable polypeptide wherein acetylation of an acetylation site of the nuclear localizable polypeptide increases its nuclear localization in a cell, the method comprising contacting the cell with a proteinaceous molecule comprising, consisting or consisting essentially of an amino acid sequence corresponding to an acetylation site. The present invention also provides a use of a proteinaceous molecule comprising, consisting or consisting essentially of an amino acid corresponding to an acetylation site for inhibiting or reducing the nuclear localization of a nuclear localizable polypeptide wherein acetylation of an acetylation site of the nuclear localizable polypeptide increases its nuclear localization in a cell; and a proteinaceous molecule comprising, consisting or consisting essentially of an amino acid corresponding to an acetylation site for use in inhibiting or reducing the nuclear localization of a nuclear localizable polypeptide wherein acetylation of an acetylation site of the nuclear localizable polypeptide increases its nuclear localization in a cell.

In some embodiments, the nuclear localizable polypeptide is an immune checkpoint protein, especially PD-L1, PD-L2 and/or PD-1. In preferred embodiments, the nuclear localizable polypeptide is PD-L1.

The amino acid sequence corresponding to an acetylation site may be any amino acid sequence which corresponds to an amino acid sequence that may be acetylated, for example, by an acetyltransferase; especially a histone acetyltransferase, including, but not limited to, GCN5, Hat1, ATF-2, Tip60, MOZ, MORF, HBO1, p300, CBP, SRC-1, ACTR, TIF-2, SRC-3, TAF1, TFIIIC and/or CLOCK; especially p300.

In particular embodiments, the amino acid sequence of the proteinaceous molecule corresponds to a lysine acetylation site (i.e. an acetylation site wherein a lysine residue is acetylated); especially a PD-L1 lysine acetylation site; most especially residues 255 to 271 of PD-L1.

The amino acid sequence of PD-L1 (Uniprot No. Q9NZQ7) is presented in SEQ ID NO: 75. The amino acid sequence corresponding to residues 255 to 271 of PD-L1 comprises a potential acetylation site, wherein the E-amino group on lysine 263 is acetylated. Residues 255 to 271 are underlined in the sequence below.

[SEQ ID NO: 75]
MRIFAVFIFM TYWHLLNAFT VTVPKDLYVV EYGSNMTIEC
KFPVEKQLDL AALIVYWEME DKNIIQFVHG EEDLKVQHSS
YRQRARLLKD QLSLGNAALQ ITDVKLQDAG VYRCMISYGG
ADYKRITVKV NAPYNKINQR ILVVDPVTSE HELTCQAEGY
PKAEVIWTSS DHQVLSGKTT TTNSKREEKL FNVTSTLRIN
TTTNEIFYCT FRRLDPEENH TAELVIPELP LAHPPNERTH
LVILGAILLC LGVALTFIFR LRKGRMMDVK KCGIQDTNSK
KQSDTHLEET.

In some embodiments, the proteinaceous molecule is an isolated or purified proteinaceous molecule represented by Formula I; particularly the proteinaceous molecule of any one of SEQ ID NO: 1-21, especially any one of SEQ ID NO: 1-18, or variant proteinaceous molecule described herein.

In preferred embodiments, the proteinaceous molecule is other than a proteinaceous molecule corresponding to an acetylation site on histone 3, especially a site when lysine 4 is acetylated (H3K4) such as residues 1-21 of histone 3, such as the proteinaceous molecules described in Kumarasinghe and Woster (2014) ACS Med. Chem. Lett., 5:29-33; Culhane, et al. (2010) J. Am. Chem. Soc., 132(9):3164-3176; Culhane, et al. (2006) J. Am. Chem. Soc., 128(14):4536-4537; Szewczuk, et al. (2007) Biochemistry, 46(23): 6892-6902; Yang, et al. (2007) Nature Structural and Molecular Biology, 14(6): 535-539; Forneris, et al. (2007) J. Biol. Chem., 282(28): 20070-20074; Forneris, et al. (2005) J. Biol. Chem., 280(50): 41360-41365; and U.S. Pat. No. 9,186,391 B2. In particular embodiments, the proteinaceous molecule is other than a proteinaceous molecule corresponding to the following molecules:

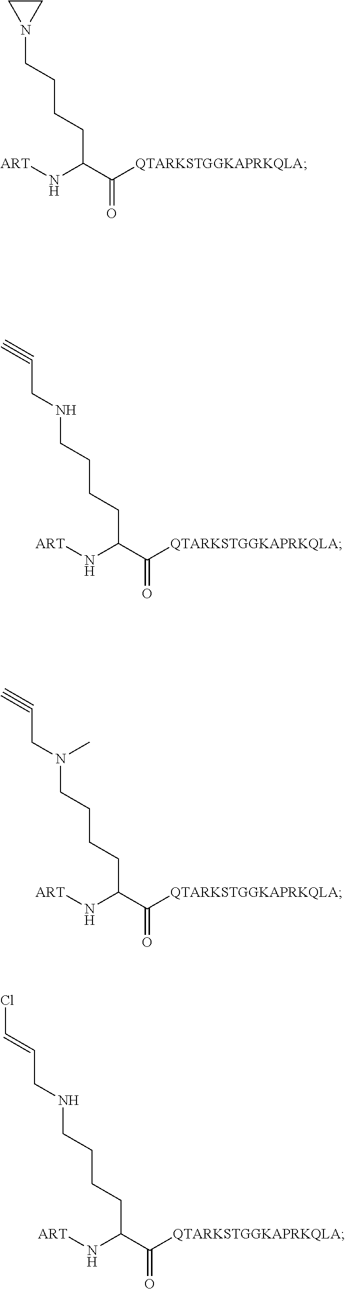

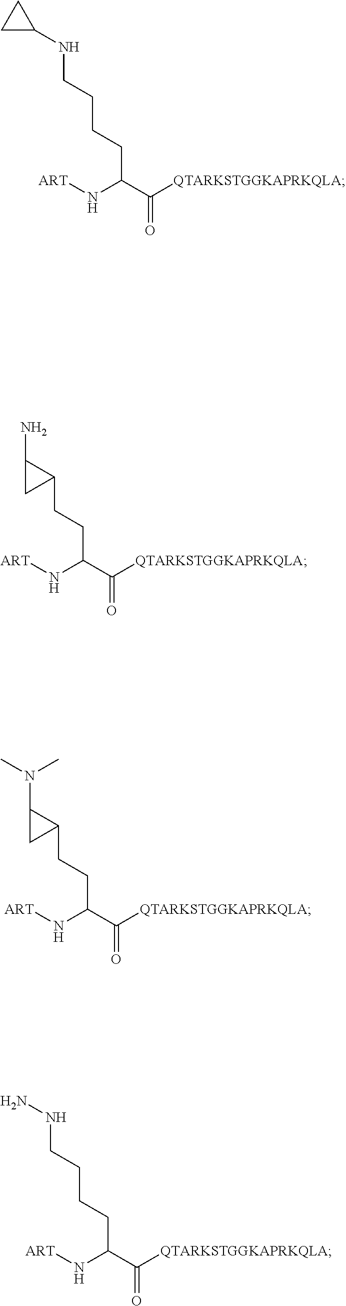

[SEQ ID NO: 76]
ARTKQTARKSTGGKAPRKQLA;
[SEQ ID NO: 77]
ARTMQTARKSTGGKAPRKQLA;
[SEQ ID NO: 78]
AKTMQTARKSTGGEAPRKQLA;
[SEQ ID NO: 79]
ARTMKTARKETGGKAPRKQLA;
[SEQ ID NO: 80]
AKTMQTARKETGGKAPRKQLA;
[SEQ ID NO: 81]
AKTMQTARKSTEGKAPRKQLA;
[SEQ ID NO: 82]
AKTMETARKSTGGKAPRKQLA;
[SEQ ID NO: 83]
ARTMQTARKSTGGEAPRKQLA;

or flavin or biotin conjugates, or salts or prodrugs thereof.

In another aspect of the invention, there is provided a use of the isolated or purified proteinaceous molecule of the invention, particularly the proteinaceous molecule of Formula I, any one of SEQ ID NO: 1-21, especially any one of SEQ ID NO: 1-18, or variant proteinaceous molecule described herein, for therapy or in the manufacture of a medicament for therapy. The invention also provides an isolated or purified proteinaceous molecule of the invention, particularly the proteinaceous molecule of Formula I, any one of SEQ ID NO: 1-21, especially any one of SEQ ID NO: 1-18, or variant proteinaceous molecule described herein, for use in therapy.

The present invention also provides a method of inhibiting or reducing nuclear localization of PD-1, PD-L1 or PD-L2 in a PD-1-, PD-L1- or PD-L2-overexpressing cell, comprising contacting the cell with a proteinaceous molecule comprising, consisting or consisting essentially of an amino acid sequence corresponding to an acetylation site. The present invention also contemplates the use of a proteinaceous molecule comprising, consisting or consisting essentially of an amino acid sequence corresponding to an acetylation site for inhibiting or reducing nuclear localization of PD-1, PD-L1 or PD-L2 in a PD-1-, PD-L1- or PD-L2-overexpressing cell; a proteinaceous molecule comprising, consisting or consisting essentially of an amino acid sequence corresponding to an acetylation site for use in inhibiting or reducing the nuclear localization of PD-1, PD-L1 or PD-L2 in a PD-1-, PD-L1- or PD-L2-overexpressing cell; and in the manufacture of a medicament for such use.

In yet another aspect of the invention, there is provided a method of altering at least one of (i) formation; (ii) proliferation; (iii) maintenance; (iv) EMT; (v) MET; or (vi) viability of a PD-1-, PD-L1- or PD-L2-overexpressing cell, comprising contacting said cell with a formation-, proliferation-, maintenance-, EMT-, MET-or viability-modulating amount of a proteinaceous molecule comprising, consisting or consisting essentially of an amino acid sequence corresponding to an acetylation site. The invention also contemplates a use of a proteinaceous molecule comprising, consisting or consisting essentially of an amino acid sequence corresponding to an acetylation site for altering at least one of (i) formation; (ii) proliferation; (iii) maintenance; (iv) EMT; (v) MET; or (vi) viability of a PD-1-, PD-L1- or PD-L2-overexpressing cell. The invention also extends to a proteinaceous molecule comprising, consisting or consisting essentially of an amino acid sequence corresponding to an acetylation site for use in altering at least one of (i) formation; (ii) proliferation; (iii) maintenance; (iv) EMT; (v) MET; or (vi) viability of a PD-1-, PD-L1- or PD-L2-overexpressing cell; and in the manufacture of a medicament for this use.

In some embodiments of any one of the above aspects, the PD-1-, PD-L1- or PD-L2-overexpressing cell is a cancer stem cell or a non-cancer stem cell tumor cell, especially a cancer stem cell tumor cell.

In some embodiments, the proteinaceous molecule results in a reduction, impairment, abrogation, inhibition or prevention of the (i) formation; (ii) proliferation; (iii) maintenance; (iv) EMT or (vi) viability of a PD-1-, PD-L1- or PD-L2-overexpressing cell; and/or in the enhancement of (v) MET of a PD-1-, PD-L1- or PD-L2-overexpressing cell.

Suitable embodiments of the proteinaceous molecule are as described herein.

Proteinaceous molecules comprising, consisting or consisting essentially of an amino acid sequence corresponding to an acetylation site as described herein, especially the proteinaceous molecules of Formula I, any one of SEQ ID NO: 1-21, especially any one of SEQ ID NO: 1-18 or variant proteinaceous molecule, are useful for the inhibition of nuclear localization of PD-1, PD-L1 and/or PD-L2. Accordingly, the present inventors have conceived that the proteinaceous molecules are useful for treating or preventing a cancer in a subject. Thus, in another aspect, there is provided a method for treating or preventing a cancer in a subject wherein the cancer comprises at least one PD-1-, PD-L1- or PD-L2-overexpressing cell, comprising administering to the subject a proteinaceous molecule comprising, consisting or consisting essentially of an amino acid sequence corresponding to an acetylation site. The present invention also extends to a use of a proteinaceous molecule comprising, consisting or consisting essentially of an amino acid sequence corresponding to an acetylation site for treating or preventing a cancer in a subject wherein the cancer comprises at least one PD-1-, PD-L1- or PD-L2-overexpressing cell; and in the manufacture of a medicament for this purpose. A proteinaceous molecule comprising, consisting or consisting essentially of an amino acid sequence corresponding to an acetylation site for use in treating or preventing a cancer in a subject wherein the cancer comprises at least one PD-1-, PD-L1- or PD-L2-overexpressing cell is also contemplated.

The cancer may be any cancer involving overexpression of PD-1, PD-L1 and/or PD-L2. Suitable cancers may include, but are not limited to breast, prostate, lung, bladder, pancreatic, colon, liver, ovarian, kidney or brain cancer, or melanoma or retinoblastoma; especially breast cancer, lung cancer or melanoma; most especially breast cancer or melanoma; more especially breast cancer.

In some embodiments, the proteinaceous molecules comprising, consisting or consisting essentially of an amino acid sequence corresponding to an acetylation site as described herein are useful for treating, preventing and/or relieving the symptoms of a malignancy, particularly a metastatic cancer. In preferred embodiments, the proteinaceous molecules are used for treating, preventing and/or relieving the symptoms of a metastatic cancer. Suitable types of metastatic cancer include, but are not limited to, metastatic breast, prostate, lung, bladder, pancreatic, colon, liver, ovarian, kidney or brain cancer, or melanoma or retinoblastoma. In some embodiments, the brain cancer is a glioma. In preferred embodiments, the metastatic cancer is metastatic breast cancer, lung cancer or melanoma; especially metastatic breast cancer or melanoma; most especially metastatic breast cancer.

The proteinaceous molecules are useful in methods involving PD-1-, PD-L1- and/or PD-L2-overexpressing cells. In particular embodiments, the PD-1-, PD-L1- and/or PD-L2-overexpressing cell is selected from a breast, prostate, testicular, lung, bladder, pancreatic, colon, melanoma, leukemia, retinoblastoma, liver, ovary, kidney or brain cell; especially a breast, lung or melanoma cell; most especially a breast or melanoma cell; more especially a breast cell. In preferred embodiments, the PD-1-, PD-L1- and/or PD-L2-overexpressing cell is a breast epithelial cell, especially a breast ductal epithelial cell.

In some embodiments, the PD-1-, PD-L1- and/or PD-L2-overexpressing cell is a cancer stem cell or a non-cancer stem cell tumor cell; especially a cancer stem cell tumor cell; most especially a breast cancer stem cell tumor cell. In some embodiments, the cancer stem cell tumor cell expresses CD24 and CD44, particularly CD44^{high}, CD24^{low}.

In some embodiments, the methods further comprise detecting overexpression of a PD-1, PD-L1 and/or PD-L2 gene in a tumor sample obtained from the subject, wherein the tumor sample comprises the cancer stem cell tumor cells and optionally the non-cancer stem cell tumor cells, prior to administering the proteinaceous molecule to the subject.

The proteinaceous molecules comprising, consisting or consisting essentially of an amino acid sequence corresponding to an acetylation site as described herein are suitable for treating an individual who has been diagnosed with a cancer, who is suspected of having a cancer, who is known to be susceptible and who is considered likely to develop a cancer, or who is considered to develop a recurrence of a previously treated cancer. The cancer may be hormone receptor negative. In some embodiments, the cancer is hormone receptor negative and is, thus, resistant to hormone or endocrine therapy. In some embodiments where the cancer is breast cancer, the breast cancer is hormone receptor negative. In some embodiments, the breast cancer is estrogen receptor negative and/or progesterone receptor negative.

There are numerous conditions involving PD-1, PD-L1 and/or PD-L2 overexpression in which the proteinaceous molecules comprising, consisting or consisting essentially of an amino acid sequence corresponding to an acetylation site as described herein may be useful. Accordingly, in another aspect of the invention, there is provided a method of treating or preventing a condition in a subject in respect of which inhibition or reduction of nuclear localization of PD-1, PD-L1 and/or PD-L2 is associated with effective treatment, comprising administering to the subject a proteinaceous molecule comprising, consisting or consisting essentially of an amino acid sequence corresponding to an acetylation site. The invention also provides a use of a proteinaceous molecule comprising, consisting or consisting essentially of an amino acid sequence corresponding to an acetylation site for treating or preventing a condition in a subject in respect of which inhibition or reduction of nuclear localization of PD-1, PD-L1 and/or PD-L2 is associated with effective treatment; a proteinaceous molecule comprising, consisting or consisting essentially of an amino acid sequence corresponding to an acetylation site for use in treating or preventing a condition in a subject in respect of which inhibition or reduction of nuclear localization of PD-1, PD-L1 and/or PD-L2 is associated with effective treatment; and a use of a proteinaceous molecule comprising, consisting or consisting essentially of an amino acid sequence corresponding to an acetylation site in the manufacture of a medicament for this purpose.

Non-limiting examples of conditions involving PD-1, PD-L1 and/or PD-L2 overexpression include cancer, infection, autoimmune disorders and respiratory disorders.

In some embodiments, the infection is a pathogenic infection. The infection may be selected from, but is not limited to, a viral, bacterial, yeast, fungal, helminth or protozoan infection. Viral infections contemplated by the present invention include, but are not restricted to, infections caused by HIV, hepatitis, influenza virus, Japanese encephalitis virus, Epstein-Barr virus, herpes simplex virus, filovirus, human papillomavirus, human T-cell lymphotropic virus, human retrovirus, cytomegalovirus, varicella-zoster virus, poliovirus, measles virus, rubella virus, mumps virus, adenovirus, enterovirus, rhinovirus, ebola virus, west nile virus and respiratory syncytial virus; especially infections caused by HIV, hepatitis, influenza virus, Japanese encephalitis virus, Epstein-Barr virus and respiratory syncytial virus. Bacterial infections include, but are not restricted to, those caused by Neisseria species, Meningococcal species, Haemophilus species, Salmonella species, Streptococcal species, Legionella species, Mycoplasma species, Bacillus species, Staphylococcus species, Chlamydia species, Actinomyces species, Anabaena species, Bacteroides species, Bdellovibrio species, Bordetella species, Borrelia species, Campylobacter species, Caulobacter species, Chlrorbium species, Chromatium species, Chlostridium species, Corynebacterium species, Cytophaga species, Deinococcus species, Escherichia species, Francisella species, Helicobacter species, Haemophilus species, Hyphomicrobium species, Leptospira species, Listeria species, Micrococcus species, Myxococcus species, Nitrobacter species, Oscillatoria species, Prochloron species, Proteus species, Pseudomonas species, Rhodospirillum species, Rickettsia species, Shigella species, Spirillum species, Spirochaeta species, Streptomyces species, Thiobacillus species, Treponema species, Vibrio species, Yersinia species, Nocardia species and Mycobacterium species; especially infections caused by Neisseria species, Meningococcal species, Haemophilus species, Salmonella species, Streptococcal species, Legionella species and Mycobacterium species. Protozoan infections encompassed by the invention include, but are not restricted to, those caused by Plasmodium species, Leishmania species, Trypanosoma species, Toxoplasma species, Entamoeba species and Giardia species. Helminth infections may include, but are not limited to, infections caused by Schistosoma species. Fungal infections contemplated by the present invention include, but are not limited to, infections caused by Histoplasma species and Candida species.

Suitable autoimmune disorders include, but are not limited to, autoimmune rheumatologic disorders (such as, for example, rheumatoid arthritis, Sjogren's syndrome, scleroderma, lupus such as systemic lupus erythematosus (SLE) and lupus nephritis, polymyositis-dermatomyositis, cryoglobulinemia, anti-phospholipid antibody syndrome and psoriatic arthritis), autoimmune gastrointestinal and liver disorders (such as, for example, inflammatory bowel diseases e.g., ulcerative colitis and Crohn's disease, autoimmune gastritis and pernicious anemia, autoimmune hepatitis, primary biliary cirrhosis, primary sclerosing cholangitis and celiac disease), vasculitis (such as, for example, anti-neutrophil cytoplasmic antibody (ANCA)-negative vasculitis and ANCA-associated vasculitis, including Churg-Strauss vasculitis, Wegener's granulomatosis and microscopic polyangiitis), autoimmune neurological disorders (such as, for example, multiple sclerosis, opsoclonus myoclonus syndrome, myasthenia gravis, neuromyelitis optica, Parkinson's disease, Alzheimer's disease and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, and autoimmune endocrine disorders (such as, for example, diabetic-related autoimmune diseases such as type I diabetes mellitis, Addison's disease and autoimmune thyroid disease (e.g. Graves' disease and thyroiditis)).

Suitable respiratory disorders include, but are not limited to, chronic obstructive pulmonary disease (COPD) or asthma especially allergic asthma.

In some embodiments, the methods further comprise detecting overexpression of a PD-1, PD-L1 and/or PD-L2 gene in a tumor sample obtained from the subject, wherein the tumor sample comprises the cancer stem cell tumor cells and optionally the non-cancer stem cell tumor cells, prior to administering the proteinaceous molecule of the invention to the subject.

In particular embodiments, any one of the methods described above involve the administration of one or more further active agents as described in Section 4 supra, such as an additional cancer therapy and/or an anti-infective agent, especially an additional cancer therapy.

The proteinaceous molecules of the invention, particularly a proteinaceous molecule corresponding to residues 255 to 271 of PD-L1, especially a proteinaceous molecule of Formula 1, any one of SEQ ID NO: 1-21, especially any one of SEQ ID NO: 1-18 or variant proteinaceous molecule as described herein, are useful for inhibiting or reducing the acetylation of a polypeptide. In some embodiments, the acetylation is catalyzed by an acetyltransferase; especially a histone acetyltransferase. In some embodiments, the histone acetyltransferase is GCNS, Hat1, ATF-2, Tip60, MOZ, MORF, HBO1, p300, CBP, SRC-1, ACTR, TIF-2, SRC-3, TAF1, TFIIIC and/or CLOCK; especially p300.

Thus, in a further aspect of the invention, there is provided a method of inhibiting the catalytic activity of an acetyltransferase in a subject, comprising administering a proteinaceous molecule comprising, consisting or consisting essentially of an amino acid sequence corresponding to an acetylation site, embodiments of which are described herein. The invention also extends to a use of a proteinaceous molecule described herein for inhibiting the catalytic activity of an acetyltransferase in a subject, and a proteinaceous molecule described herein for use in inhibiting the catalytic activity of an acetyltransferase in a subject. In preferred embodiments, the acetyltransferase is a histone acetyltransferase, embodiments of which are described above.

The present invention also contemplates a method of producing a proteinaceous molecule that inhibits or reduces nuclear localization of a nuclear localizable polypeptide wherein acetylation of an acetylation site of the nuclear localizable polypeptide increases its nuclear localization in a cell, the method comprising:
a) contacting a cell with a proteinaceous molecule comprising, consisting or consisting essentially of an amino acid sequence corresponding to an acetylation site; and
b) detecting a reduction in or inhibition of the nuclear localization of the nuclear localizable polypeptide in the cell relative to a normal or reference level of nuclear localization in the absence of the proteinaceous molecule.

In some embodiments, the proteinaceous molecule is a fragment of a nuclear localizable polypeptide. In particular embodiments, the proteinaceous molecule comprises, consists or consists essentially of 50, 45, 40, 35, 30, 25, 20, 19, 18 or 17 (and each integer therebetween) amino acid residues or less.

In some embodiments, the amino acid sequence corresponding to an acetylation site is an amino acid sequence corresponding to residues 255 to 271 of PD-L1. In particular embodiments, the proteinaceous molecule is distinguished from PD-L1 by the addition, deletion and/or substitution of at least one (e.g. 1, 2, 3, 4, 5, 6, 7, 8, 9, etc.) amino acid in residues 255 to 271 of PD-L1. In some embodiments, the proteinaceous molecule is distinguished from PD-L1 by the addition, deletion and/or substitution of 1 to 8, 1 to 7, 1 to 6, 1 to 5, 1 to 4, 1 to 3, or 1 to 2 amino acids in residues 255 to 271 of PD-L1.

In another aspect, the present invention provides a method of producing a proteinaceous molecule that inhibits or reduces nuclear localization of a nuclear localizable polypeptide wherein acetylation of an acetylation site of the nuclear localizable polypeptide increases its nuclear localization in a cell, the method comprising:
a) contacting a cell with a proteinaceous molecule comprising, consisting or consisting essentially of an amino acid sequence corresponding to residues 255 to 271 of PD-L1; and
b) detecting a reduction in or inhibition of the nuclear localization of the nuclear localizable polypeptide in the cell relative to a normal or reference level of nuclear localization in the absence of the proteinaceous molecule.

In some embodiments, the proteinaceous molecule is distinguished from PD-L1 by the addition, deletion and/or substitution of at least one (e.g. 1, 2, 3, 4, 5, 6, 7, 8, 9, etc.) amino acid in residues 255 to 271 of PD-L1. In some embodiments, the proteinaceous molecule is distinguished from PD-L1 by the addition, deletion and/or substitution of 1 to 8, 1 to 7, 1 to 6, 1 to 5, 1 to 4, 1 to 3, or 1 to 2 amino acids in residues 255 to 271 of PD-L1.

A reduction in or inhibition of the nuclear localization of the nuclear localizable polypeptide may be determined using standard techniques in the art, non-limiting examples of which include immunofluorescence, immunohistochemistry staining, chromatin immunoprecipitation (ChIP), ChIP-seq, chromatin accessibility assays such as DNase-seq, FAIRE-seq and ATAC-seq assays, such as that described in Satelli, et al. (2016) Sci Rep, 6:28910; Bajetto, et al. (2000) Brain Research Protocols, 5(3): 273-281; and Sung, et al. (2014) BMC Cancer, 14:951, the entire contents of which are incorporated by reference.

In yet another aspect, there is provided a method of producing a proteinaceous molecule that inhibits or reduces at least one of formation, proliferation, viability or EMT of a cancer stem cell, the method comprising:
a) contacting a cancer stem cell with a proteinaceous molecule comprising, consisting or consisting essentially of an amino acid sequence corresponding to residues 255 to 271 of PD-L1; and
b) detecting a reduction in or inhibition of the formation, proliferation or EMT of the cancer stem cell relative to a normal or reference level of formation, proliferation, viability or EMT of the cell in the absence of the proteinaceous molecule.

In some embodiments, the proteinaceous molecule is distinguished from PD-L1 by the addition, deletion and/or substitution of at least one (e.g. 1, 2, 3, 4, 5, 6, 7, 8, 9, etc) amino acid in residues 255 to 271 of PD-L1. In some embodiments, the proteinaceous molecule is distinguished from PD-L1 by the addition, deletion and/or substitution of 1 to 8, 1 to 7, 1 to 6, 1 to 5, 1 to 4, 1 to 3, or 1 to 2 amino acids in residues 255 to 271 of PD-L1.

The amino acid sequence of the proteinaceous molecule may correspond to a natural, designed or synthetic acetylation site. In some embodiments, the acetylation site is a site of a nuclear localizable polypeptide, such as, but not limited to, PD-1, PD-L1 or PD-L2. Suitable acetylation sites are as previously described herein. In other embodiments, the amino acid sequence corresponding to an acetylation site is other than an amino acid sequence of a nuclear localizable polypeptide e.g. PD-1, PD-L1 and/or PD-L2.

A proteinaceous molecule with an amino acid sequence corresponding to a designed acetylation site may be identified using medicinal chemistry techniques standard in the art.

An acetylation site of a polypeptide may be identified using computational methods such as that described in Hake and Janzen (2013) Protein Acetylation: Methods and Protocols, Methods in Molecular Biology, vol. 981; Li, et al. (2014) Sci Rep, 4:5765; Hou, et al. (2014) PLoS One, 9(2):e89575; and Wuyun, et al. (2016) PLoS One, 11(5):e0155370 (the entire contents of which are incorporated by reference); or can be determined experimentally using, for example, mutagenesis of predicted residues in combination with detection of levels of acetylation using, for example, an antibody directed to an acetylated amino acid residue such as an acetylated lysine. The involvement of surrounding and/or proximal residues may be determined using medicinal chemistry techniques standard in the art.

A skilled person would be well aware of suitable assays used to evaluate the nuclear localization of a polypeptide, such as PD-1, PD-L1 and/or PD-L2, and to identify proteinaceous molecules that inhibit or reduce the nuclear localization of a polypeptide, such as PD-1, PD-L1 and/or PD-L2. Screening for active agents according to the invention can be achieved by any suitable method. For example, the method may include contacting a cell expressing a polynucleotide corresponding to a gene that encodes the polypeptide of interest, such as PD-1, PD-L1 and/or PD-L2, with an agent suspected of having the inhibitory activity and screening for the inhibition or reduction of the level of the polypeptide of interest in the nucleus of the cell. Alternatively, the inhibition of the functional activity of the polypeptide of interest or the lowering of the level of a transcript encoded by the polynucleotide, or the inhibition of the activity or expression of a downstream cellular target of the polypeptide or of the transcript, may be screened wherein the activity is related to nuclear localization of the polypeptide of interest. Detecting such inhibition may be achieved utilizing techniques including, but not limited to, ELISA, immunofluorescence, Western blots, immunoprecipitation, immunostaining, slot or dot blot assays, scintillation proximity assays, fluorescent immunoassays using antigen-binding molecule conjugates or antigen conjugates of fluorescent substances such as fluorescein or rhodamine, RIA, Ouchterlony double diffusion analysis, immunoassays employing an avidin-biotin or a streptavidin-biotin detection system, nucleic acid detection assays including reverse transcriptase polymerase chain reaction (RT-PCR), cell proliferation assays such as a WST-1 proliferation assay and immunoblot analysis of cells treated with PD-L1 Half-Way ChIP. The acetylation of a polypeptide may be determined using an antibody directed to the acetylated polypeptide, such as an antibody directed to an acetylated lysine residue.

It will be understood that a polynucleotide from which a polypeptide of interest, such as PD-1, PD-L1 and/or PD-L2, is regulated or expressed may be naturally occurring in the cell which is the subject of testing or it may have been introduced into the host cell for the purposes of testing.

The inhibition of the catalytic activity of an acetylase may be determined using techniques standard in the art. For example, the inhibition of an acetyltransferase may be assessed using a fluorescence assay such as the acetyltransferase activity assay kit from Abcam (Catalogue number ab204536), the p300 fluorogenic assay kit from BPS Bioscience (Catalogue number 50092), or the p300 inhibitor screening assay kit (fluorometric) from Abcam (Catalogue number ab196996); a colorimetric assay such as the histone acetyltransferase activity assay kit from Abcam (Catalogue number ab65352); or a chemiluminescence assay such as the p300 chemiluminescent assay kit from BPS Bioscience (Catalogue number 50077).

These methods provide a mechanism for performing high throughput screening of putative modulatory agents such as proteinaceous agents comprising synthetic, combinatorial, chemical and natural libraries.

Active molecules may be further tested in the animal models to identify those molecules having the most potent in vivo effects. These molecules may serve as lead molecules for the further development of pharmaceuticals by, for example, subjecting the compounds to sequential modifications, molecular modeling and other routine procedures employed in rational drug design.

In order that the invention may be readily understood and put into practical effect, particular preferred embodiments will now be described by way of the following non-limiting examples.

### EXAMPLES

### Example 1—Localization of PD-L1 in Metastasis Initiating Cells (MICs) from Breast Cancer and Melanoma Patients

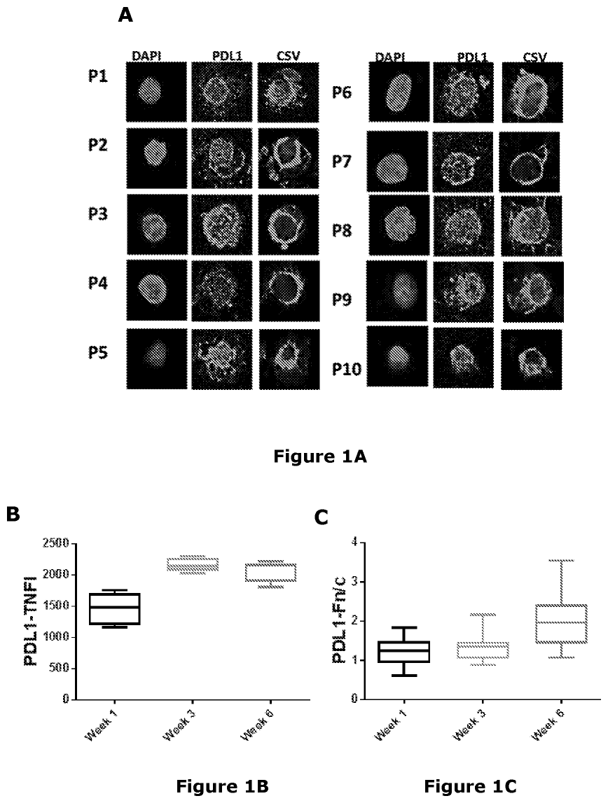

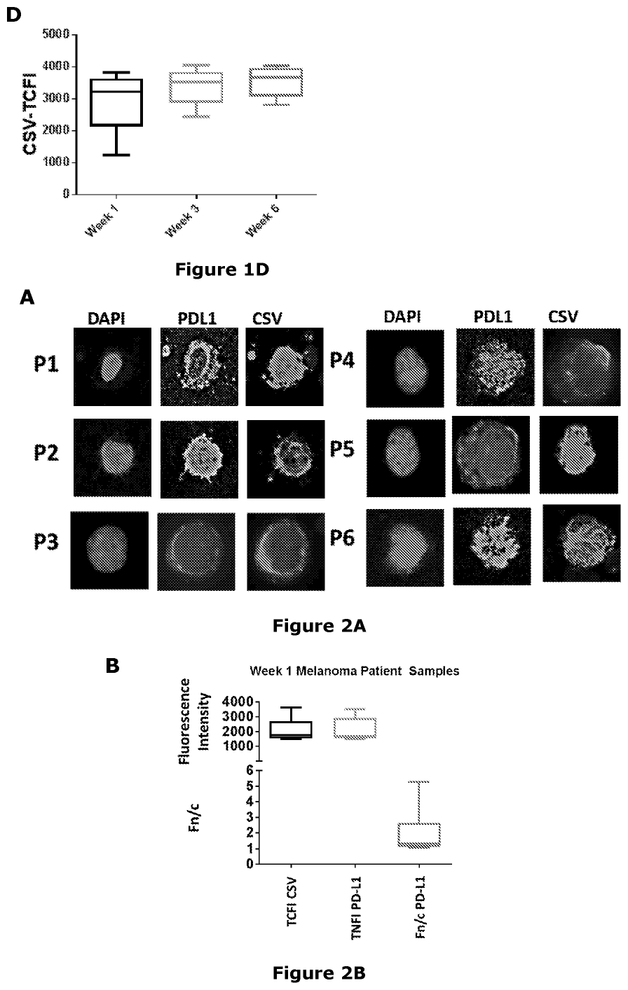

Confocal laser scanning microscopy was performed on MICs isolated from liquid biopsies from metastatic breast cancer and melanoma patients. PD-L1 showed significant nuclear localization in both breast cancer (FIGS. 1A to 1D) and melanoma (FIGS. 2A and 2B) cells as indicated by a strong TNFI and a Fn/c score of greater than one.

### Example 2—Localization of PD-L1 in Breast Cancer Cells

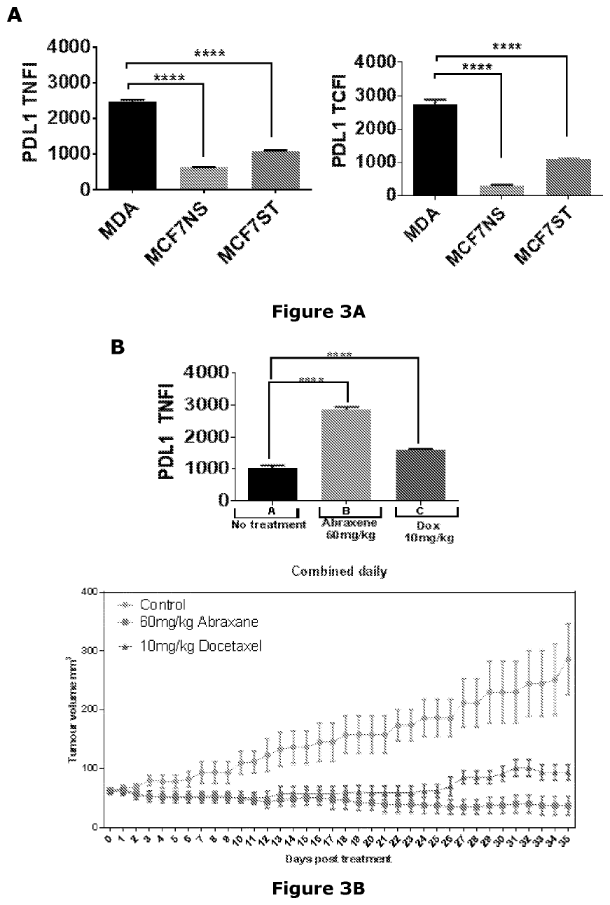

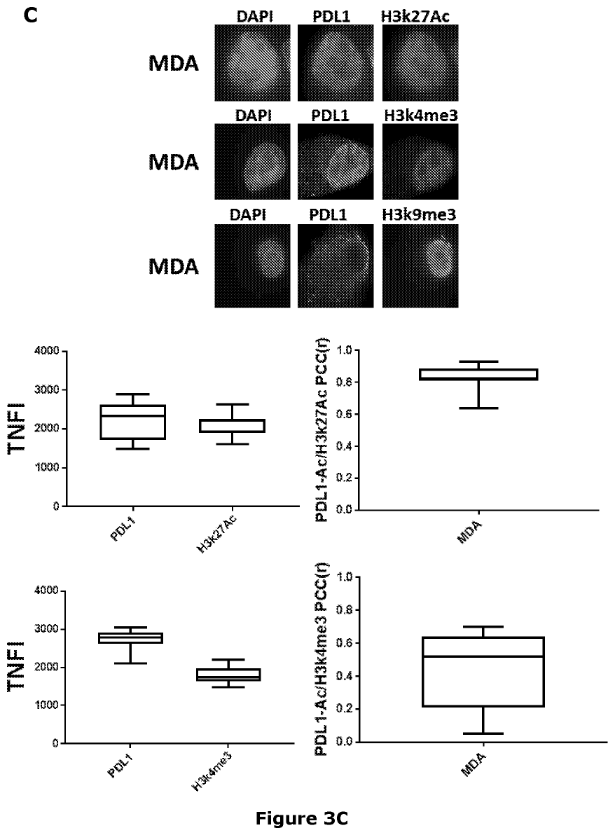

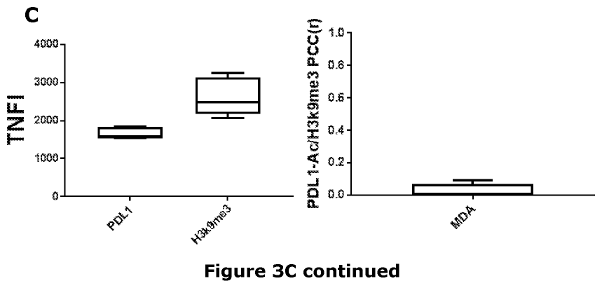

Confocal laser scanning microscopy was performed on MDA-MB-231 (MDA) cells and epithelial (MCF7 NS) or mesenchymal (MCF7 ST) MCF7 cells to examine the localization of PD-L1. PD-L1 was detectable in MDA-MB-231 and epithelial and mesenchymal MCF7 cells, with high nuclear localization in mesenchymal MCF7 cells and MDA-MB-231 cells in particular (FIG. 3A).

The expression of PD-L1 in mouse MDA-MB-231 xenografts treated for 35 days with abraxane (60 mg/kg) or docetaxel (10 mg/kg) was investigated using confocal laser scanning microscopy. Surviving, resistant MDA-MB-231 xenograft cells treated with abraxane or docetaxel expressed higher levels of PD-L1 in the nucleus compared with untreated cells (FIG. 3B).

The localization of PD-L1 and the key histone markers, acetylated H3K27 (H3K27ac), trimethylated H3K4 (H3K4me3), and trimethylated H3K9 (H3K9me3), was investigated in MDA-MB-231 cells using confocal laser scanning microscopy. PD-L1 colocalized with the active markers H3K27ac and H3K4me3, but not the repressive marker H3K9me3 in the nucleus of the cells (FIG. 3C).

### Example 3—Effect of K263Q Mutation on Localization of PD-L1 and Expression of Tumor Cell Markers

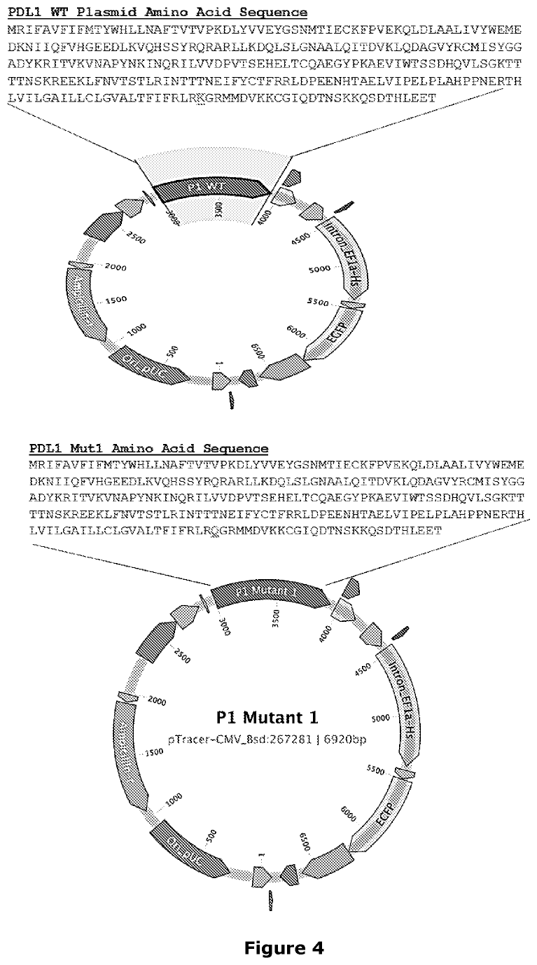

Residues 255-271 of PD-L1 were identified as a methylation and acetylation site, with lysine 263 being the methylated/acetylated residue, using high stringency methylation prediction software described in Wen, et al. (2016) Bioinformatics, 32(20): 3107-3115 and acetylation prediction software described in Li, et al. (2014) Sci Rep, 4:5765. To determine the role of acetylation of this site on localization of PD-L1 and expression of tumor cell markers, MCF7 cells were transfected with a plasmid containing the wild-type PD-L1 sequence and a plasmid containing a PD-L1 [K263Q] mutant sequence (Mut1) (FIG. 4). Lysine 263 was replaced with glutamine to prevent acetylation at this position.

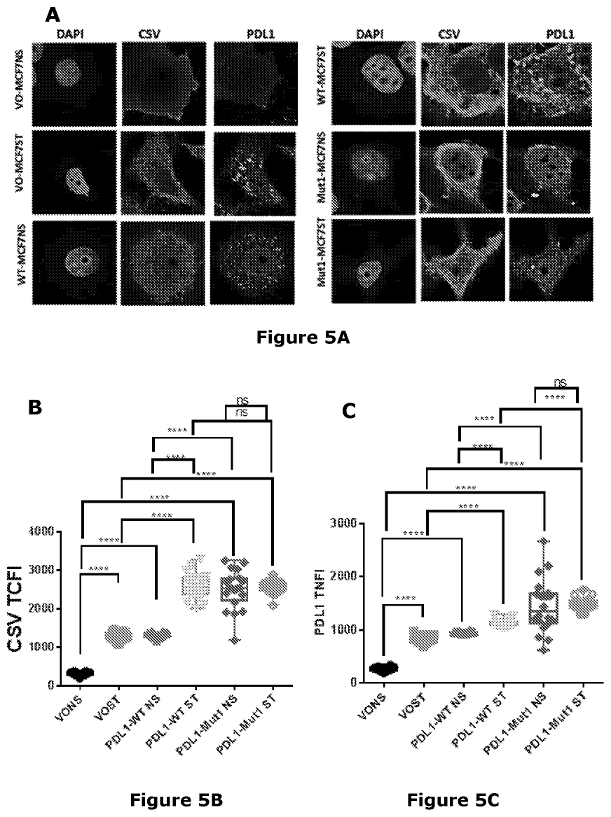

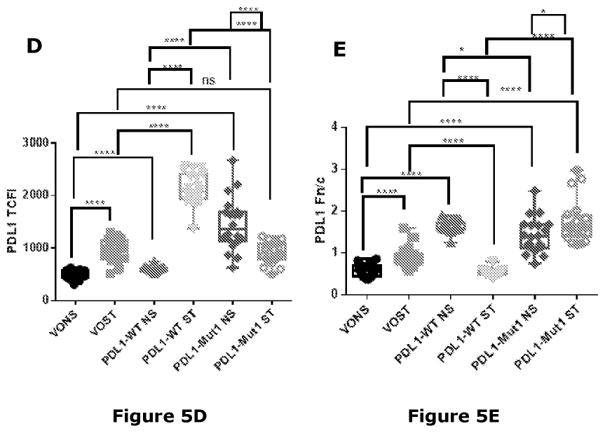

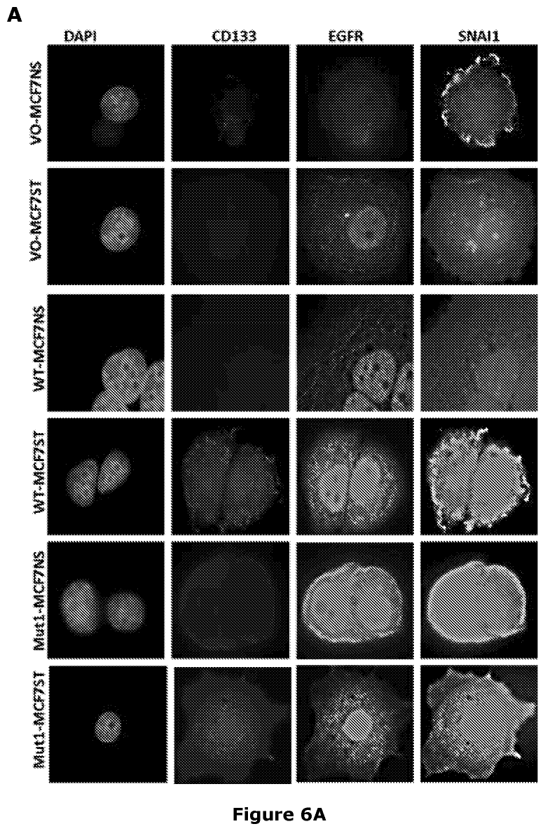

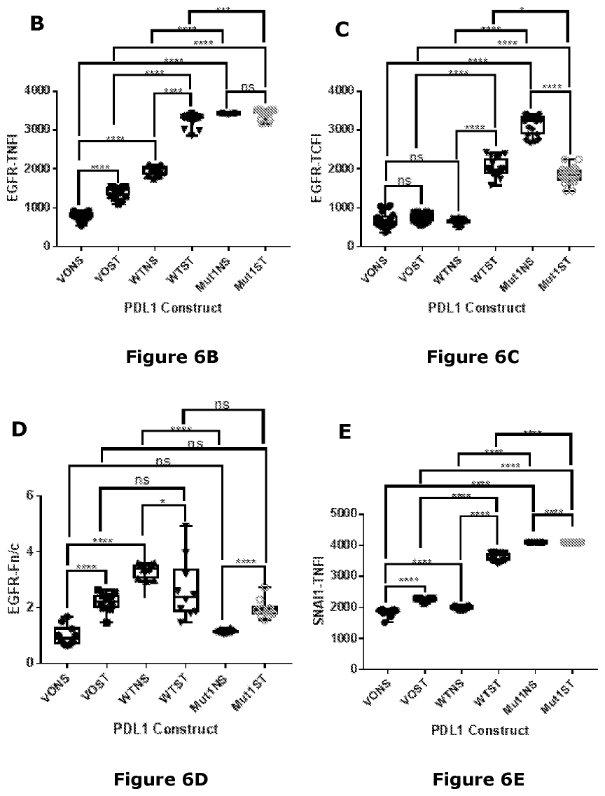

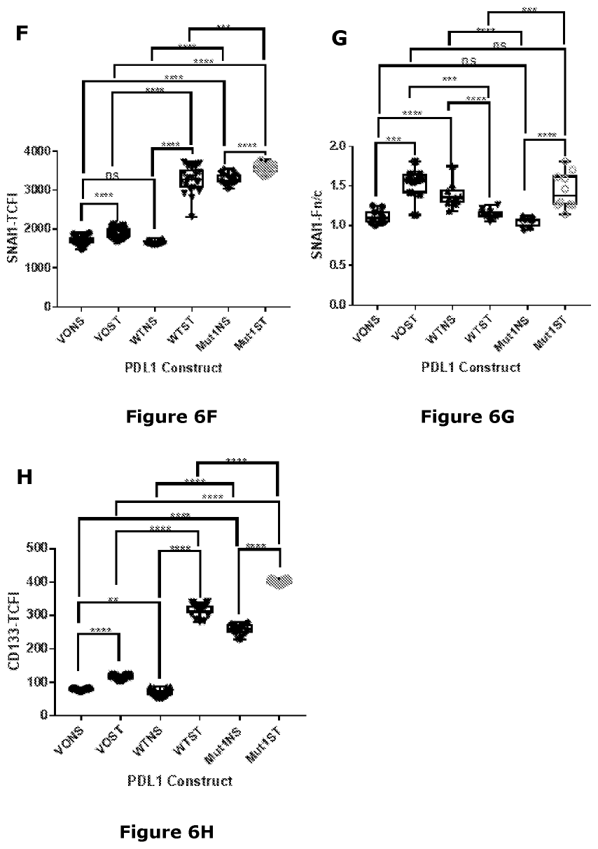

Overexpression of PD-L1 in the cells transfected with the plasmid containing the wild-type PD-L1 sequence resulted in increased expression of cell surface vimentin (CSV) which is a marker for aggressive tumor cells (FIGS. 5A and 5B), CD133 which is a marker for chemo-persistent cancer stem cells (FIGS. 6A and 6H), and the mesenchymal markers, EGFR (FIGS. 6A to 6D) and SNAI1 (FIGS. 6A and 6E to 6G) when compared with cells transfected with the empty vector. PD-L1 nuclear localization was also increased in cells transfected with the plasmid containing the wild-type PD-L1 sequence (FIGS. 5A and 5C to 5E). Cells transfected with the Mut1 plasmid displayed significantly increased expression of CSV when compared with cells transfected with the empty vector, and significantly increased expression of CD133, EGFR and SNAI1 when compared with cells transfected with the empty vector and the plasmid containing the wild-type PD-L1 sequence (FIGS. 5A, 5B and 6A to 6H). Strikingly, the cells transfected with the Mut1 plasmid displayed significantly increased nuclear localization of PD-L1 when compared to the cells transfected with the empty vector or the plasmid containing the wild-type PD-L1 sequence (FIGS. 5A and 5C to 5E).

In addition to significantly increasing expression of all mesenchymal markers investigated, the cells transfected with the plasmid containing the wild-type PD-L1 sequence and the Mut1 plasmid also suggested the acquisition of a motile phenotype, indicating a strong metastatic potential. This was particularly evident for cells transfected with the Mut1 plasmid.

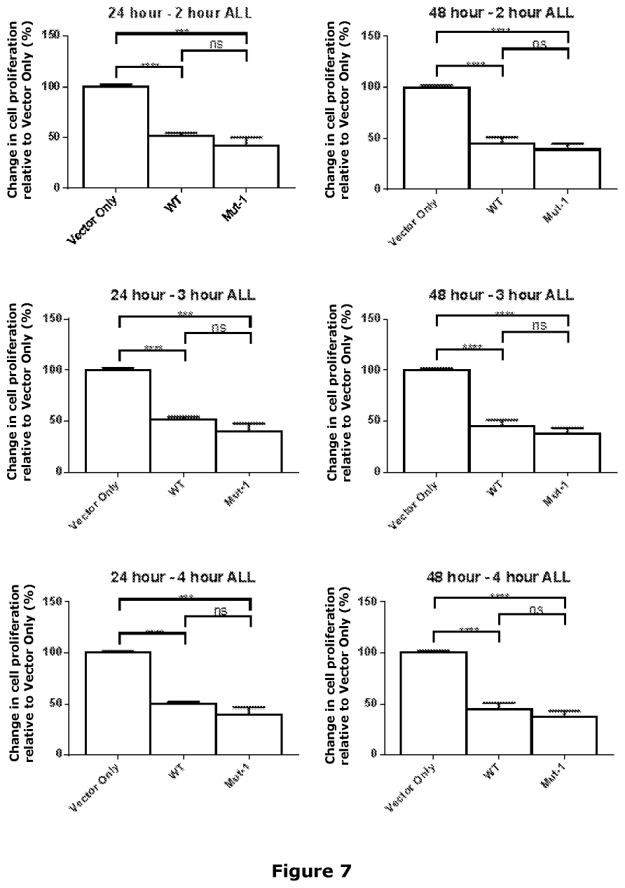

The effect of the PD-L1 [K263Q] mutation on cell proliferation was investigated using a WST-1 proliferation assay. Transfection of MCF7 cells with the plasmid containing the wild-type PD-L1 sequence caused a significant inhibition of cell proliferation (FIG. 7). This inhibition was increased when cells were transfected with the Mut1 plasmid (FIG. 7). In combination with the microscopy data, this suggests that the transfected cells are acquiring a metastatic, mesenchymal, non-proliferative status.

Thus, it is evident that lysine 263 plays a critical role in the nuclear localization of PD-L1 and nuclear localization of PD-L1 is important for regulating tumor markers of aggressiveness and a mesenchymal, cancer stem cell-like, chemo-persistent, non-proliferative state.

### Example 4—Localization of Trimethylated and Acetylated PD-L1 in MDA-MB-231 Cells and in Circulating Tumor Cells from Breast Cancer and Melanoma Patients

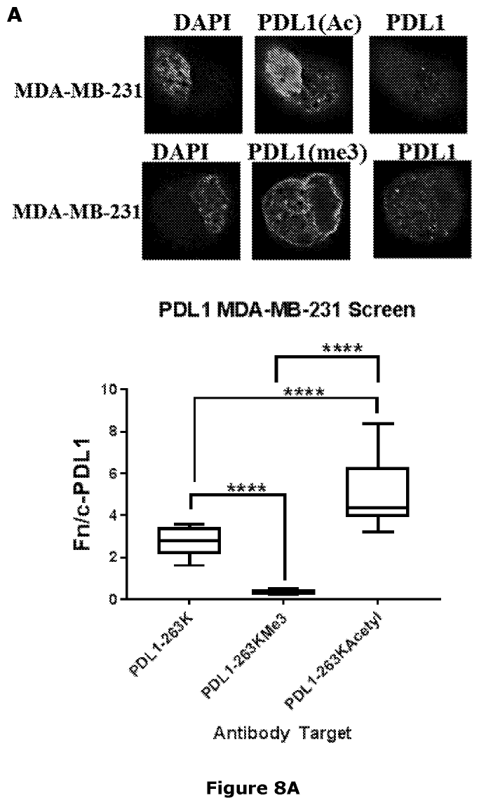

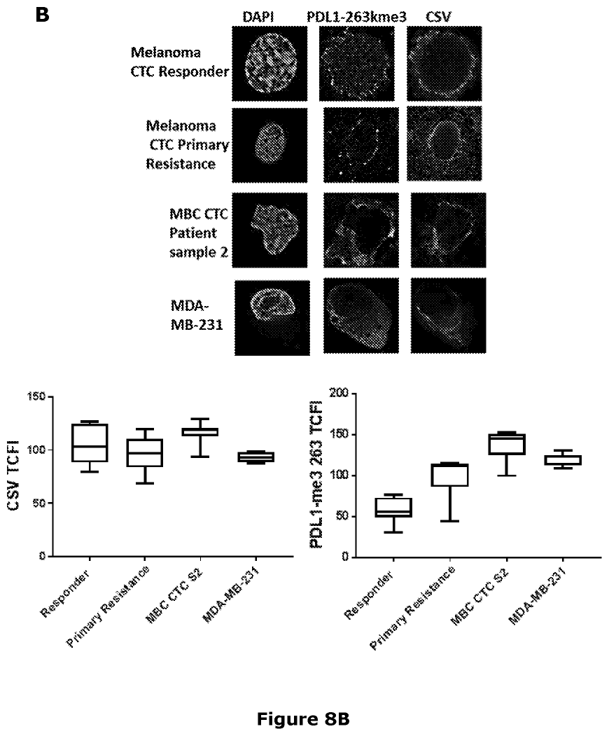

Confocal laser scanning microscopy was used to examine the localization of PD-L1 trimethylated at lysine 263 (‘trimethylated PD-L1’) and PD-L1 acetylated at lysine 263 (‘acetylated PD-L1’) in MDA-MB-231 cells using antibodies specific for PD-L1 (obtained from Santa-Cruz), PD-L1 acetylated at lysine 263 and PD-L1 trimethylated at lysine 263. Acetylated PD-L1 (PDL1-263KAcetyl) demonstrated a clear nuclear presence evidenced by a high Fn/c (ratio of nuclear to cytoplasmic fluorescence) whereas trimethylated PD-L1 (PDL1-263KMe3) was predominantly located in the cytoplasm of MDA-MB-231 cells, indicated by a low Fn/c (FIG. 8A).

The localization of trimethylated and acetylated PD-L1 was then examined in non-permeabilized MDA-MB-231 cells, circulating tumor cells (CTCs) isolated from metastatic breast cancer patients (MBC CTC S1 or S2), CTCs isolated from melanoma patients which responded to treatment with chemotherapeutics (responder), and CTCs isolated from melanoma patients with primary (primary resistance) or secondary resistance (2^{nd} resistance) to treatment with chemotherapeutics. Only trimethylated PD-L1 labelled clearly in these cells (FIG. 8B), whereas acetylated PD-L1 had almost no binding (FIG. 8C), indicating that acetylated PD-L1 is predominantly located in the nucleus, whereas trimethylated PD-L1 is predominantly located in the cytoplasm or at the cell surface.

### Example 5—Synthesis of P1, P2 And P3

P1, P2 and P3 (refer to Table 4) were designed based on the methylation site of PD-L1. P1, P2 and P3 were synthesized using automated modern solid phase peptide synthesis and purification technology using the mild Fmoc chemistry method, for example, as described in as described in Ensenat-Waser, et al. (2002) IUBMB Life, 54:33-36 and WO 2002/010193. Couplings were performed using standard N,N′-diisopropylcarbodiimide (DIC)/hydroxybenzotriazole (HOBt) coupling. Following deprotection, peptides were purified using automated preparative reversed phase-high performance liquid chromatography (RP-HPLC). Fractions were analyzed using analytical RP-HPLC and mass spectrometry. Fractions of 98% purity or higher were combined to give the final product.

All peptides tested were myristoylated through the N-terminal amino group of the N-terminal amino acid. Myristoylation was carried out by covalently coupling myristic acid to the N-terminal residue using standard DIC/HOBt coupling as described above, prior to deprotection and purification of the peptides.

TABLE 4
P1, 2 and 3 sequences

| Peptide | Sequence |
|---|---|
| P1 | Myristoyl-LTFIFRLRKGRMMDVKK-NH₂ |
| P2 | Myristoyl-LTFIFRLRQGRMMDVKK-NH₂ |
| P3 | Myristoyl-LTFIFRLRK(Ac)GRMMDVKK-NH₂ |

### Example 6—Effect of P1, P2 and P3 on Nuclear Expression of PD-L1 and Acetylated PD-L1 in MDA-MB-231 Cells

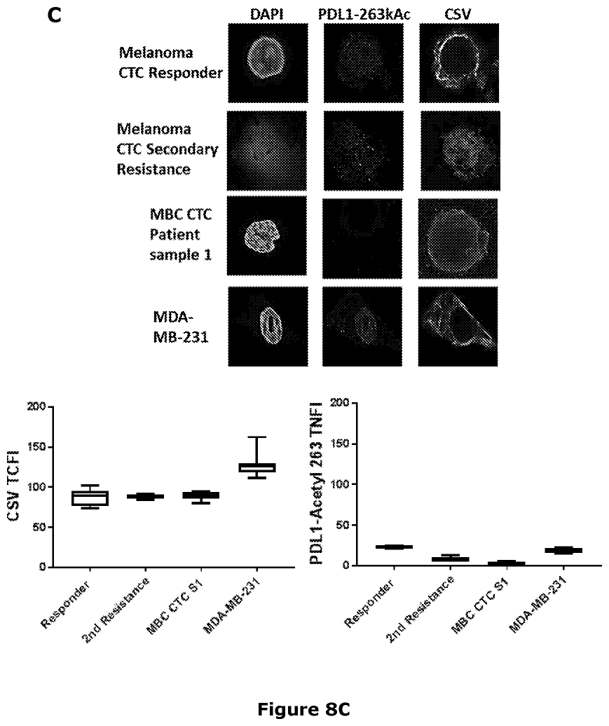

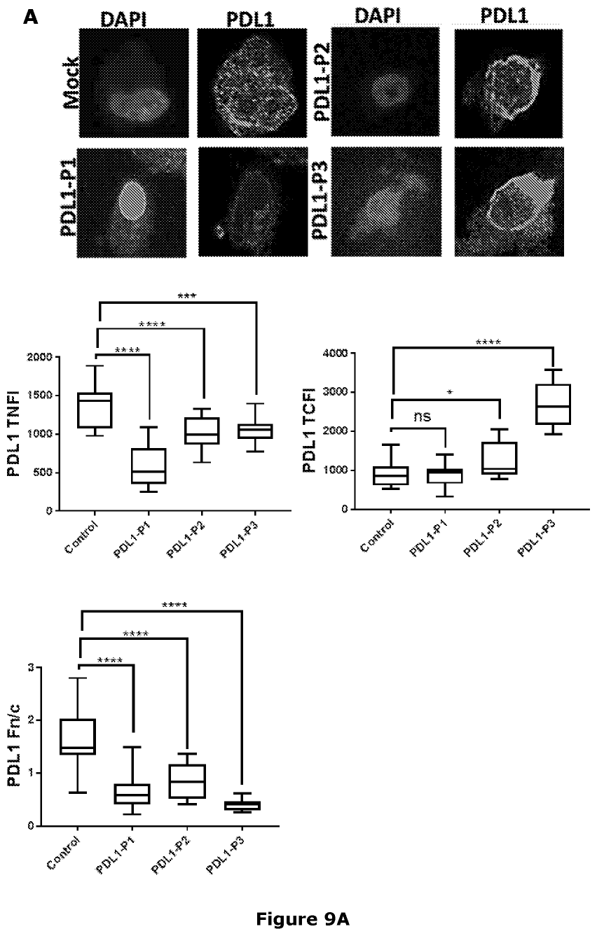

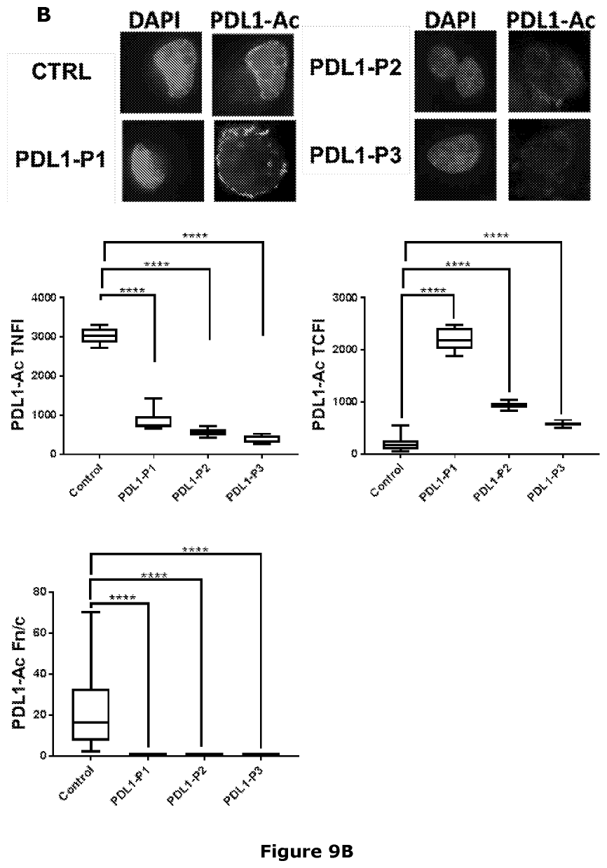

Confocal laser scanning microscopy was employed to investigate the effect of P1, P2 and P3 (synthesized in accordance with Example 5) on the nuclear expression of PD-L1 and acetylated PD-L1 (PDL1-Ac) (FIG. 9). The localization of PD-L1 was significantly biased towards the cytoplasm, indicated by a low Fn/c (FIG. 9A). P1, P2 and P3 inhibited the nuclear expression of PD-L1. The localization of acetylated PD-L1 was predominantly restricted to the nucleus (FIG. 9B). Strikingly, the total nuclear fluorescence intensity (TNFI) was significantly reduced in cells treated with P1, P2 and P3, with these cells displaying cytoplasmic expression of acetylated PD-L1. The Fn/c was significantly reduced for cells treated with P1, P2 and P3, indicating that acetylated PD-L1 was biased towards the cytoplasm as a result of treatment with P1, P2 and P3.

In order to investigate whether an N-terminally truncated peptide corresponding to a possible nuclear localization signal inhibits the nuclear localization of PD-L1, P4 was designed and synthesized in accordance with the procedure of Example 5.

TABLE 5
P4 peptide sequence

| Peptide | Sequence |
|---|---|
| P4 | Myristoyl-FRLRKGRMMDVKK-NH₂ |

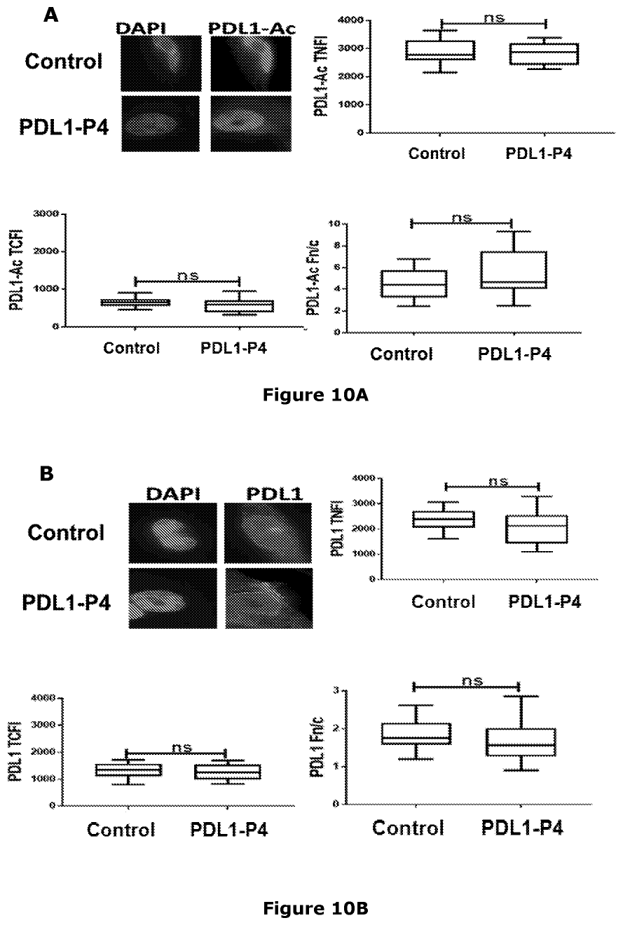

P4 had no effect on the localization of PD-L1 or acetylated PD-L1 (FIG. 10A and 10B).

### Example 7—Effect of P1, P2 and P3 on the Cancer Stem Cell Phenotype (CD44^{high}/CD24^{low}) in MDA-MB-231 Cells

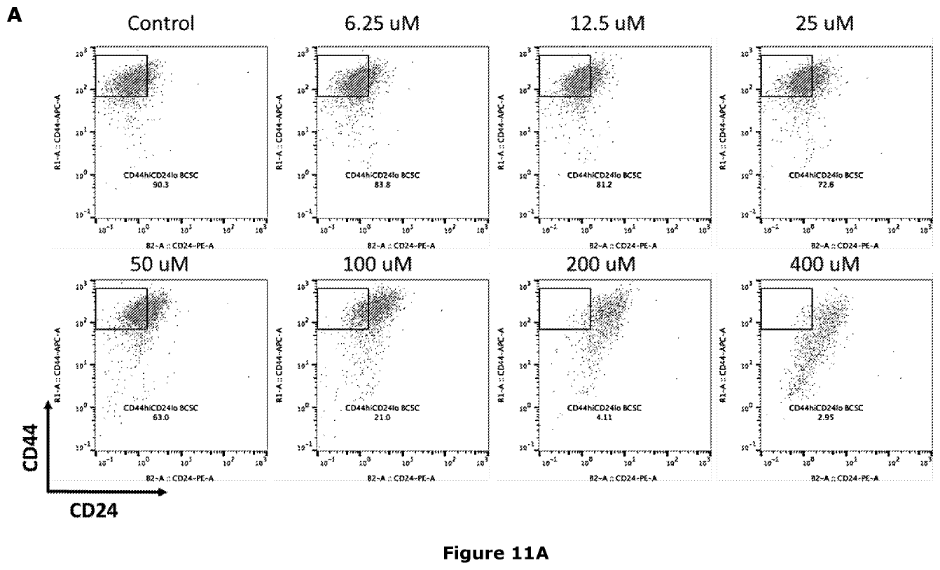

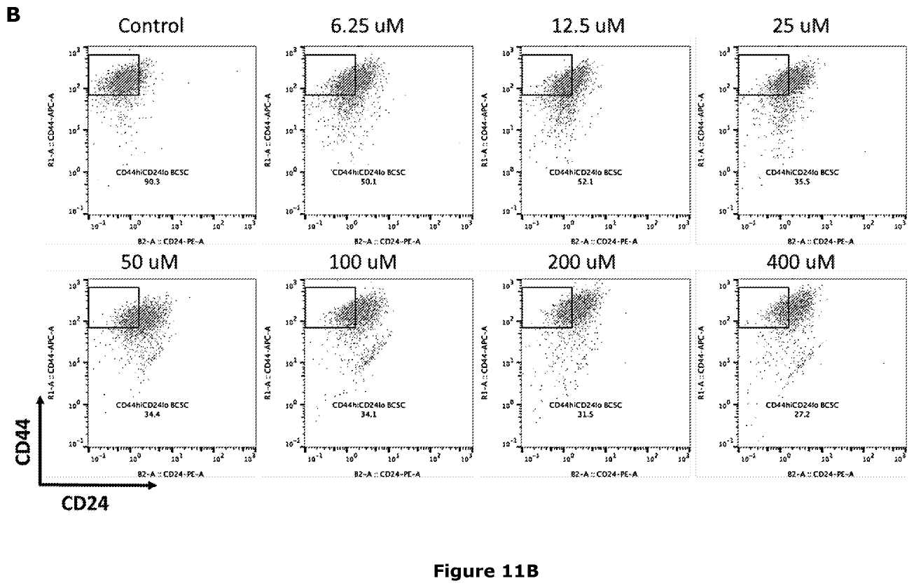

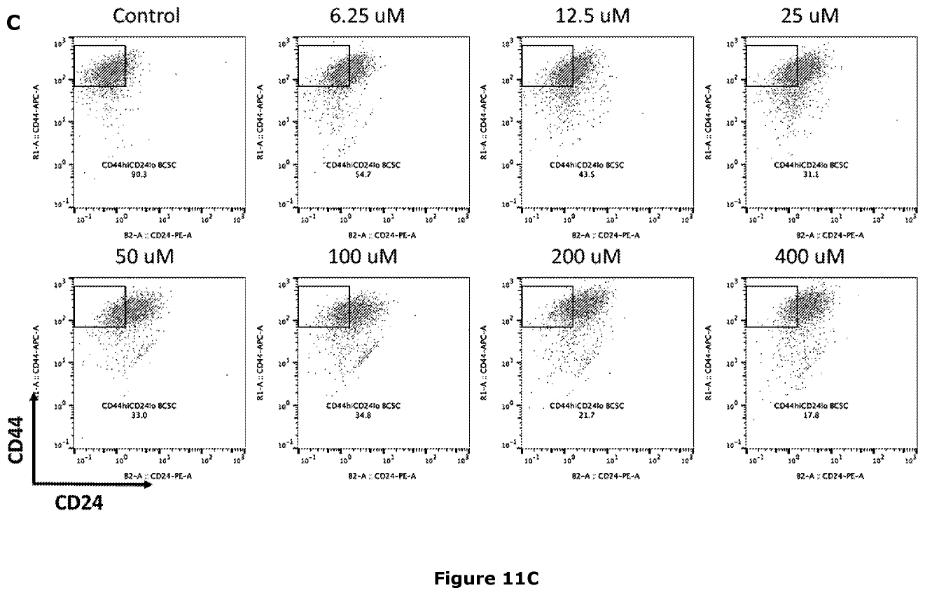

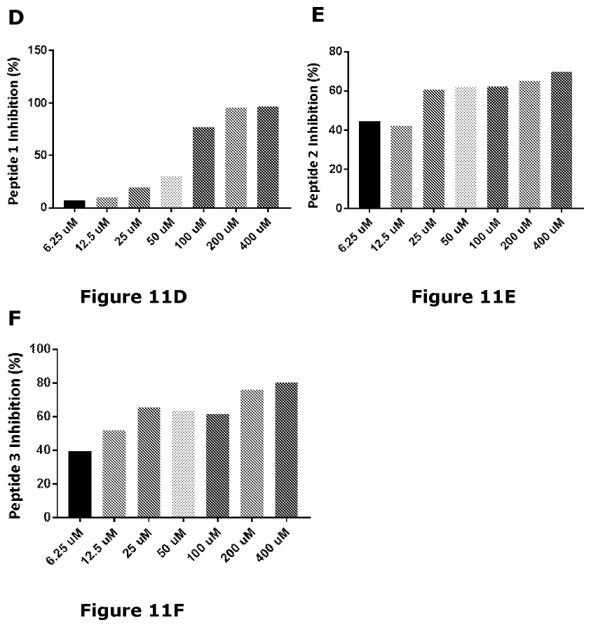

FACS analysis was employed to determine the effect of treatment with P1, P2 and P3 (synthesized in accordance with Example 5) on the cancer stem cell phenotype (CD44^{high}/CD24^{low}) in MDA-MB-231 cells (which have a high cancer stem cell phenotype). Treatment with P1, P2 and P3 significantly inhibited the cancer stem cell phenotype, with P3 causing the greatest inhibition of the cancer stem cell phenotype (FIGS. 11A to 11F).

### Example 8—Effect of P1, P2 and P3 on MDA-MB-231 Cell Proliferation

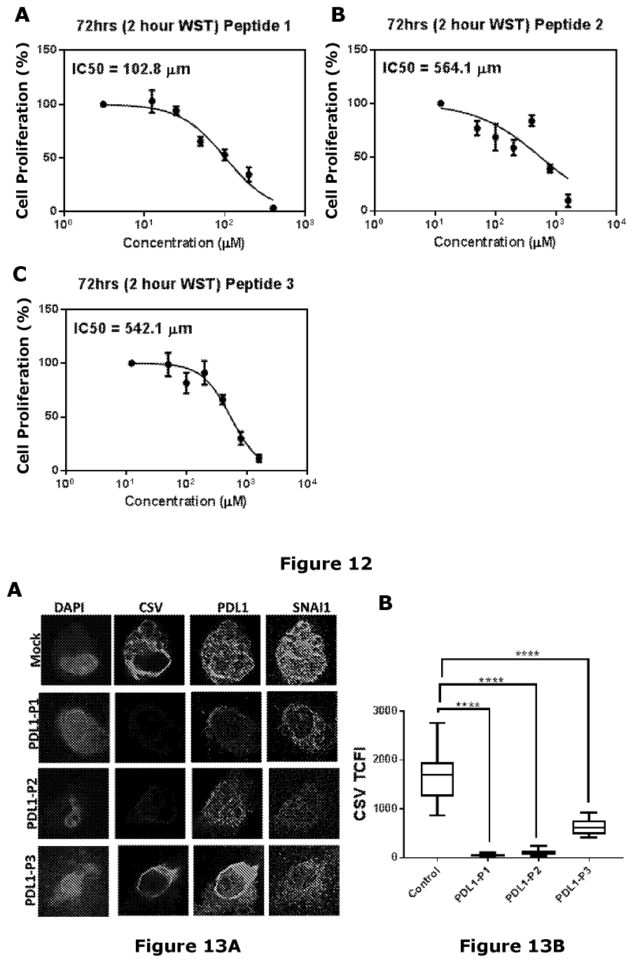

The effect of P1, P2 and P3 (synthesized in accordance with Example 5) on MDA-MB-231 cell proliferation was assessed using a WST-1 proliferation assay. Despite overexpression of PD-L1 which promotes a chemo-persistent, non-proliferative phenotype, P1, P2 and P3 all inhibited proliferation of MDA-MB-231 cells, with IC₅₀ values of 102.8 μM, 564.1 μM and 542.1 μM, respectively (FIGS. 12A to 12C).

### Example 9—Effect of P1, P2 and P3 on Expression of Tumor Cell Markers and Epigenetic Enzymes

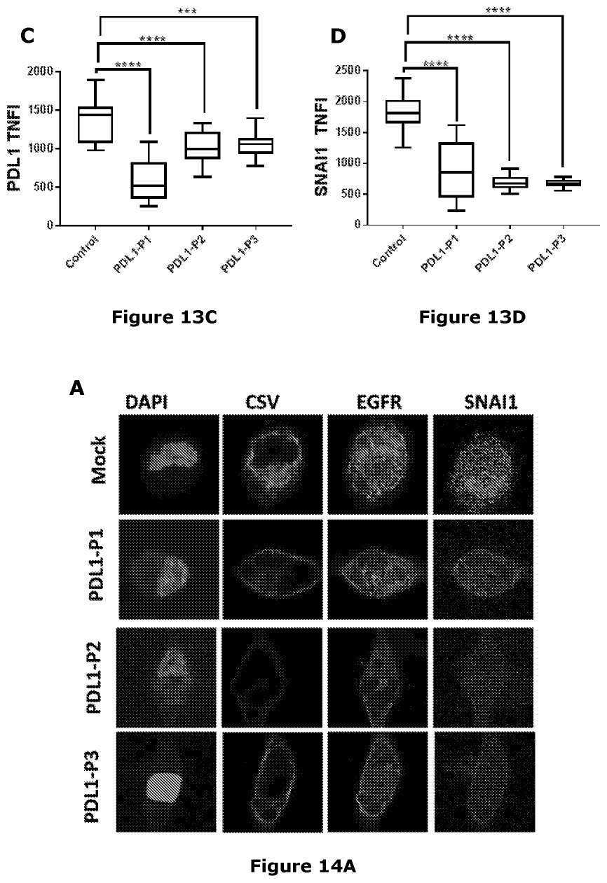

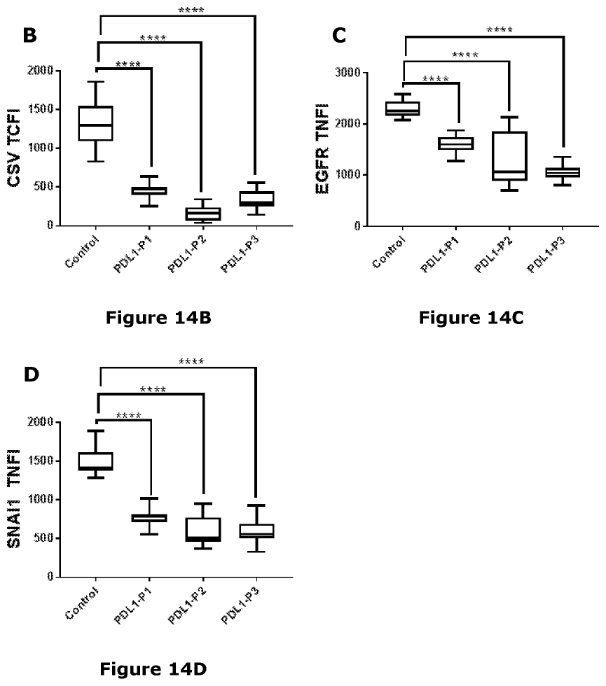

The effect of P1, P2 and P3 (synthesized in accordance with Example 5) on the expression of PD-L1, CSV, SNAI1 and EGFR in MDA-MB-231 cells was assessed using confocal laser scanning microscopy. PD-L1 nuclear localization was significantly inhibited for P1, P2 and P3 (FIGS. 13A and 13C). In addition, P1, P2 and P3 uniformly inhibited the expression of CSV (FIGS. 13A, 13B, 14A and 14B) which is a marker for aggressive tumor cells, and mesenchymal markers EGFR (FIGS. 14A and 14C) and SNAI1 (FIGS. 13A, 13D, 14A and 14D). These results confirm that P1, P2 and P3 significantly inhibit nuclear localization of PD-L1 and demonstrates the importance of nuclear PD-L1 in mesenchymal metastasis initiating cancer cells.

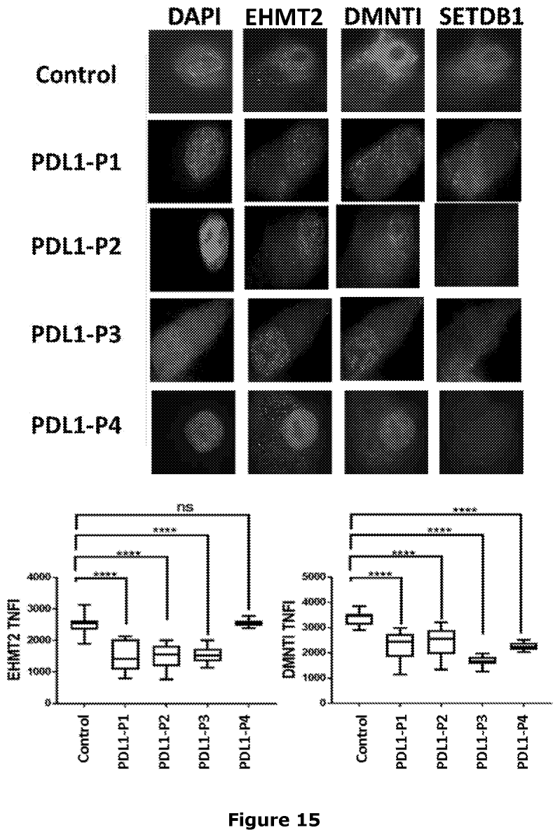

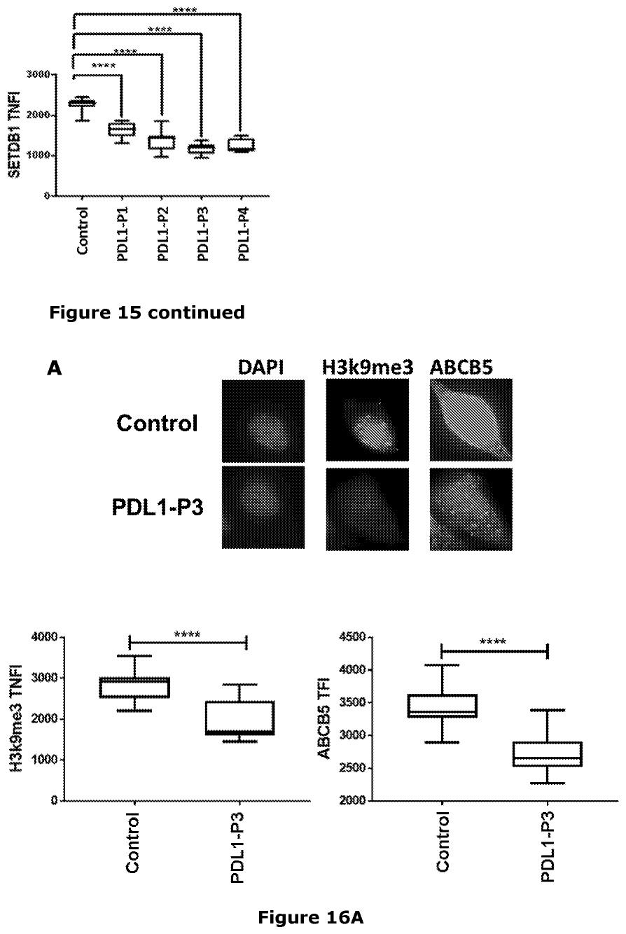

The effect of P1, P2, P3 and P4 (synthesized in accordance with Example 5) on the expression of EHTM2, DMNTI and SETDB1 in MDA-MB-231 cells was determined using confocal laser scanning microscopy. P1, P2 and P3 significantly abrogated expression of EHTM2, DMNTI and SETDB1, which are epigenetic enzymes implicated in cancer progression (FIG. 15). P4 also inhibited nuclear localization of DMNTI and SETDB1 but did not have a significant effect on EHTM2 localization.

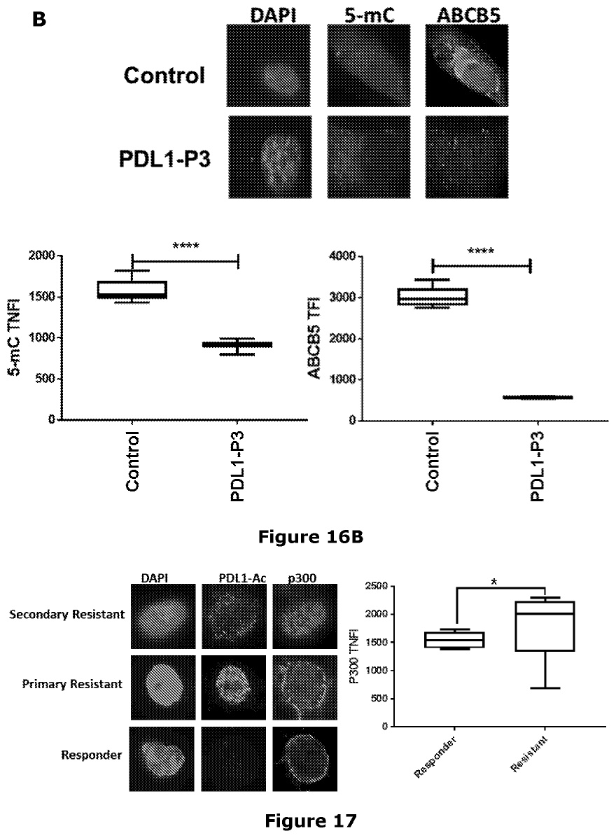

Following on from these results, the effect of P3 on the expression of H3K9me3 (the target of SETDB1), 5-methylcytosine (as an indicator of DNA methylation) and ABCBS (a marker for resistance) in MDA-MB-231 cells was investigated using confocal laser scanning microscopy. P3 strongly inhibited expression of H3K9me3, 5-methylcytosine and ABCBS (FIGS. 16A and 16B).

### Example 10—Coexpression of Acetylated PD-L1 and P300 in Melanoma and Breast Cancer

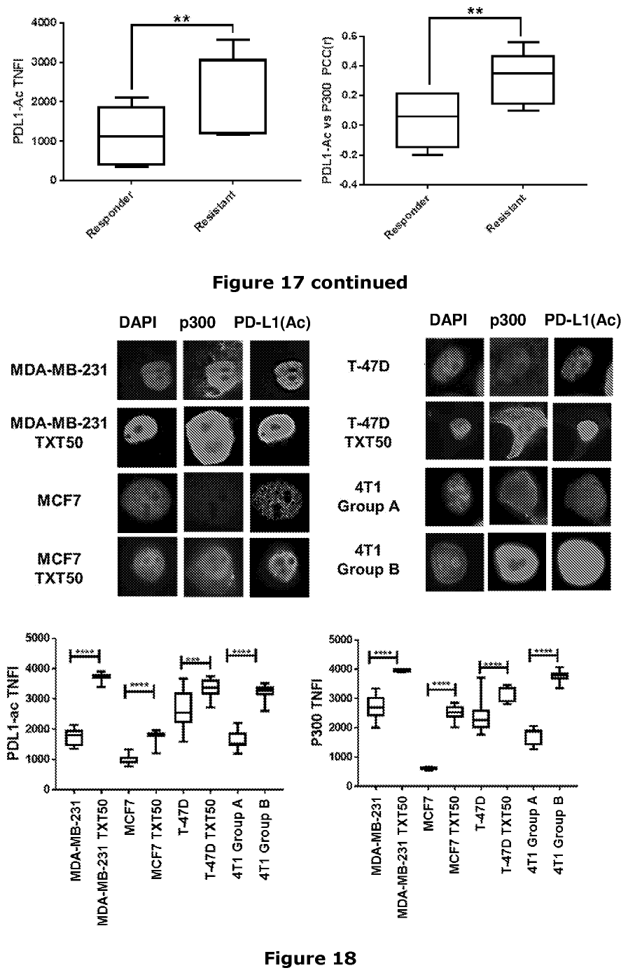

The expression of acetylated PD-L1 and the acetyltransferase, p300, was investigated in CTCs from metastatic melanoma patients using confocal laser scanning microscopy. Acetylated PD-L1 was enriched in the nucleus of metastatic melanoma CTCs displaying primary and secondary resistance to immunotherapy (FIG. 17). Expression of p300 was slightly increased in metastatic melanoma CTCs displaying resistance to immunotherapy. Interestingly, the Pearson correlation coefficient (PCC(r)) of p300 and acetylated PD-L1 is significantly increased in the resistant CTCs, with little or no PCC(r) in the CTCs which respond to immunotherapy, indicating that acetylated PD-L1 and p300 colocalize in resistant CTCs.

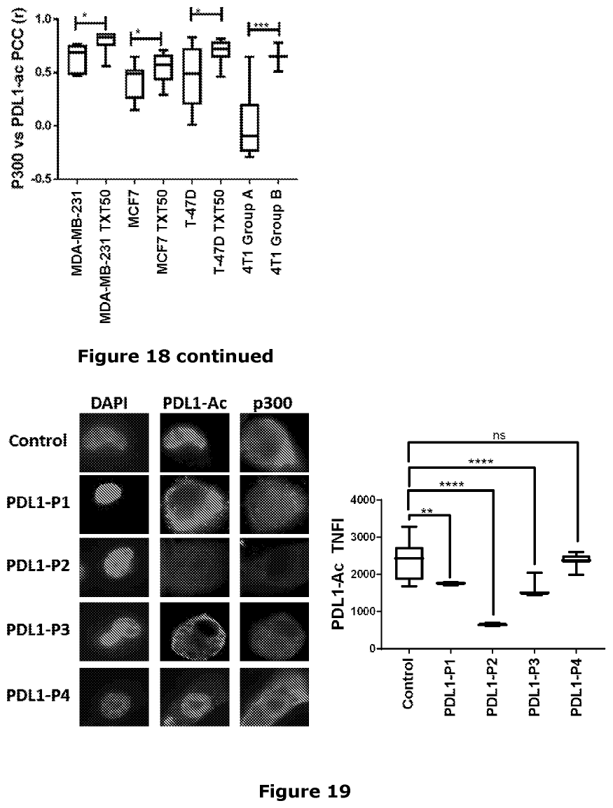

The expression of p300 and acetylated PD-L1 was further investigated in docetaxel resistant metastatic breast cancer cell lines (MDA-MB-231, MCF7 and T-47D) and abraxane resistant metastatic breast cancer cell lines(4T1 cells) using confocal laser scanning microscopy. Nuclear expression of acetylated PD-L1 was significantly increased in resistant cells (MDA-MB-231 TXT50, MCF7 TXT50, T-47D TXT50 and 4T1 Group B) compared with non-resistant cells (MDA-MB-231, MCF7, T-47D and 4T1 Group A) (FIG. 18). Similarly to acetylated PD-L1, nuclear expression of p300 was significantly increased in resistant cells compared with non-resistant cells (FIG. 18). Acetylated PD-L1 and p300 strongly co-localized within the nucleus of the cells, with this significantly increasing in the resistant cells (FIG. 18).

### Example 11—Effect of P1, P2, P3 and P4 on P300 Localization

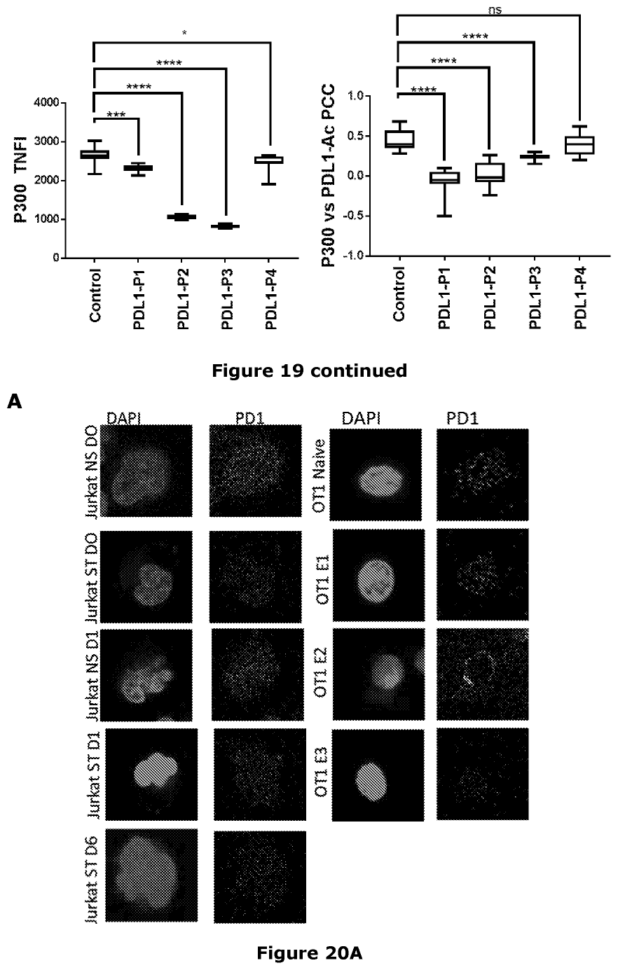

The effect of P1, P2, P3 and P4 (synthesized in accordance with Example 5) on p300 localization was examined using confocal laser scanning microscopy. Similarly to the effect on acetylated PD-L1, P1, P2 and P3 significantly decreased p300 nuclear expression, with P3 having the greatest effect (FIG. 19). These peptides also significantly decreased the colocalization of acetylated PD-L1 and p300. P4 slightly decreased nuclear expression of p300 but had no effect on the colocalization of acetylated PD-L1 and p300.

### Example 12—Localization of Pd-1 in Jurkat T-Cells or OT1 Derived T-Cells

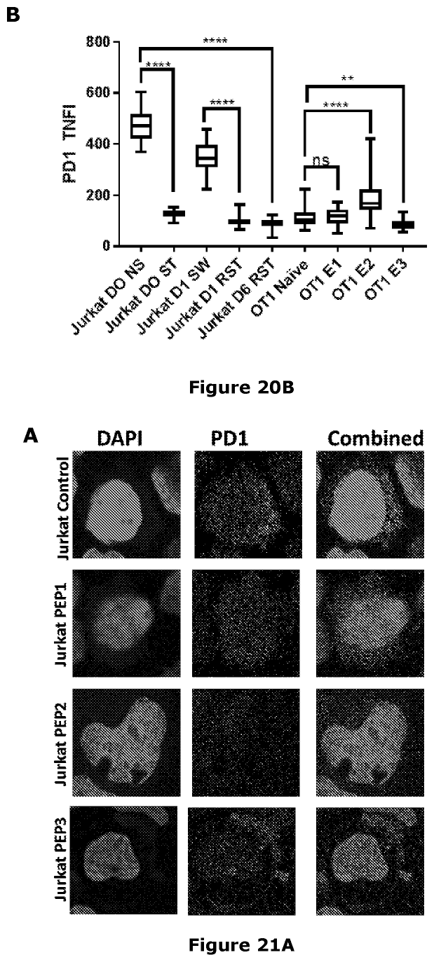

The localization of PD-1 in Jurkat T-cells or OT1 derived T-cells was determined using confocal laser scanning microscopy. PD-1 nuclear localization was evident in Jurkat T-cells (a leukemic cell line) (FIGS. 20A and 20B). However, expression was reduced upon activation of the inflammatory pathway. Nuclear PD-1 localization was also evident in OT1 derived naïve and effector T-cells, but at a lower intensity than in Jurkat T-cells.

### Example 13—Effect of P1, P2, and P3 on PD-1 Localization in Jurkat T-Cells

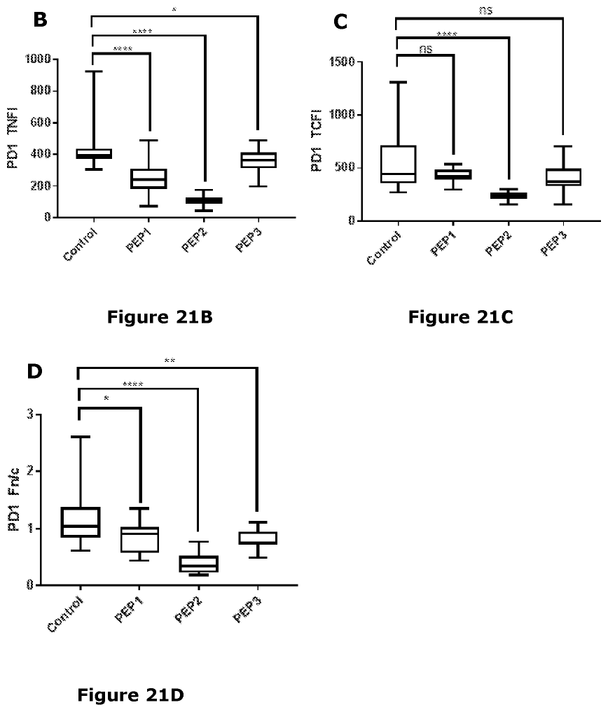

The effect of P1, P2 and P3 (synthesized in accordance with Example 5) on the localization of PD-1 in Jurkat T-cells was assessed using confocal laser scanning microscopy. PD-1 nuclear localization in Jurkat T-cells was significantly inhibited by P1, P2 and P3 relative to the control (FIGS. 21A to 21D).

### Example 14—Effect of P1, P2 and P3 on PD-L2 Localization in MDA-MB-231 Cells

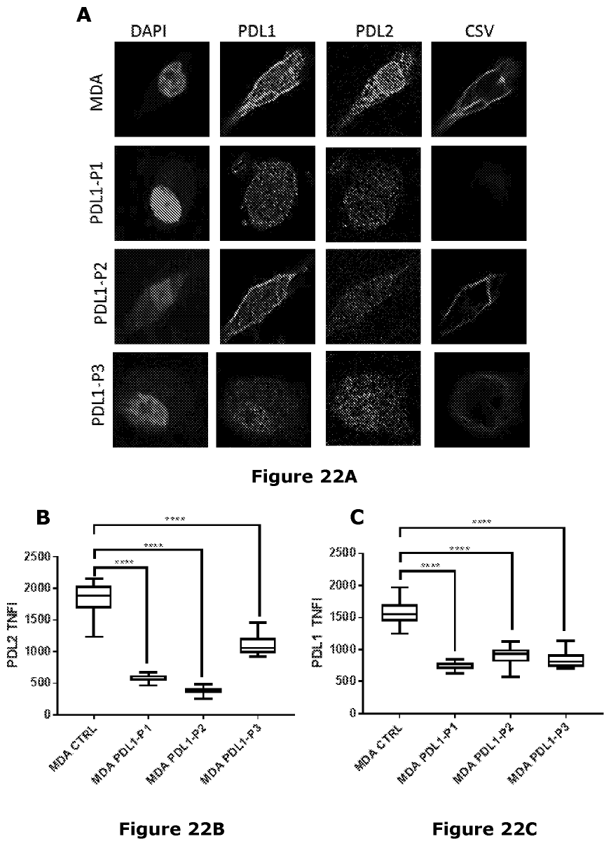

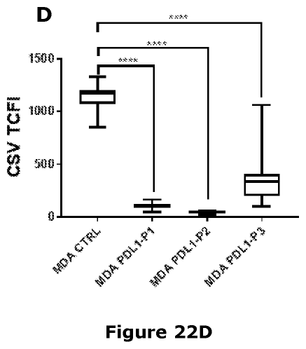

The effect of P1, P2 and P3 (synthesized in accordance with Example 5) on the localization of PD-L2 in MDA-MB-231 cells was determined using confocal laser scanning microscopy. PD-L2 nuclear localization was significantly inhibited by P1, P2 and P3 relative to the control (FIGS. 22A and 22B). Again, P1, P2 and P3 significantly inhibited nuclear localization of PD-L1 (FIGS. 22A and 22C) and CSV expression which is a marker for aggressive tumor cells (FIGS. 22A and 22D), relative to the control.

### Example 15—Effect of P1 Analogues on Cancer Stem Cells in MDA-MB-231 Cells

Analogues of P1 (Table 6) were designed and synthesized in accordance with the procedure of Example 5. The ability of these analogues to inhibit cancer stem cells was determined using FACS analysis on MDA-MB-231 cells, which constitutively contain approximately 90% CD44^{hi}CD24^{lo} cancer stem cells, treated with the peptides.

TABLE 6
Peptide analogues

| Peptide | Mutation | Sequence |
|---|---|---|
| P1 | N/A | Myristoyl-LTFIFRLRKGRMMDVKK-NH₂ |
| P2 | N/A | Myristoyl-LTFIFRLRQGRMMDVKK-NH₂ |
| P3 | N/A | Myristoyl-LTFIFRLRK(Ac)GRMMDVKK-NH₂ |
| P4 | N/A | Myristoyl-FRLRKGRMMDVKK-NH₂ |
| 2815302 | P1₂₋₁₇[T2A] | Myristoyl-AFIFRLRKGRMMDVKK-NH₂ |
| 2815303 | P1₂₋₁₇[F3A] | Myristoyl-TAIFRLRKGRMMDVKK-NH₂ |
| 2815304 | P1₂₋₁₇[I4A] | Myristoyl-TFAFRLRKGRMMDVKK-NH₂ |
| 2815305 | P1₂₋₁₇[F5A] | Myristoyl-TFIARLRKGRMMDVKK-NH₂ |
| 2815306 | P1₂₋₁₇[R6A] | Myristoyl-TFIFALRKGRMMDVKK-NH₂ |
| 2815307 | P1₂₋₁₇[L7A] | Myristoyl-TFIFRARKGRMMDVKK-NH₂ |
| 2815308 | P1₂₋₁₇[R8A] | Myristoyl-TFIFRLAKGRMMDVKK-NH₂ |
| 2815309 | P1₂₋₁₇[K9A] | Myristoyl-TFIFRLRAGRMMDVKK-NH₂ |
| 2815310 | P1₂₋₁₇[G10A] | Myristoyl-TFIFRLRKARMMDVKK-NH₂ |
| 2815318 | P1₂₋₁₇[R11A] | Myristoyl-TFIFRLRKGAMMDVKK-NH₂ |
| 2815312 | P1₂₋₁₇[M12A] | Myristoyl-TFIFRLRKGRAMDVKK-NH₂ |
| 2815313 | P1₂₋₁₇[M13A] | Myristoyl-TFIFRLRKGRMADVKK-NH₂ |
| 2815314 | P1₂₋₁₇[D14A] | Myristoyl-TFIFRLRKGRMMAVKK-NH₂ |
| 2815315 | P1₂₋₁₇[V15A] | Myristoyl-TFIFRLRKGRMMDAKK-NH₂ |
| 2815316 | P1₂₋₁₇[K16A] | Myristoyl-TFIFRLRKGRMMDVAK-NH₂ |
| 2815317 | P1₂₋₁₇[K17A] | Myristoyl-TFIFRLRKGRMMDVKA-NH₂ |
| 2853801 | P1[L1A] | Myristoyl-ATFIFRLRKGRMMDVKK-NH₂ |
| 2853802 | P1[L1E] | Myristoyl-ETFIFRLRKGRMMDVKK-NH₂ |
| 2853803 | P1[L1K] | Myristoyl-KTFIFRLRKGRMMDVKK-NH₂ |
| 2853804 | P1[T2E] | Myristoyl-LEFIFRLRKGRMMDVKK-NH₂ |
| 2853805 | P1[T2K] | Myristoyl-LKFIFRLRKGRMMDVKK-NH₂ |
| 2853806 | P1[T2L] | Myristoyl-LLFIFRLRKGRMMDVKK-NH₂ |
| 2853807 | P1[F3E] | Myristoyl-LTEIFRLRKGRMMDVKK-NH₂ |
| 2853808 | P1[F3K] | Myristoyl-LTKIFRLRKGRMMDVKK-NH₂ |
| 2853809 | P1[F3V] | Myristoyl-LTVIFRLRKGRMMDVKK-NH₂ |
| 2853810 | P1[I4E] | Myristoyl-LTFEFRLRKGRMMDVKK-NH₂ |
| 2853811 | P1[I4K] | Myristoyl-LTFKFRLRKGRMMDVKK-NH₂ |
| 2853812 | P1[I4L] | Myristoyl-LTFLFRLRKGRMMDVKK-NH₂ |
| 2853813 | P1[F5E] | Myristoyl-LTFIERLRKGRMMDVKK-NH₂ |
| 2853814 | P1[F5Y] | Myristoyl-LTFIYRLRKGRMMDVKK-NH₂ |
| 2853815 | P1[F5H] | Myristoyl-LTFIHRLRKGRMMDVKK-NH₂ |
| 2853816 | P1[F5L] | Myristoyl-LTFILRLRKGRMMDVKK-NH₂ |
| 2853817 | P1[R6E] | Myristoyl-LTFIFELRKGRMMDVKK-NH₂ |
| 2853818 | P1[R6V] | Myristoyl-LTFIFVLRKGRMMDVKK-NH₂ |
| 2853819 | P1[L7F] | Myristoyl-LTFIFRFRKGRMMDVKK-NH₂ |
| 2853820 | P1[L7V] | Myristoyl-LTFIFRVRKGRMMDVKK-NH₂ |
| 2853821 | P1[L7E] | Myristoyl-LTFIFRERKGRMMDVKK-NH₂ |
| 2853822 | P1[L7K] | Myristoyl-LTFIFRKRKGRMMDVKK-NH₂ |
| 2853823 | P1[R8E] | Myristoyl-LTFIFRLEKGRMMDVKK-NH₂ |
| 2853824 | P1[R8K] | Myristoyl-LTFIFRLKKGRMMDVKK-NH₂ |
| 2853825 | P1[R8L] | Myristoyl-LTFIFRLLKGRMMDVKK-NH₂ |
| 2853826 | P1[K9Orn(Ac)] | Myristoyl-LTFIFRLROrn(Ac)GRMMDVKK-NH₂ |
| 2853827 | P1[G10D] | Myristoyl-LTFIFRLRKDRMMDVKK-NH₂ |
| 2853828 | P1[G10K] | Myristoyl-LTFIFRLRKKRMMDVKK-NH₂ |
| 2853829 | P1[G10V] | Myristoyl-LTFIFRLRKVRMMDVKK-NH₂ |
| 2853830 | P1[R11E] | Myristoyl-LTFIFRLRKGEMMDVKK-NH₂ |
| 2853831 | P1[R11V] | Myristoyl-LTFIFRLRKGVMMDVKK-NH₂ |
| 2853832 | P1[M12E] | Myristoyl-LTFIFRLRKGREMDVKK-NH₂ |
| 2853833 | P1[M12K] | Myristoyl-LTFIFRLRKGRKMDVKK-NH₂ |
| 2853834 | P1[M13E] | Myristoyl-LTFIFRLRKGRMEDVKK-NH₂ |
| 2853835 | P1[M13K] | Myristoyl-LTFIFRLRKGRMKDVKK-NH₂ |
| 2853836 | P1[D14K] | Myristoyl-LTFIFRLRKGRMMKVKK-NH₂ |
| 2853837 | P1[D14V] | Myristoyl-LTFIFRLRKGRMMVVKK-NH₂ |
| 2853838 | P1[V15E] | Myristoyl-LTFIFRLRKGRMMDEKK-NH₂ |
| 2853839 | P1[V15K] | Myristoyl-LTFIFRLRKGRMMDKKK-NH₂ |
| 2853840 | P1[K16R] | Myristoyl-LTFIFRLRKGRMMDVRK-NH₂ |
| 2853841 | P1[K16E] | Myristoyl-LTFIFRLRKGRMMDVEK-NH₂ |
| 2853842 | P1[K16L] | Myristoyl-LTFIFRLRKGRMMDVLK-NH₂ |
| 2853843 | P1[K16Y] | Myristoyl-LTFIFRLRKGRMMDVYK-NH₂ |
| 2853844 | P1[K17R] | Myristoyl-LTFIFRLRKGRMMDVKR-NH₂ |
| 2853845 | P1[K17E] | Myristoyl-LTFIFRLRKGRMMDVKE-NH₂ |
| 2853846 | P1[K17L] | Myristoyl-LTFIFRLRKGRMMDVKL-NH₂ |
| 2853847 | P1[K17Y] | Myristoyl-LTFIFRLRKGRMMDVKY-NH₂ |

The FACS results are presented in Tables 7 and 8.

TABLE 7
Effect of peptide inhibitors on the number of CD44^{hi}CD24^{lo} CSCs in MDA-MB-231 cells (n = 3)

| | | | | | | | Average |
|---|---|---|---|---|---|---|---|
| | CD44^{hi}CD24^{lo} CSCs (%) | | | Inhibition of CSCs (%) | | | Inhibition of |
| Peptide | Repeat 1 | Repeat 2 | Repeat 3 | Repeat 1 | Repeat 2 | Repeat 3 | CSCs (%) |
| Ctrl | 89.62 | 89.72 | 89.74 | N/A | N/A | N/A | N/A |
| P1 | 5.98 | 9.00 | 8.72 | 93.33 | 89.97 | 90.28 | 91.19 |
| P2 | 18.73 | 15.56 | 12.62 | 79.10 | 82.65 | 85.94 | 82.56 |
| P3 | 6.48 | 7.99 | 10.90 | 92.77 | 91.10 | 87.85 | 90.57 |
| 2815302 | 9.24 | 7.36 | 4.73 | 89.69 | 91.79 | 94.73 | 92.07 |
| 2815303 | 21.23 | 16.92 | 17.09 | 76.31 | 81.14 | 80.95 | 79.47 |
| 2815304 | 50.53 | 46.84 | 43.79 | 43.62 | 47.79 | 51.20 | 47.54 |
| 2815305 | 83.18 | 80.00 | 87.10 | 7.19 | 10.83 | 2.94 | 6.99 |
| 2815306 | 29.28 | 27.66 | 26.21 | 67.33 | 69.17 | 70.79 | 69.09 |
| 2815307 | 67.40 | 66.80 | 56.59 | 24.79 | 25.54 | 36.94 | 29.09 |
| 2815308 | 43.29 | 49.27 | 44.44 | 51.70 | 45.08 | 50.47 | 49.08 |
| 2815309 | 0.00 | 0.00 | 0.00 | 100.00 | 100.00 | 100.00 | 100.00 |
| 2815310 | 54.84 | 54.37 | 52.63 | 38.81 | 39.40 | 41.35 | 39.85 |
| 2815318 | 40.51 | 40.61 | 39.86 | 54.80 | 54.73 | 55.59 | 55.04 |
| 2815312 | 0.00 | 0.00 | 0.00 | 100.00 | 100.00 | 100.00 | 100.00 |
| 2815313 | 69.60 | 69.06 | 64.65 | 22.34 | 23.02 | 27.96 | 24.44 |
| 2815314 | 0.00 | 0.00 | 0.00 | 100.00 | 100.00 | 100.00 | 100.00 |
| 2815315 | 11.89 | 15.26 | 14.39 | 86.74 | 82.99 | 83.97 | 84.57 |
| 2815316 | 52.99 | 49.33 | 49.35 | 40.88 | 45.01 | 45.00 | 43.63 |
| 2815317 | 47.02 | 48.03 | 32.86 | 47.53 | 46.46 | 63.38 | 52.46 |
| 2853801 | 6.27 | 4.69 | 4.68 | 93.01 | 94.77 | 94.78 | 94.19 |
| 2853802 | 51.19 | 48.38 | 44.92 | 42.88 | 46.07 | 49.94 | 46.30 |
| 2853803 | 64.62 | 65.20 | 55.25 | 27.90 | 27.33 | 38.43 | 31.22 |
| 2853804 | 12.67 | 17.29 | 12.05 | 85.86 | 80.73 | 86.58 | 84.39 |
| 2853805 | 0.00 | 0.00 | 0.00 | 100.00 | 100.00 | 100.00 | 100.00 |
| 2853806 | 85.71 | 81.25 | 80.00 | 4.36 | 9.44 | 10.85 | 8.22 |
| 2853807 | 14.28 | 16.51 | 11.84 | 84.07 | 81.60 | 86.81 | 84.16 |
| 2853808 | 11.17 | 14.07 | 13.92 | 87.54 | 84.31 | 84.48 | 85.44 |
| 2853809 | 45.15 | 41.73 | 35.40 | 49.62 | 53.48 | 60.55 | 54.55 |
| 2853810 | 11.53 | 10.93 | 14.58 | 87.14 | 87.82 | 83.75 | 86.23 |
| 2853811 | 37.70 | 34.00 | 29.41 | 57.93 | 62.10 | 67.22 | 62.42 |
| 2853812 | 17.07 | 12.28 | 11.76 | 80.95 | 86.31 | 86.89 | 84.72 |
| 2853813 | 20.57 | 22.04 | 29.05 | 77.05 | 75.44 | 67.63 | 73.37 |
| 2853814 | 23.48 | 21.00 | 19.88 | 73.80 | 76.59 | 77.85 | 76.08 |
| 2853815 | 83.65 | 83.25 | 81.91 | 6.67 | 7.21 | 8.72 | 7.53 |
| 2853816 | 27.27 | 28.67 | 31.03 | 69.57 | 68.04 | 65.42 | 67.68 |
| 2853817 | 10.75 | 8.00 | 15.91 | 88.00 | 91.08 | 82.27 | 87.12 |
| 2853818 | 10.34 | 1.85 | 6.06 | 88.46 | 97.94 | 93.25 | 93.21 |
| 2853819 | 1.78 | 9.38 | 6.11 | 98.02 | 89.55 | 93.19 | 93.59 |
| 2853820 | 9.19 | 8.62 | 7.40 | 89.75 | 90.39 | 91.76 | 90.63 |
| 2853821 | 12.96 | 11.09 | 9.08 | 85.54 | 87.64 | 89.89 | 87.69 |
| 2853822 | 16.19 | 16.33 | 17.02 | 81.93 | 81.80 | 81.03 | 81.59 |
| 2853823 | 17.28 | 16.97 | 15.49 | 80.72 | 81.08 | 82.74 | 81.52 |
| 2853824 | 19.85 | 18.39 | 20.72 | 77.86 | 79.50 | 76.91 | 78.09 |
| 2853825 | 0.00 | 0.00 | 0.00 | 100.00 | 100.00 | 100.00 | 100.00 |
| 2853826 | 11.88 | 17.65 | 11.11 | 86.74 | 80.33 | 87.62 | 84.90 |
| 2853827 | 4.09 | 8.00 | 9.45 | 95.44 | 91.08 | 89.47 | 92.00 |
| 2853828 | 9.64 | 7.81 | 8.82 | 89.25 | 91.29 | 90.17 | 90.23 |
| 2853829 | 9.41 | 7.42 | 4.79 | 89.50 | 91.73 | 94.67 | 91.97 |
| 2853830 | 48.71 | 42.02 | 40.27 | 45.65 | 53.16 | 55.12 | 51.31 |
| 2853831 | 18.13 | 14.09 | 9.82 | 79.77 | 84.29 | 89.06 | 84.37 |
| 2853832 | 5.58 | 4.99 | 4.58 | 93.78 | 94.44 | 94.90 | 94.37 |
| 2853833 | 26.67 | 22.22 | 13.04 | 70.25 | 75.23 | 85.46 | 76.98 |
| 2853834 | 7.18 | 7.11 | 7.14 | 91.98 | 92.08 | 92.04 | 92.03 |
| 2853835 | 8.33 | 8.57 | 9.09 | 90.70 | 90.45 | 89.87 | 90.34 |
| 2853836 | 10.00 | 16.00 | 12.24 | 88.84 | 82.17 | 86.35 | 85.79 |
| 2853837 | 5.54 | 5.59 | 10.00 | 93.82 | 93.76 | 88.86 | 92.15 |
| 2853838 | 12.05 | 16.65 | 12.20 | 86.55 | 81.44 | 86.40 | 84.80 |
| 2853839 | 0.00 | 0.00 | 0.00 | 100.00 | 100.00 | 100.00 | 100.00 |
| 2853840 | 16.13 | 16.97 | 14.54 | 82.01 | 81.08 | 83.80 | 82.30 |
| 2853841 | 19.67 | 18.31 | 17.67 | 78.06 | 79.59 | 80.30 | 79.32 |
| 2853842 | 21.37 | 18.95 | 16.64 | 76.16 | 78.88 | 81.46 | 78.83 |
| 2853843 | 11.11 | 16.09 | 8.70 | 87.60 | 82.06 | 90.31 | 86.66 |
| 2853844 | 17.81 | 9.21 | 19.67 | 80.13 | 89.73 | 78.08 | 82.65 |
| 2853845 | 75.56 | 72.02 | 73.91 | 15.69 | 19.72 | 17.64 | 17.69 |
| 2853846 | 34.25 | 32.66 | 33.75 | 61.79 | 63.60 | 62.39 | 62.59 |
| 2853847 | 66.42 | 65.25 | 65.76 | 25.89 | 27.27 | 26.71 | 26.62 |

TABLE 8
Effect of peptide inhibitors on the number of CSCs and cell viability of MDA-MB-231 cells (n = 1)

| | | | COI, Single | | COI, Live Single | | |
|---|---|---|---|---|---|---|---|
| | Total Cell | COI (Cell | COI, Live Single | CD44^{hi}CD24^{lo} CSCs | CD44^{hi}CD24^{lo} CSCs | | |
| Peptide | Count | Count) | Count) | Cells (Cell Count) | (Cell Count) | (%) | Viable Cells (%) |
| Ctrl | 10000 | 8374 | 7324 | 7265 | 6836 | 94.09 | 99.19 |
| P1 | 4339 | 2038 | 1523 | 1395 | 220 | 15.77 | 91.60 |
| P2 | 12706 | 6317 | 4580 | 4402 | 616 | 13.99 | 96.11 |
| P3 | 20311 | 6874 | 5237 | 5099 | 410 | 8.04 | 97.36 |
| P4 | 7923 | 4910 | 3903 | 3777 | 3311 | 87.66 | 96.77 |
| 2815302 | 5074 | 516 | 466 | 438 | 86 | 19.63 | 93.99 |
| 2815303 | 10195 | 18 | 17 | 8 | 2 | 25.00 | 47.06 |
| 2815304 | 4317 | 157 | 137 | 122 | 83 | 68.03 | 89.05 |
| 2815305 | 2870 | 129 | 121 | 88 | 82 | 93.18 | 72.73 |
| 2815306 | 9352 | 323 | 280 | 238 | 66 | 27.73 | 85.00 |
| 2815307 | 10681 | 182 | 164 | 137 | 86 | 62.77 | 83.54 |
| 2815308 | 9921 | 340 | 301 | 163 | 89 | 54.60 | 54.15 |
| 2815309 | 4158 | 74 | 71 | 1 | 1 | 0.00 | 1.41 |
| 2815310 | 8883 | 18 | 16 | 7 | 4 | 57.14 | 43.75 |
| 2815318 | 3480 | 183 | 142 | 132 | 70 | 53.03 | 92.96 |
| 2815312 | 2993 | 9 | 7 | 6 | 0 | 0.00 | 85.71 |
| 2815313 | 8909 | 165 | 148 | 130 | 80 | 61.54 | 87.84 |
| 2815314 | 5611 | 10 | 9 | 2 | 0 | 0.00 | 22.22 |
| 2815315 | 12122 | 51 | 41 | 16 | 3 | 18.75 | 39.02 |
| 2815316 | 10777 | 22 | 20 | 4 | 2 | 50.00 | 20.00 |
| 2815317 | 8821 | 88 | 79 | 44 | 18 | 40.91 | 55.70 |
| 2853801 | 8558 | 2375 | 2024 | 1894 | 102 | 5.39 | 93.58 |
| 2853802 | 9672 | 7612 | 7095 | 6432 | 3133 | 48.71 | 90.66 |
| 2853803 | 1115 | 741 | 702 | 666 | 402 | 60.36 | 94.87 |
| 2853804 | 1384 | 360 | 203 | 138 | 19 | 13.77 | 67.98 |
| 2853805 | 3221 | 52 | 32 | 1 | 0 | 0.00 | 3.13 |
| 2853806 | 4893 | 3997 | 3696 | 3659 | 3361 | 91.86 | 99.00 |
| 2853807 | 3558 | 690 | 439 | 168 | 19 | 11.31 | 38.27 |
| 2853808 | 1192 | 323 | 279 | 242 | 26 | 10.74 | 86.74 |
| 2853809 | 3548 | 1736 | 1560 | 1508 | 537 | 35.61 | 96.67 |
| 2853810 | 3451 | 778 | 613 | 545 | 74 | 13.58 | 88.91 |
| 2853811 | 1274 | 79 | 69 | 64 | 25 | 39.06 | 92.75 |
| 2853812 | 3794 | 108 | 87 | 84 | 14 | 16.67 | 96.55 |
| 2853813 | 867 | 55 | 48 | 18 | 5 | 27.78 | 37.50 |
| 2853814 | 999 | 437 | 387 | 368 | 51 | 13.86 | 95.09 |
| 2853815 | 1114 | 320 | 300 | 279 | 199 | 71.33 | 93.00 |
| 2853816 | 1747 | 160 | 130 | 96 | 12 | 12.50 | 73.85 |
| 2853817 | 899 | 511 | 463 | 407 | 56 | 13.76 | 87.90 |
| 2853818 | 275 | 33 | 25 | 20 | 5 | 25.00 | 80.00 |
| 2853819 | 4572 | 563 | 372 | 191 | 5 | 2.62 | 51.34 |
| 2853820 | 3098 | 541 | 346 | 211 | 14 | 6.64 | 60.98 |
| 2853821 | 7421 | 2756 | 2240 | 2223 | 289 | 13.00 | 99.24 |
| 2853822 | 3204 | 768 | 657 | 648 | 31 | 4.78 | 98.63 |
| 2853823 | 1209 | 672 | 626 | 608 | 22 | 3.62 | 97.12 |
| 2853824 | 5455 | 4232 | 3768 | 3743 | 619 | 16.54 | 99.34 |
| 2853825 | 1583 | 18 | 15 | 0 | 0 | 0.00 | 0.00 |
| 2853826 | 3961 | 554 | 433 | 160 | 5 | 3.13 | 36.95 |
| 2853827 | 4681 | 170 | 110 | 61 | 1 | 1.64 | 55.45 |
| 2853828 | 3309 | 182 | 146 | 28 | 3 | 10.71 | 19.18 |
| 2853829 | 10000 | 167 | 131 | 68 | 4 | 5.88 | 51.91 |
| 2853830 | 8489 | 5072 | 4600 | 4491 | 1312 | 29.21 | 97.63 |
| 2853831 | 10000 | 377 | 280 | 93 | 11 | 11.83 | 33.21 |
| 2853832 | 2068 | 674 | 515 | 444 | 11 | 2.48 | 86.21 |
| 2853833 | 4559 | 495 | 363 | 72 | 9 | 12.50 | 19.83 |
| 2853834 | 4207 | 890 | 660 | 382 | 22 | 5.76 | 57.88 |
| 2853835 | 8580 | 1733 | 1292 | 40 | 1 | 2.50 | 3.10 |
| 2853836 | 5448 | 761 | 547 | 86 | 8 | 9.30 | 15.72 |
| 2853837 | 4805 | 452 | 364 | 235 | 13 | 5.53 | 64.56 |
| 2853838 | 888 | 408 | 333 | 305 | 31 | 10.16 | 91.59 |
| 2853839 | 619 | 12 | 10 | 2 | 0 | 0.00 | 20.00 |
| 2853840 | 3715 | 709 | 487 | 308 | 58 | 18.83 | 63.24 |
| 2853841 | 6480 | 2072 | 1594 | 1289 | 147 | 11.40 | 80.87 |
| 2853842 | 3873 | 325 | 249 | 218 | 27 | 12.39 | 87.55 |
| 2853843 | 9152 | 402 | 298 | 80 | 6 | 7.50 | 26.85 |
| 2853844 | 2094 | 147 | 101 | 66 | 7 | 10.61 | 65.35 |
| 2853845 | 5598 | 4600 | 4111 | 4012 | 2612 | 65.10 | 97.59 |
| 2853846 | 10000 | 370 | 330 | 312 | 87 | 27.88 | 94.55 |
| 2853847 | 10000 | 5345 | 4268 | 4218 | 2640 | 62.59 | 98.83 |

The point mutations were generally well tolerated, with the majority of the peptides retaining the ability to inhibit cancer stem cells (CD44^{hi}CD24^{lo} CSCs) (Table 7). Interestingly, mutation of the lysine acetylation target to alanine (P1₂₋₁₇[K9A]) retained the ability to inhibit cancer stem cells, indicating that the surrounding residues also contribute to the activity of the peptides.

In addition, peptides 2853805 (P1[T2K]), 2853825 (P1[R8L]), 2853839 (P1[V15K]), 2815309 (P1₂₋₁₇[K9A]), 2815312 (P1₂₋₁₇[M12A]) and 2815314 (P1₂₋₁₇[D14A]) killed the majority of the total MDA-MB-231 cells, not only the CSCs (Tables 7 and 8).

To further investigate the cytotoxicity of these peptides, their effect on MCF7 cells, which are epithelial, non-CSC breast cancer cells, was determined (Table 9). Only peptide 2853825 (P1[R8L]) was cytotoxic to MCF7 cells, suggesting that this peptide likely targets both CSC and non-CSC cancer cells. Although not cytotoxic in non-induced MCF7 cells, the remaining peptides inhibited CSC formation in induced MCF7 cells (Table 10).

TABLE 9
Effect of peptide inhibitors on the number of CSCs and cell viability of MCF7 cells

| | | | | COI/Single | COI/Single | | |
|---|---|---|---|---|---|---|---|
| | | | COI/Single Cell\| | Cells/Live\| | Cells/Live/CD44^{hi}CD24^{lo} | CD44^{hi}CD24^{lo} | % Viable |
| Peptide | Total\|Count | COI\|Count | Count | Count | CSCs\|Count | CSC % | Cells |
| Control | 10000 | 6771 | 5505 | 5251 | 370 | 7.05 | 95.39 |
| P1 | 10000 | 6567 | 5585 | 5342 | 233 | 4.36 | 95.65 |
| P2 | 10000 | 5603 | 4482 | 4356 | 123 | 2.82 | 97.19 |
| P3 | 10000 | 6007 | 5039 | 4962 | 162 | 3.26 | 98.47 |
| 2815309 | 10000 | 5425 | 4124 | 4000 | 134 | 3.35 | 96.99 |
| 2815312 | 10000 | 5333 | 4517 | 4331 | 142 | 3.28 | 95.88 |
| 2815314 | 10000 | 6564 | 5443 | 5276 | 145 | 2.75 | 96.93 |
| 2853805 | 10000 | 5356 | 4637 | 4320 | 108 | 2.50 | 93.16 |
| 2853825 | 10000 | 72 | 35 | 0 | 0 | 0.00 | 0.00 |
| 2853839 | 10000 | 3128 | 2294 | 2200 | 25 | 1.14 | 95.90 |

TABLE 10
Effect of peptide inhibitors on the number of CSCs in induced
MCF7 cells

| Peptide | CSC inhibition (%) |
|---|---|
| P1 | 38.10 |
| P2 | 59.93 |
| P3 | 53.67 |
| 2815309 | 52.46 |
| 2815312 | 53.47 |
| 2815314 | 61.00 |
| 2853805 | 64.52 |
| 2853825 | Cytotoxic |
| 2853839 | 83.87 |

### Materials and Methods

All materials and reagents used are readily available from commercial sources such as Sigma-Aldrich, Santa Cruz Biotechnology, Abcam, etc., unless otherwise indicated.

### Cell Culture

MCF7 and MDA-MB-231 cells were obtained from the American Type Culture Collection (Manassas, Va.). Cells were cultured in DMEM (Invitrogen, Life Technologies, Carlsbad, Calif.) supplemented with 10% FBS, 2 mM L-glutamine, and 1% penicillin-streptomycin-neomycin. Stimulated MCF7 cells were generated by treating with 1.32 ng/ml phorbol 12-myristate 13-acetate (PMA) (Sigma-Aldrich, St Louis, Mo.) for 60 h. 4T1 cells were obtained from an in vivo 4T1 metastatic cancer mouse model and cultured in DMEM supplemented with 10% FBS, 2 mM L-glutamine, and 1% penicillin-streptomycin-neomycin. T-47D cells were cultured in DMEM supplemented with 10% FBS, 2 mM L-glutamine, and 1% penicillin-streptomycin-neomycin. Docetaxel resistant cells lines were obtained from a collaborator. Abraxane resistant 4T1 cells (4T1 Group B) were generated by treating with 30 mg/kg abraxane.

Isolation of CTCs from Metastatic Breast Cancer or Melanoma Patient Liquid Biopsies

Melanoma or breast cancer biopsies were pre-enriched using the RosetteSep™ method to isolate CTCs by employing the RosetteSep™ Human CD45 Depletion Kit (15162, Stemcell Technologies) to remove CD45+ cells and red blood cells, using density gradient centrifugation with SepMate™-15 (IVD) density gradient tubes (85420, Stemcell Technologies) and Lymphoprep™ density gradient medium (Catalogue Number 07861, Stemcell Technologies). Enriched cells were then cytospun onto a coverslip pre-treated with poly-L-lysine, were fixed then stored in PBS for staining.

### Immunofluorescence Analysis of PD-L1, H3K27ac, H3K4me3 and H3K9me3 in MDA-MB-231 and Stimulated and Non-Stimulated MCF7 Cells

MDA-MB-231 or stimulated or non-stimulated MCF7 Cells were permeabilised by incubating with 1% Triton X-100 for 20 mins. Cells were probed using a rabbit anti-PD-L1 and visualized with a donkey anti-rabbit secondary antibody conjugated to Alexa Fluor 488, or were probed using a rabbit anti-PDL1 and a mouse-anti H3K27ac, H3K4me3 or H3K9me3 and labelled with either a donkey anti-rabbit secondary antibody conjugated to Alexa Fluor 488 or a donkey anti-mouse secondary antibody conjugated to Alexa Fluor 568. Cover slips were mounted on glass microscope slides with ProLong Diamond Antifade reagent (Life Technologies). Protein targets were localized by confocal laser scanning microscopy. Single 0.5 μm sections were obtained using a Leica DMI8 microscope using 100× oil immersion lens running LAX software. The final image was obtained by averaging four sequential images of the same section. Digital images were analysed using ImageJ software (ImageJ, NIH, Bethesda, Md., USA) to determine either the Total Nuclear Fluorescent Intensity (TNFI) or the Total Cytoplasmic Fluorescent Intensity (TCFI). ImageJ software with automatic thresholding and manual selection of regions of interest (ROIs) specific for cell nuclei was used to calculate the Pearson's co-efficient correlation (PCC) for each pair of antibodies. PCC values range from: −1=inverse of co-localization, 0=no co-localization, +1=perfect co-localization. A minimum of n=20 cells was used for each sample set. The Mann-Whitney nonparametric test (GraphPad Prism, GraphPad Software, San Diego, Calif.) was used to determine significant differences between datasets.

MDA-MB-231 Mouse Xenografts Treated with Abraxane or Docetaxel and Immunofluorescence Analysis thereof

Five-week-old female nude mice were acquired and allowed to acclimatize for one week in the animal facility before experimentation. All experimental procedures were accessed and approved by The Australian National University Animal Experimental Ethics Committee (Ethics ID A2014/30). MDA-MB-231 human breast carcinoma cells were injected subcutaneously into the right mammary gland (2×10⁶ cells in 1:1 PBS and BD Matrigel Matrix). Tumors were measured using external calipers and calculated using the modified ellipsoidal formula: ½ (a/b2), where a=longest diameter and b=shortest diameter. Tumors were allowed to grow to around 50 mm³ before commencing treatments (around 15 days). Abraxane (60 mg/kg) or Docetaxel (10 mg/kg) were administered by i.p. injection. Tumors were excised and collected in DMEM supplemented with 2.5% FCS. Tumors were then finely minced using a surgical blade and incubated at 37° C. for 1 hour in DMEM, 2.5% FCS and collagenase type 4 (Worthington-Biochem) (1 mg of collagenase/1 g of tumor). Digested tumors were spun and resuspended in DMEM, 2.5% FCS before being passed through a 0.2 μM filter and PD-L1 nuclear localization was assessed using immunofluorescence microscopy as described supra.

Immunofluorescence Analysis of PD-L1 and CSV in MICs Isolated from Metastatic Breast Cancer or Melanoma Patient Liquid Biopsies

MICs were fixed with 3.7% formaldehyde and permeabilized with 2% Triton-X-100, then probed with primary mouse antibodies to CSV or primary goat antibodies to PD-L1, followed by the corresponding secondary antibody conjugated to anti-mouse Alexa-Fluor 568 or anti-goat Alexa-Fluor 633. Confocal laser scanning microscopy was used to measure the TNFI, TCFI and Fn/c (nuclear/cytoplasmic fluorescence ratio, calculated using the equation Fn/c=(Fn-Fb)/Fc-Fb) where Fn is the nuclear fluorescence, Fc is the cytoplasmic fluorescence and Fb is the background fluorescence; wherein a value over 1 indicates nuclear bias) as described supra. At least 5-10 individual cells were analyzed per sample.

### Preparation of a Plasmid Containing the Wild-Type PD-L1 Sequence and a Plasmid Containing a PD-L1 [K263Q] Mutant Sequence

PD-L1 sequences (wild-type or PD-L1 [K263Q] mutant) were ligated into the vector pTracer-CMV-BSD and used to transform electro-competent DH10B ElectroMAX cells (Life Technologies; catalogue no. 18290015). Transformed bacteria were used to grow large stocks of plasmid purified/extracted using a Qiagen Plasmid Mega purification kit (Qiagen NV, Hilden, Germany; catalogue no. 12183). MCF7 cells were transfected with a plasmid containing the wild-type PD-L1 sequence, a plasmid containing a PD-L1 [K263Q] mutant (Mut1) or vector only (VO) employing the NEON Plasmid electroporation transfection system (Life Technologies; catalogue no. MPK5000).

Immunofluorescence Analysis of PD-L1 in MCF7 Cells Transfected with Plasmids Containing the Wild-Type PD-L1 Sequence, and a PD-L1 [K263Q] Mutant Sequence

MCF7 cells were transfected with a plasmid containing the wild-type PD-L1 sequence, a plasmid containing a PD-L1 [K263Q] mutant (Mut1) or vector only (VO) using the NEON electroporation transfection system (Life Technologies). Transfected cells were treated with vehicle only (non-stimulated) or stimulated with 1.32 ng/mL phorbol 12-myristate 13-acetate (PMA) (Sigma-Aldrich, St Louis, Mo.) for 60 h. Cells were fixed with 3.7% formaldehyde and permeabilized with 2% Triton-X-100 for 20 mins, then probed with primary mouse antibodies to CSV or CD133, primary goat antibodies to SNAI1, or primary rabbit antibodies to PD-L1 or EGFR, followed by the corresponding secondary antibody conjugated to anti-mouse Alexa-Fluor 568, anti-goat Alexa-Fluor 633 or anti-rabbit Alexa-Fluor 488. Confocal laser scanning microscopy was used to measure the TNFI, TCFI and Fn/c as described supra. At least 5-10 individual cells were analyzed per sample.

### WST-1 Cell Proliferation Assay

MCF7 cells were transfected with a plasmid containing the wild-type PD-L1 sequence, a plasmid containing a PD-L1 [K263Q] mutant (Mut1) or vector only (VO) using the NEON electroporation transfection system (Life Technologies). Transfected cells were used for the colorimetric based WTS-1 proliferation assay (Sigma-Aldrich) to examine the effect of transfection on MCF7 cell proliferation. The WTS-1 proliferation assay was also used to determine the effect of treatment with P1, P2 and P3 (synthesized in accordance with Example 5) on MDA-MB-231 cell proliferation. The stable tetrazolium salt WST-1 is cleaved to a soluble formazan by a complex cellular mechanism that occurs primarily at the cell surface. This bioreduction is largely dependent on the glycolytic production of NAD(P)H in viable cells. Therefore, the amount of formazan dye formed directly correlates to the number of metabolically active cells in the culture. Cells grown in a 96-well tissue culture plate were treated with vehicle only or 6.25, 12.5, 25, 50, 100, 200 or 400 μM of P1, P2 or P3 for 72 hours at 37° C. in a humidified CO₂ incubator. Cells were then incubated with the WST-1 reagent for 0.5-4 hours. Following incubation, the formazan dye formed is quantitated with a scanning multi-well spectrophotometer (ELISA reader). The measured absorbance directly correlates to the number of viable cells.

### Generation of Antibodies Specific for PD-L1 Trimethylated at Lysine 263 and PD-L1 Acetylated at Lysine 263

Antibodies were generated against peptides 2803201, 2803204 and 2803213 (Table 11). As short peptides are generally not immunogenic in their own right it is necessary to couple them to immunogenic carrier proteins. To facilitate this coupling, a cysteine was incorporated at the C-terminus of the peptide and reacted to conjugate the peptide to an immunogenic carrier protein, Keyhole Limpet Hemocyanin (KLH). No special immunization protocols were required to generate anti-trimethylated or anti-acetylated peptide antibodies. Two rabbits for each peptide sequence were immunized several weeks apart. The first immunization is with an emulsion of the peptide conjugate with Complete Freunds adjuvant, the second using Incomplete Freunds adjuvant. Potent anti-peptide sera are obtained after several weeks (refer to Palfreyman, et al. (1984) J Immunol Meth, 75:383).

TABLE 11
Peptide sequences used for antibody generation

| | | Molecular Weight |
|---|---|---|
| Peptide | Sequence | (Da) |
| 2803201 | FRLRKGRMMDVKKC-OH | 1768.25 |
| 2803204 | FRLRK(Ac)GRMMDVKKC-OH | 1810.29 |
| 2803213 | FRLRK(Me3)GRMMDVKKC-OH | 1811.34 |

The testing of trimethylated and acetylated peptide antisera is performed using an enzyme linked immunosorbent assay (ELISA) where the sera are titrated on microtiter plates coated with non-trimethylated peptide and trimethylated peptide, or non-acetylated and acetylated peptide.

Antibody enhancement is performed by coupling the non-trimethylated, non-acetylated analogue of the peptide used for the immunization to a gel Sulfo Link Coupling Resin (Thermo Scientific, Catalogue number 20401) using the available cysteine residue, following the manufacturers instructions. The resultant gel is incubated with aliquots of the antisera to absorb antibodies specific to the non-trimethylated, non-acetylated peptide. The resultant antiserum will have an enhanced specificity for the trimethylated peptide or acetylated peptide sequence.

To produce affinity purified antibodies that are specific to the trimethylated or acetylated peptide only, it is necessary to first perform the enhancement procedure to remove antibodies from the serum that are specific to the non-tri methylated and non-acetylated peptide. Specificity of the affinity purified antibodies are tested by ELISA back onto both the non-trimethylated and the trimethylated peptides, or non-acetylated and acetylated peptides coated onto the plate. Generated antibodies showed high specificity for trimethylated PD-L1 and acetylated PD-L1 at residue 263.

Immunofluorescence Analysis of PD-L1 Acetylated at Lysine 263 (Acetylated PD-L1) and PD-L1 Trimethylated at Lysine 263 (Trimethylated PD-L1) in MDA-MB-231 cells, Non-Permeabilized MDA-MB-231 Cells, CTCs Isolated From Metastatic Breast Cancer Patients and CTCs Isolated From Melanoma Patients

Permeabilized MDA-MB-231 cells were generated by incubating with 1% Triton X-100 for 20 min. Melanoma patient samples were classified into responder, primary resistance and secondary resistance cohorts based on RECIST 1.1 CT Scan measurements of the size of the solid tumor measured at multiple points after treatment. Responder means the tumor is shrinking, primary resistance means the tumor does not shrink and is increasing in size, whereas secondary resistance has a responder response at first followed by a resistance and tumor growth. Melanoma and metastatic breast cancer CTCs were obtained from patient liquid biopsies isolated using CD45-depletion Rosette Lymphopep (STEMCELL) cell isolation kits. Cells were probed with rabbit anti-PD-L1 (Santa Cruz Biotechnology), rabbit anti-acetylated PD-L1 or rabbit anti-trimethylated PD-L1 (generated as described above) and visualized with a donkey anti-rabbit AF 488. Cover slips were mounted on glass microscope slides with ProLong Diamond Antifade reagent (Life Technologies). Protein targets were localized by confocal laser scanning microscopy. Single 0.5 pm sections were obtained using a Leica DMI8 microscope using 100x oil immersion lens running LAX software. The final image was obtained by averaging four sequential images of the same section. Digital images were analyzed using ImageJ software as described above.

Immunofluorescence Analysis of PD-L1 and Acetylated PD-L1 in MDA-MB-231 Cells Treated with P1, P2, P3 and P4

1×10⁴ MDA-MB-231 cells were seeded onto coverslips in 12 well plates with DMEM media overnight. Cells were treated with 50 μM P1, P2, P3 or P4 or vehicle (water) for 72 hours. Treated cells were fixed with 3.7% formaldehyde, and then permeabilised by incubating with 1% Triton X-100 for 20 min. Cells were then probed with a rabbit anti-PD-L1 antibody (Santa Cruz Biotechnology) or rabbit anti-acetylated PD-L1 (generated as described above) and visualized with a donkey anti-rabbit AF 488. Cover slips were mounted on glass microscope slides with ProLong Diamond Antifade reagent (Life Technologies). Protein targets were localized using confocal laser scanning microscopy. Single 0.5 μm sections were obtained using a Leica DMI8 microscope using 100× oil immersion lens running LAX software. The final image was obtained by averaging four sequential images of the same section. Digital images were analyzed using ImageJ software as described above.

### FACS Analysis of the Cancer Stem Cell Phenotype (CD44high/CD24low) in MDA-MB-231 Cells in Response to P1, P2 and P3 Treatment

5×10⁴ MDA-MB-231 cells were seeded with 1 mL of complete DMEM in 12 well plates overnight. MDA-MB-231 cells were treated with 6.25, 12.5, 25, 50, 100, 200 or 400 μM P1, P2 or P3 (synthesized in accordance with Example 5) or vehicle only for 72 hours. Samples were harvested by trypsinization followed by washing with DPBS containing 2% HI-FBS. FACS staining was performed using anti-human CD44-APC, anti-human CD24-PE, Hoechst and anti-human EpCAM antibody cocktails. Data was collected from a BD-FACSLSR-II flow cytometer. Treestar FlowJo was used for data analysis.

FACS Analysis of the Cancer Stem Cell Phenotype and Cell Viability in MDA-MB-231 Cells and MCF7 Cells in Response to Treatment with P1 Analogues

P1 Analogues were synthesized in accordance with the procedure of Example 5. 4×10⁴ MDA-MB-231 cells or 1×10⁴ induced or non-induced MCF7 cells were seeded in a 12 well plate with 1 mL complete media overnight. Induced MCF7 cells were prepared by stimulating cells with PMA/TGF-β and incubating for 12 hours. Cells were treated with 50 μM test peptide (dissolved in water) for 48 hours. Cells were then washed twice with 1 mL PBS and harvested by trypsinisation. Non-adherent cells were removed during the washing steps. Adherent cells were collected, and were subsequently stained with anti-human CD44, CD24 and Hoechst 33342 antibodies on ice for 30 mins. FACS was performed on BD FACS LSRII to measure the percentage of CD44^{hi}CD24^{lo} CSCs by dividing CD44^{hi}CD24^{lo} events by total live cell counts (Hoechst negative population) (as described in Fillmore and Kuperwasser (2007) Breast Cancer Res, 9: 303). The percentage of viable cells was calculated by dividing total live cell events by total single cells as described previously (Siemann and Keng (1986), Cancer Res, 46: 3556-3559).

Immunofluorescence Analysis of CSV, PD-L1, EGFR, SNAI1, EHTM2, DMNTI, SETDB1, H3K9me3, 5-methylcytosine and ABCB5 in MDA-MB-231 Cells in Response to P1, P2, P3 and P4 Treatment

1×10⁴ MDA-MB-231 cells were seeded onto coverslips in 12 well plates with DMEM media. Cells were treated with 50 μM P1, P2, P3 or P4 (synthesized in accordance with Example 5) or vehicle (water) for 72 hours. Cells were fixed with 3.7% formaldehyde and permeabilized with 2% Triton-X-100, then probed with primary mouse antibodies to CSV, DMNT1, H3K9me3 or 5-methylcytosine; primary goat antibodies to SNAI1, SETDB1 or ABCB5; or primary rabbit antibodies to PD-L1, EGFR, EHMT2; followed by the corresponding secondary antibody conjugated to anti-mouse Alexa-Fluor 568, anti-goat Alexa-Fluor 633 or anti-rabbit Alexa-Fluor 488. Confocal laser scanning microscopy was used to measure the TFI, TNFI and TCFI as described above. At least 20 individual cells were analyzed per sample.

Immunofluorescence Analysis of p300 and Acetylated PD-L1 in CTCs from Metastatic Melanoma Patients and Metastatic Breast Cancer Cells

CTCs were isolated from metastatic melanoma biopsies as described above. Melanoma CTCs isolated from liquid biopsies were divided into three cohorts based on response to immunotherapy. Responders (responds to immunotherapy), resistant (no response to immunotherapy, cancer is refractive or initial response followed by refractory disease and cancer no longer responds). Melanoma CTCs were permeabilized by incubating with 1% Triton X-100 for 20 mins and were probed with rabbit anti-acetylated PD-L1 (prepared as described above) or mouse anti-p300 and visualized with a donkey anti-rabbit Alexa-Fluor 488 or anti-mouse Alexa-Fluor 568. Matched naïve (MCF7, MDA-MB-231, T-47D and 4T1 group A), docetaxel resistant (MCF7 TXT50, MDA-MB-231 TXT50 and T-47D TXT50), and abraxane resistant (4T1 group B) metastatic breast cancer cells were permeabilized by incubating with 1% Triton X-100 for 20 mins and were probed with rabbit anti-acetylated PD-L1 (prepared as described above) or mouse anti-p300 and visualized with a donkey anti-rabbit Alexa-Fluor 488 or anti-mouse Alexa-Fluor 568. Cover slips were mounted on glass microscope slides with ProLong Diamond Antifade reagent (Life Technologies). Protein targets were localized by confocal laser scanning microscopy as described above. ImageJ software was used to determine the PCC(r), TNFI, TCFI or TFI as described above.

Immunofluorescence Analysis of p300 localization in Response to P1, P2, P3 and P4 Treatment

MDA-MB-231 cells were treated with 50 μm of P1, P2, P3, P4 or vehicle. Cells were fixed with 3.7% formaldehyde and permeabilized with 1% Triton-X-100 for 20 mins and were probed with rabbit anti-acetylated PD-L1 (prepared as described above), mouse anti-p300 and visualized with a donkey anti-rabbit Alexa-Fluor 488 or anti-mouse Alexa-Fluor 568. Cover slips were mounted on glass microscope slides with ProLong Diamond Antifade reagent (Life Technologies). Protein targets were localized by confocal laser scanning microscopy as described above. TNFI and PCC(r) were calculated using ImageJ software as previously described.

### Immunofluorescence Analysis of PD-1 in Jurkat T-Cells or OT1 Derived T-Cells

Naïve Jurkat T Cells, Jurkat T cells stimulated with PMA/I, naïve OT1 derived T-cells, or OT1 virus treated influenza specific effector cells were fixed with 3.7% formaldehyde and permeabilized with 2% Triton-X-100, then probed with a primary mouse antibody to PD-1 directly conjugated to Alexa-Fluor 647. Confocal laser scanning microscopy was used to measure the TNFI. At least 20 individual cells were analyzed per sample.

### Immunofluorescence Analysis of PD-1 in Jurkat T-Cells in Response to P1, P2 and P3 Treatment

1×10⁴ Jurkat T-cells were seeded onto coverslips in 12 well plates with DMEM media. Cells were treated with 50 μM P1, P2, P3 (synthesized in accordance with Example 5) or vehicle (water) for 72 hours. Cells were fixed with 3.7% formaldehyde and permeabilized with 2% Triton-X-100, then probed with a primary mouse antibody to PD-1 directly conjugated to Alexa-Fluor 647. Confocal laser scanning microscopy was used to measure the TNFI, TCFI and Fn/c as described above. At least 20 individual cells were analyzed per sample.

### Immunofluorescence Analysis of PD-L2, PD-L1 and CSV in MDA-MB-231 Cells in Response to P1, P2 and P3 Treatment

1×10⁴ MDA-MB-231 cells were treated with 50 μM P1, P2, P3 (synthesized in accordance with Example 5) or vehicle (water) for 72 hours. Cells were fixed with 3.7% formaldehyde and permeabilized with 2% Triton-X-100, then probed with a primary rabbit antibody to PD-L2, primary goat antibody to PD-L1 and primary mouse antibody to CSV, followed by the corresponding secondary antibody conjugated to anti-mouse Alexa-Fluor 568, anti-goat Alexa-Fluor 633 or anti-rabbit Alexa-Fluor 488. Confocal laser scanning microscopy was used to measure the TNFI and TCFI as described above. At least 20 individual cells were analyzed per sample.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A method of inhibiting or reducing nuclear localization of a nuclear localizable polypeptide, wherein acetylation of an acetylation site of the nuclear localizable polypeptide increases its nuclear localization in a cell, comprising contacting the cell with a proteinaceous molecule comprising, consisting or consisting essentially of an amino acid sequence corresponding to an acetylation site.

2. The method according to claim 1, wherein the polypeptide is PD-1.

3. The method according to claim 1, wherein the polypeptide is PD-L1.

4. The method according to claim 1, wherein the polypeptide is PD-L2.

5. A method of inhibiting or reducing the nuclear localization of PD-1, PD-L1 or PD-L2 in a PD-1-, PD-L1- or PD-L2-overexpressing cell, comprising contacting the cell with a proteinaceous molecule comprising, consisting or consisting essentially of an amino acid sequence corresponding to an acetylation site.

6. The method according to claim 5, wherein the PD-1-, PD-L1- or PD-L2-overexpressing cell is a cancer stem cell or a non-cancer stem cell tumor cell.

7. The method according to claim 6, wherein the PD-1-, PD-L1- or PD-L2-overexpressing cell is a cancer stem cell tumor cell.

8. A method of altering at least one of (i) formation; (ii) proliferation; (iii) maintenance; (iv) epithelial to mesenchymal cell transition (EMT); (v) mesenchymal to epithelial cell transition (MET); or (vi) viability of a PD-1-, PD-L1- or PD-L2-overexpressing cell, comprising contacting said cell with a formation-, proliferation-, maintenance-, EMT-, MET-, or viability-modulating amount of a proteinaceous molecule comprising, consisting or consisting essentially of an amino acid sequence corresponding to an acetylation site.

9. The method according to claim 8, wherein EMT is inhibited or reduced.

10. The method according to claim 8 or claim 9, wherein the PD-1-, PD-L1- or PD-L2-overexpressing cell is a cancer stem cell or a non-cancer stem cell tumor cell.

11. The method according to claim 10, wherein the PD-1-, PD-L1- or PD-L2-overexpressing cell is a cancer stem cell tumor cell.

12. The method according to claim 11, wherein formation of a cancer stem cell tumor cell is inhibited or reduced.

13. The method according to claim 11, wherein proliferation of a cancer stem cell tumor cell is inhibited or reduced.

14. The method according to claim 11, wherein the viability of a cancer stem cell tumor cell is inhibited or reduced.

15. A method of treating or preventing a cancer in a subject wherein the cancer comprises at least one PD-1-, PD-L1- or PD-L2-overexpressing cell, comprising administering to the subject a proteinaceous molecule comprising, consisting or consisting essentially of an amino acid sequence corresponding to an acetylation site.

16. The method according to claim 15, wherein the PD-1-, PD-L1- or PD-L2-overexpressing cell is a cancer stem cell or a non-cancer stem cell tumor cell.

17. The method according to claim 16, wherein the PD-1-, PD-L1- or PD-L2-overexpressing cell is a cancer stem cell tumor cell.

18. The method according to any one of claims 15-17, wherein the cancer is selected from breast, prostate, lung, bladder, pancreatic, colon, liver or brain cancer, or melanoma or retinoblastoma.

19. The method according to any one of claims 15-18, further comprising administering one or more further cancer therapies.

20. The method according to claim 19, wherein the further cancer therapy is a chemotherapeutic agent.

21. The method according to any one of claims 1-20, wherein the proteinaceous molecule comprises, consists or consists essentially of an amino acid sequence corresponding to residues 255 to 271 of PD-L1.

22. A method of producing a proteinaceous molecule that inhibits or reduces nuclear localization of a nuclear localizable polypeptide wherein acetylation of an acetylation site of the nuclear localizable polypeptide increases its nuclear localization in a cell, the method comprising:
a) contacting a cell with a proteinaceous molecule comprising, consisting or consisting essentially of an amino acid sequence corresponding to an acetylation site; and
b) detecting a reduction in or inhibition of the nuclear localization of the nuclear localizable polypeptide in the cell relative to a normal or reference level of nuclear localization in the absence of the proteinaceous molecule.

23. The method according to claim 22, wherein the proteinaceous molecule is a fragment of a nuclear localizable polypeptide.

24. The method according to claim 22 or claim 23, wherein the proteinaceous molecule comprises 50 amino acid residues or less.

25. The method according to any one of claims 22-24, wherein the amino acid sequence corresponding to an acetylation site is an amino acid sequence corresponding to residues 255 to 271 of PD-L1.

26. The method according to claim 25, wherein the proteinaceous molecule is distinguished from PD-L1 by the addition, deletion and/or substitution of at least one (e.g. 1, 2, 3, 4, 5, 6, 7, 8, 9 etc.) amino acid in residues 255 to 271 of PD-L1.

27. A method of producing a proteinaceous molecule that inhibits or reduces nuclear localization of a nuclear localizable polypeptide wherein acetylation of an acetylation site of the nuclear localizable polypeptide increases its nuclear localization in a cell, the method comprising:
a) contacting a cell with a proteinaceous molecule comprising, consisting or consisting essentially of an amino acid sequence corresponding to residues 255 to 271 of PD-L1; and
b) detecting a reduction in or inhibition of the nuclear localization of the nuclear localizable polypeptide in the cell relative to a normal or reference level of nuclear localization in the absence of the proteinaceous molecule.

28. The method according to claim 27, wherein the proteinaceous molecule is distinguished from PD-L1 by the addition, deletion and/or substitution of at least one (e.g. 1, 2, 3, 4, 5, 6, 7, 8, 9 etc.) amino acid in residues 255 to 271 of PD-L1.

29. A method of producing a proteinaceous molecule that inhibits or reduces at least one of formation, proliferation, viability or EMT of a cancer stem cell, the method comprising:
a) contacting a cancer stem cell with a proteinaceous molecule comprising, consisting or consisting essentially of an amino acid sequence corresponding to residues 255 to 271 of PD-L1; and
b) detecting a reduction in or inhibition of the formation, proliferation or EMT of the cancer stem cell relative to a normal or reference level of formation, proliferation, viability or EMT of the cell in the absence of the proteinaceous molecule.

30. The method according to claim 29, wherein the proteinaceous molecule is distinguished from PD-L1 by the addition, deletion and/or substitution of at least one (e.g. 1, 2, 3, 4, 5, 6, 7, 8, 9 etc.) amino acid in residues 255 to 271 of PD-L1.

31. An isolated or purified proteinaceous molecule represented by Formula I:
Z₁X₁X₂X₃X₄FX₅X₆X₇X₈X₉X₁₀X₁₁X₁₂X₁₃X₁₄X₁₅X₁₆Z₂ (I)
wherein:
Z₁ and Z₂ are independently absent or are independently selected from at least one of a proteinaceous moiety comprising from about 1 to about 50 amino acid residues (and all integer residues in between), and a protecting moiety;
X₁ is absent or is selected from small amino acid residues including A, G, S, T and modified forms thereof, and hydrophobic amino acid residues including M, Nle, I, L, V, F, Y, W and modified forms thereof;
X₂ is selected from small amino acid residues including A, G, S, T and modified forms thereof, and charged amino acid residues including K, R, D, E and modified forms thereof;
X₃ is selected from any amino acid residue;
X₄ is selected from charged amino acid residues including K, R, D, E and modified forms thereof, and hydrophobic amino acid residues including M, Nle, I, L, V, F, Y, W and modified forms thereof;
X₅ is selected from any amino acid residue;
X₆ is selected from charged amino acid residues including K, R, D, E and modified forms thereof, and hydrophobic amino acid residues including M, Nle, I, L, V, F, Y, W and modified forms thereof;
X₇ is selected from charged amino acid residues including K, R, D, E and modified forms thereof, and hydrophobic amino acid residues including M, Nle, I, L, V, F, Y, W and modified forms thereof;
X₈ is selected from small amino acid residues including A, G, S, T and modified forms thereof, basic amino acid residues including K, R, Orn and modified forms thereof, and amino acid residues with an amide-containing side chain including N, Q, Orn(Ac), K(Ac) and modified forms thereof;
X₉ is selected from small amino acid residues including G, S, T and modified forms thereof, charged amino acid residues including K, R, D, E and modified forms thereof, and hydrophobic amino acid residues including M, Nle, I, L, V, F, Y, W and modified forms thereof;
X₁₀ is selected from any amino acid residue;
X₁₁ is selected from any amino acid residue;
X₁₂ is selected from charged amino acid residues including K, R, D, E and modified forms thereof, and hydrophobic amino acid residues including M, Nle, I, L, V, F, Y, W and modified forms thereof;
X₁₃ is selected from any amino acid residue;
X₁₄ is selected from any amino acid residue;
X₁₅ is selected from charged amino acid residues including K, R, D, E and modified forms thereof, and hydrophobic amino acid residues including M, Nle, I, L, V, F, Y, W and modified forms thereof; and
X₁₆ is selected from basic amino acid residues including K, R and modified forms thereof.

32. The proteinaceous molecule according to claim 31, wherein Z₁ is absent.

33. The proteinaceous molecule according to claim 31 or 32, wherein Z₂ is absent.

34. The proteinaceous molecule according to any one of claims 31-33, wherein X₁ is selected from L and A.

35. The proteinaceous molecule according to any one of claims 31-34, wherein X₁ is absent.

36. The proteinaceous molecule according to any one of claims 31-35, wherein X₂ is selected from small amino acid residues including A, G, S, T and modified forms thereof, and basic amino acid residues including K, R and modified forms thereof.

37. The proteinaceous molecule according to claim 36, wherein X₂ is selected from A and K.

38. The proteinaceous molecule according to claim 37, wherein X₂ is K.

39. The proteinaceous molecule according to any one of claims 31-38, wherein X₃ is selected from charged amino acid residues including K, R, D, E and modified forms thereof, and aromatic amino acid residues including F, Y, W and modified forms thereof.

40. The proteinaceous molecule according to claim 39, wherein X₃ is selected from F, E and K.

41. The proteinaceous molecule according to claim 40, wherein X₃ is F.

42. The proteinaceous molecule according to any one of claims 31-41, wherein X₄ is selected from acidic amino acid residues including D, E and modified forms thereof, and hydrophobic amino acid residues including I, L, V, M, Nle and modified forms thereof.

43. The proteinaceous molecule according to claim 42, wherein X₄ is selected from I, Land E.

44. The proteinaceous molecule according to claim 43, wherein X₄ is I.

45. The proteinaceous molecule according to any one of claims 31-44, wherein X₅ is selected from charged amino acid residues including K, R, D, E and modified forms thereof, and hydrophobic amino acid residues including M, Nle, I, L, V, F, Y, W and modified forms thereof.

46. The proteinaceous molecule according to claim 45, wherein X₅ is selected from R, E and V.

47. The proteinaceous molecule according to claim 46, wherein X₅ is V.

48. The proteinaceous molecule according to any one of claims 31-47, wherein X₆ is selected from L, E, K, F and V.

49. The proteinaceous molecule according to claim 48, wherein X₆ is L or F.

50. The proteinaceous molecule according to claim 49, wherein X₆ is F.

51. The proteinaceous molecule according to any one of claims 31-50, wherein X₇ is selected from R, E and L.

52. The proteinaceous molecule according to claim 51, wherein X₇ is L.

53. The proteinaceous molecule according to any one of claims 31-52, wherein X₈ is selected from small amino acid residues including A, G, S, T and modified forms thereof, and basic amino acid residues including K, R, Orn and modified forms thereof.

54. The proteinaceous molecule according to claim 53, wherein X₈ is A or K.

55. The proteinaceous molecule according to claim 54, wherein X₈ is A.

56. The proteinaceous molecule according to any one of claims 31-55, wherein X₉ is selected from G, acidic amino acid residues including D, E and modified forms thereof, and hydrophobic amino acid residues including M, Nle, I, L, V and modified forms thereof.

57. The proteinaceous molecule according to claim 56, wherein X₉ is G, D or V.

58. The proteinaceous molecule according to any one of claims 31-57, wherein X₁₀ is selected from basic amino acid residues including K, R and modified forms thereof, and hydrophobic amino acid residues including M, Nle, I, L, V, F, Y, W and modified forms thereof.

59. The proteinaceous molecule according to claim 58, wherein X₁₀ is selected from R and V.

60. The proteinaceous molecule according to claim 59, wherein X₁₀ is R.

61. The proteinaceous molecule according to any one of claims 31-60, wherein X₁₁ is selected from small amino acid residues including A, G, S, T and modified forms thereof, hydrophobic amino acid residues including M, Nle, I, L, V, F, Y, W and modified forms thereof, and acidic amino acid residues including D, E and modified forms thereof.

62. The proteinaceous molecule according to claim 61, wherein X₁₁ is selected from M, Nle, A and E.

63. The proteinaceous molecule according to claim 62, wherein X₁₁ is A.

64. The proteinaceous molecule according to any one of claims 31-63, wherein X₁₂ is selected from hydrophobic amino acid residues including M, Nle, I, L, V, F, Y, W and modified forms thereof, and acidic amino acid residues including D, E and modified forms thereof.

65. The proteinaceous molecule according to claim 64, wherein X₁₂ is M, Nle or E.

66. The proteinaceous molecule according to claim 65, wherein X₁₂ is E.

67. The proteinaceous molecule according to any one of claims 31-66, wherein X₁₃ is selected from small amino acid residues including A, G, S, T and modified forms thereof, hydrophobic amino acid residues including M, Nle, I, L, V, F, Y, W and modified forms thereof, and acidic amino acid residues including D, E and modified forms thereof.

68. The proteinaceous molecule according to claim 67, wherein X₁₃ is A, D or V.

69. The proteinaceous molecule according to claim 68, wherein X₁₃ is A.

70. The proteinaceous molecule according to any one of claims 31-69, wherein X₁₄ is selected from hydrophobic amino acid residues including M, Nle, I, L, V, F, Y, W and modified forms thereof, and basic amino acid residues including K, R and modified forms thereof.

71. The proteinaceous molecule according to claim 70, wherein X₁₄ is V or K.

72. The proteinaceous molecule according to claim 71, wherein X₁₄ is K.

73. The proteinaceous molecule according to any one of claims 31-72, wherein X₁₅ is selected from basic amino acid residues including K, R and modified forms thereof, and aromatic amino acid residues including F, Y, W and modified forms thereof.

74. The proteinaceous molecule according to claim 73, wherein X₁₅ is R, K or Y.

75. The proteinaceous molecule according to claim 74, wherein X₁₅ is K.

76. The proteinaceous molecule according to any one of claims 31-75, wherein X₁₆ is K.

77. The proteinaceous molecule according to claim 31, wherein the proteinaceous molecule of Formula I comprises, consists or consists essentially of an amino acid sequence represented by any one of SEQ ID NO: 1-18:
[SEQ ID NO: 1]
LTFIFRLRKGRMMDVKK;
[SEQ ID NO: 2]
LTFIFRLRQGRMMDVKK;
[SEQ ID NO: 3]
LTFIFRLRK(Ac)GRMMDVKK;
[SEQ ID NO: 4]
ATFIFRLRKGRMMDVKK;
[SEQ ID NO: 5]
LKFIFRLRKGRMMDVKK;
[SEQ ID NO: 6]
AFIFRLRKGRMMDVKK;
[SEQ ID NO: 7]
LTFIFVLRKGRMMDVKK;
[SEQ ID NO: 8]
LTFIFRFRKGRMMDVKK;
[SEQ ID NO: 9]
LTFIFRLLKGRMMDVKK;
[SEQ ID NO: 10]
TFIFRLRAGRMMDVKK;
[SEQ ID NO: 11]
LTFIFRLRKDRMMDVKK;
[SEQ ID NO: 12]
LTFIFRLRKVRMMDVKK;
[SEQ ID NO: 13]
TFIFRLRKGRAMDVKK;
[SEQ ID NO: 14]
LTFIFRLRKGREMDVKK;
[SEQ ID NO: 15]
LTFIFRLRKGRM EDVKK;
[SEQ ID NO: 16]
TFIFRLRKGRMMAVKK;
[SEQ ID NO: 17]
LTFIFRLRKGRMMVVKK;
or
[SEQ ID NO: 18]
LTFIFRLRKGRMMDKKK.

78. The proteinaceous molecule according to claim 77, wherein the proteinaceous molecule of Formula I comprises, consists or consists essentially of an amino acid sequence represented by SEQ ID NO: 1, 4, 9, 10, 13, 16 or 18.

79. The proteinaceous molecule according to any one of claims 31-78, wherein the proteinaceous molecule has any one or more activities selected from the group consisting of:
(i) increasing cell death; (ii) increasing MET; (iii) reducing or inhibiting EMT; (iv) inhibiting or reducing maintenance; (v) inhibiting or reducing proliferation; (vi) increasing differentiation;
(vii) inhibiting or reducing formation; or (viii) reducing viability of a PD-1-, PD-L1- or PD-L2-overexpressing cell.

80. The proteinaceous molecule according to claim 79, wherein the cell is a PD-L1-overexpressing cell.

81. The proteinaceous molecule according to claim 79 or claim 80, wherein the cell is a cancer stem cell or a non-cancer stem cell tumor cell.

82. The proteinaceous molecule according to claim 81, wherein the cell is a cancer stem cell tumor cell.

83. The proteinaceous molecule according to any one of claims 31-82, wherein the proteinaceous molecule of Formula I further comprises at least one membrane permeating moiety.

84. The proteinaceous molecule according to claim 83, wherein the membrane permeating moiety is a lipid moiety.

85. The proteinaceous molecule according to claim 84, wherein the membrane permeating moiety is a myristoyl group.

86. The proteinaceous molecule according to any one of claims 83-85, wherein the membrane permeating moiety is coupled to the N- or C-terminal amino acid residue.

87. The proteinaceous molecule according to claim 86, wherein the membrane permeating moiety is coupled to the N-terminal amino acid residue.

88. The method according to claim 21, wherein the proteinaceous molecule is the isolated or purified proteinaceous molecule according to any one of claims 31-87.
